(19) United States
(12) Patent Application Publication
(10) Pub. No.: US 2024/0209093 A1
(43) Pub. Date: Jun. 27, 2024

(54) SINGLE DOMAIN PD-L1 ANTIBODIES

(71) Applicant: I-MAB BIOPHARMA CO., LTD., Shanghai (CN)

(72) Inventors: Wenqing JIANG, Shanghai (CN); Yan LIU, Shanghai (CN); Haijuan GU, Shanghai (CN); Feifei CUI, Shanghai (CN); Zhengyi WANG, Shanghai (CN); Bingshi GUO, Shanghai (CN)

(21) Appl. No.: 18/288,375

(22) PCT Filed: Apr. 26, 2022
(86) PCT No.: PCT/CN2022/089388

§ 371 Date: Oct. 25, 2023

(30) Foreign Application Priority Data
Apr. 26, 2021 (WO) PCT/CN2021/090046
Apr. 26, 2021 (WO) PCT/CN2021/090049
Apr. 26, 2021 (WO) PCT/CN2021/090058

(51) Int. Cl. C07K 16/28; A61K 39/00; A61P 35/00

(52) U.S. Cl. CPC C07K 16/2827; A61P 35/00; A61K 2039/505; C07K 2317/24; C07K 2317/31; C07K 2317/565; C07K 2317/622

(57) ABSTRACT

Provided are single domain anti-PD-L1 antibodies and polypeptides, such as bispecific antibodies and chimeric antigen receptors, that include these single domain antibodies. These antibodies, including their humanized counterparts, exhibited superior activities and are suitable for use in various bispecific antibody formats. Methods of using the antibodies or polypeptides for treating and diagnosing diseases such as cancer and infectious diseases are also provided.

The present invention claims the priority of the PCT/CN2021/090058, filed on Apr. 26, 2021, the contents of which are incorporated herein by its entirety.

The present invention claims the priority of the PCT/CN2021/090046, filed on Apr. 26, 2021, the contents of which are incorporated herein by its entirety.

The present invention claims the priority of the PCT/CN2021/090049, filed on Apr. 26, 2021, the contents of which are incorporated herein by its entirety.

### BACKGROUND

A single domain antibody (sdAb), also known as a nanobody, is an antibody fragment consisting of a single monomeric variable antibody domain. Like a whole antibody, it is able to bind selectively to a specific antigen. With a molecular weight of only 12-15 kDa, single domain antibodies are much smaller than common antibodies (150-160 kDa). Single domain antibodies, given their small sizes and one-chain nature, can be particularly suitable for inclusion as a fragment in other proteins, such as bispecific antibodies.

Antibodies specific to programmed death-ligand 1 (PD-L1), also known as cluster of differentiation 274 (CD274) or B7 homolog 1 (B7-H1), are being used for cancer treatments and in other clinical applications. PD-L1 is a 40 kDa type 1 transmembrane protein believed to play a major role in suppressing the immune system during particular events such as pregnancy, tissue allografts, autoimmune disease and other disease states such as hepatitis. The binding of PD-L1 to PD-1 or B7.1 transmits an inhibitory signal which reduces the proliferation of CD8+ T cells at the lymph nodes and supplementary to that PD-1 is also able to control the accumulation of foreign antigen specific T cells in the lymph nodes through apoptosis which is further mediated by a lower regulation of the gene Bcl-2.

In addition to treatment of cancers, PD-L1 inhibition has also shown promises in treating infectious diseases. In a mouse model of intracellular infection, L. monocytogenes induced PD-L1 protein expression in T cells, NK cells, and macrophages. PD-L1 blockade (e.g., using blocking antibodies) resulted in increased mortality for infected mice. Blockade reduced TNFα and nitric oxide production by macrophages, reduced granzyme B production by NK cells, and decreased proliferation of L. monocytogenes antigen-specific CD8 T cells (but not CD4 T cells). This evidence suggests that PD-L1 acts as a positive costimulatory molecule in intracellular infection.

### SUMMARY OF THE INVENTION

The present disclosure provides new single domain antibodies targeting the human PD-L1 protein. These single domain antibodies, despite their small sizes, exhibited superior binding affinity and biological functions. When included in various different formats of bispecific antibodies, some of the resulting bispecific antibodies exhibited excellent properties.

In one aspect provided is a single domain antibody or a polypeptide comprising the single domain antibody, wherein the single domain antibody has binding specificity to the human PD-L1 protein and comprises a complementarity determining region 1 (CDR1), a CDR2 and a CDR3 of anyone selected from the group consisting of SEQ ID NO: 1-36, 114-122 and 123-130. In some embodiments, the single domain antibody has binding specificity to the human PD-L1 protein and comprises a complementarity determining region 1 (CDR1), a CDR2 and a CDR3 of anyone selected from the group consisting of SEQ ID NO: 1-36, 114-122 and 123-130, and the CDR1, CDR2, and CDR3 are according to Kabat numbering scheme.

In some embodiments of the antibody or the polypeptide, the CDR1, CDR2 and CDR3 comprise: (1) the amino acid sequences of SEQ ID NO: 55, 56 and 57, respectively; or (2) the amino acid sequences of SEQ ID NO: 113, 49 and 50, respectively.

In some embodiments, the CDR1 comprises the amino acid sequence of SEQ ID NO: 55, the CDR2 comprises the amino acid sequence of SEQ ID NO: 56, and the CDR3 comprises the amino acid sequence of SEQ ID NO: 57. In some embodiments, the antibody is humanized. In some embodiments, the humanized antibody comprises one or more back mutations selected from the group consisting of 37F, 47F, 49A, 78V and 94A, according to Kabat numbering. In some embodiments, the humanized antibody comprises back mutations of 37F, 47F, 49A, 78V and 94A, according to Kabat numbering. In some embodiments, the antibody comprises an amino acid sequence of anyone selected from the group consisting of SEQ ID NO: 114-122. In some embodiments, the antibody comprises the amino acid sequence of SEQ ID NO: 119.

In some embodiments, the CDR1 comprises the amino acid sequence of SEQ ID NO: 113, the CDR2 comprises the amino acid sequence of SEQ ID NO: 49, and the CDR3 comprises the amino acid sequence of SEQ ID NO: 50. In some embodiments, the antibody is humanized. In some embodiments, the humanized antibody comprises one or more back mutations selected from the group consisting of 37Y, 44Q, 45R, 49A, 68A, 93R, and 94V, according to Kabat numbering. In some embodiments, the humanized antibody comprises back mutations of 37Y, 44Q, 45R, 49A, 68A, 93R, and 94V, according to Kabat numbering. In some embodiments, the antibody comprises an amino acid sequence of anyone selected from the group consisting of SEQ ID NO: 123-130. In some embodiments, the antibody comprises the amino acid sequence of SEQ ID NO: 127 or 130.

In some embodiments, the polypeptide is a bispecific antibody having a binding specificity to an antigen different from PD-L1.

In another aspect, provided herein is a bispecific antibody comprising the antibody of the present application and a second antibody or antigen-binding fragment having binding specificity to a target antigen that is not PD-L1.

In another aspect, provided herein is a bispecific antibody comprising an anti-PD-L1 portion having binding specificity to the human PD-L1 protein and an anti-TIGIT portion having binding specificity to the human TIGIT protein, wherein the anti-TIGIT portion comprises a heavy chain variable region (VH) comprising a VH CDR1, VH CDR2 and VH CDR3 of SEQ ID NO: 171, and a light chain variable region (VL) comprising a VL CDR1, VL CDR2 and VL CDR3 of SEQ ID NO: 172.

In some embodiments of the bispecific antibody, the anti-TIGIT portion comprises a heavy chain variable region (VH) comprising a VH CDR1, VH CDR2 and VH CDR3 of SEQ ID NO: 171, and a light chain variable region (VL) comprising a VL CDR1, VL CDR2 and VL CDR3 of SEQ ID NO: 172, and the VH CDR1, VH CDR2, VH CDR3, VL CDR1, VL CDR2 and VL CDR3 are according to Kabat numbering scheme. In some embodiments, the anti-TIGIT portion comprises a heavy chain variable region (VH) comprising a VH CDR1, VH CDR2 and VH CDR3, and a light chain variable region (VL) comprising a VL CDR1, VL CDR2 and VL CDR3, wherein the VH CDR1, VH CDR1, VH CDR3, VL CDR1, VL CDR2 and VL CDR3 comprise the amino acid sequences of SEQ ID NO: 173-178, respectively. In some embodiments, the anti-TIGIT portion comprises a VH comprising an amino acid sequence of SEQ ID NO: 171, and VL comprising an amino acid sequence of SEQ ID NO: 172.

In some embodiments of the bispecific antibody, the anti-PD-L1 antigen-binding portion comprises a full-length antibody, a Fab, a F(ab′)2, a scFv, a scFv-Fc, or a single domain antibody. In some embodiments, the anti-PD-L1 antigen-binding portion comprises a single domain antibody. In some embodiments, the single domain antibody comprises a CDR1, a CDR2 and a CDR3 of anyone selected from the group consisting of SEQ ID NO: 1-36, 114-122 and 123-130. In some embodiments, the single domain antibody comprises a CDR1, a CDR2 and a CDR3 of anyone selected from the group consisting of SEQ ID NO: 1-36, 114-122 and 123-130, and the CDR1, CDR2, and CDR3 are according to Kabat numbering scheme. In some embodiments, the anti-PD-L1 portion comprises a single domain antibody comprising a complementarity determining region 1 (CDR1), a CDR2 and a CDR3, wherein the CDR1, CDR2 and CDR3 comprise: (1) the amino acid sequences of SEQ ID NO: 55, 56 and 57, respectively; or (2) the amino acid sequences of SEQ ID NO: 113, 49 and 50, respectively.

In some embodiments of the bispecific antibody, the anti-PD-L1 portion comprises a CDR1 comprising the amino acid sequence of SEQ ID NO: 55, a CDR2 comprising the amino acid sequence of SEQ ID NO: 56, and a CDR3 comprising the amino acid sequence of SEQ ID NO: 57. In some embodiments, the anti-PD-L1 portion comprises a CDR1 comprising the amino acid sequence of SEQ ID NO: 113, a CDR2 comprising the amino acid sequence of SEQ ID NO: 49, and a CDR3 comprising the amino acid sequence of SEQ ID NO: 50.

In some embodiments of the bispecific antibody, the anti-PD-L1 portion is humanized. In some embodiments, the anti-PD-L1 portion comprises an amino acid sequence selected from the group consisting of SEQ ID NO: 114-122 and 123-130. In some embodiments, the anti-PD-L1 portion comprises an amino acid sequence of 119 or 130.

In some embodiments of the bispecific antibody, the anti-PD-L1 portion is fused to the C-terminal of heavy chain of the anti-TIGIT portion. In some embodiments, the anti-PD-L1 portion is fused to the N-terminal of heavy chain of the anti-TIGIT portion. In some embodiments, the anti-PD-L1 portion is fused to the C-terminal of a light chain of the anti-TIGIT portion. In some embodiments, the anti-PD-L1 portion is fused to the N-terminal of light chain of the anti-TIGIT portion.

In some embodiments of the bispecific antibody, the bispecific antibody is a homodimer. In some embodiments, the bispecific antibody includes two of the anti-PD-L1 portions. In some embodiments, each of the two of the anti-PD-L1 portions is fused to the C-terminal of the heavy chain of the anti-TIGIT portion. In some embodiments, the bispecific antibody includes four of the anti-PD-L1 portions.

In some embodiments of the bispecific antibody, the bispecific antibody comprises: (1) a heavy component comprising an amino acid sequence of anyone selected from the group consisting of SEQ ID NO: 179, 181, 182, and 184, and (2) a light component comprising an amino acid sequence of anyone selected from the group consisting of SEQ ID NO: 180 and 183. In some embodiments, the bispecific antibody comprises a heavy component comprising an amino acid sequence of SEQ IN NO: 179 and a light component comprising an amino acid sequence of SEQ ID NO: 180. In some embodiments, the bispecific antibody comprises a heavy component comprising an amino acid sequence of SEQ IN NO: 184 and a light component comprising an amino acid sequence of SEQ ID NO: 180.

In another aspect, provided herein is a bispecific antibody comprising an anti-PD-L1 portion having binding specificity to the human PD-L1 protein and an anti-CD47 portion having binding specificity to the human CD47 protein, wherein the anti-CD47 portion comprises a heavy chain variable region (VH) comprising a VH CDR1, VH CDR2 and VH CDR3 of SEQ ID NO: 131 or 133, and a light chain variable region (VL) comprising a VL CDR1, VL CDR2 and VL CDR3 of SEQ ID NO: 132 or 134.

In some embodiments of the bispecific antibody, the anti-CD47 portion comprises a heavy chain variable region (VH) comprising a VH CDR1, VH CDR2 and VH CDR3 of SEQ ID NO: 131 or 133, and a light chain variable region (VL) comprising a VL CDR1, VL CDR2 and VL CDR3 of SEQ ID NO: 132 or 134, and the VH CDR1, VH CDR2, VH CDR3, VL CDR1, VL CDR2 and VL CDR3 are according to Kabat numbering scheme.

In some embodiments, the anti-CD47 portion comprises a heavy chain variable region (VH) comprising a VH CDR1, VH CDR2 and VH CDR3, and a light chain variable region (VL) comprising a VL CDR1, VL CDR2 and VL CDR3, wherein the VH CDR1, VH CDR1, VH CDR3, VL CDR1, VL CDR2 and VL CDR3 comprise the amino acid sequences of SEQ ID NO: 135-140, respectively. In some embodiments, the anti-CD47 portion comprises a VH comprising an amino acid sequence of SEQ ID NO: 131, and VL comprising an amino acid sequence of SEQ ID NO: 132.

In some embodiments, the anti-CD47 portion comprises a heavy chain variable region (VH) comprising a VH CDR1, VH CDR2 and VH CDR3, and a light chain variable region (VL) comprising a VL CDR1, VL CDR2 and VL CDR3, wherein the VH CDR1, VH CDR1, VH CDR3, VL CDR1, VL CDR2 and VL CDR3 comprise the amino acid sequences of SEQ ID NO: 141-146, respectively. In some embodiments, the anti-CD47 portion comprises a VH comprising an amino acid sequence of SEQ ID NO: 133, and VL comprising an amino acid sequence of SEQ ID NO: 134.

In some embodiments of the bispecific antibody, the anti-PD-L1 antigen-binding portion comprises a full-length antibody, a Fab, a F(ab′)2, a scFv, a scFv-Fc, or a single domain antibody. In some embodiments, the anti-PD-L1 antigen-binding portion comprises a single domain antibody. In some embodiments, the single domain antibody comprises a CDR1, a CDR2 and a CDR3 of anyone selected from the group consisting of SEQ ID NO: 1-36, 114-122 and 123-130. In some embodiments, the single domain antibody comprises a CDR1, a CDR2 and a CDR3 of anyone selected from the group consisting of SEQ ID NO: 1-36, 114-122 and 123-130, and the CDR1, CDR2, and CDR3 are according to Kabat numbering scheme. In some embodiments, the anti-PD-L1 portion comprises a single domain antibody comprising a complementarity determining region 1 (CDR1), a CDR2 and a CDR3, wherein the CDR1, CDR2 and CDR3 comprise: (1) the amino acid sequences of SEQ ID NO: 55, 56 and 57, respectively; or (2) the amino acid sequences of SEQ ID NO: 113, 49 and 50, respectively.

In some embodiments of the bispecific antibody, the anti-PD-L1 portion comprises a CDR1 comprising the amino acid sequence of SEQ ID NO: 55, a CDR2 comprising the amino acid sequence of SEQ ID NO: 56, and a CDR3 comprising the amino acid sequence of SEQ ID NO: 57. In some embodiments, the anti-PD-L1 portion comprises a CDR1 comprising the amino acid sequence of SEQ ID NO: 113, a CDR2 comprising the amino acid sequence of SEQ ID NO: 49, and a CDR3 comprising the amino acid sequence of SEQ ID NO: 50.

In some embodiments of the bispecific antibody, the anti-PD-L1 portion is humanized. In some embodiments, the anti-PD-L1 portion comprises an amino acid sequence selected from the group consisting of SEQ ID NO: 114-122 and 123-130. In some embodiments, the anti-PD-L1 portion comprises an amino acid sequence of 119 or 130.

In some embodiments of the bispecific antibody, the anti-PD-L1 portion is fused to the C-terminal of heavy chain of the anti-CD47 portion. In some embodiments, the anti-PD-L1 portion is fused to the N-terminal of heavy chain of the anti-CD47 portion. In some embodiments, the anti-PD-L1 portion is fused to the C-terminal of a light chain of the anti-CD47 portion. In some embodiments, the anti-PD-L1 portion is fused to the N-terminal of light chain of the anti-CD47 portion.

In some embodiments of the bispecific antibody, the bispecific antibody is a homodimer. In some embodiments, the bispecific antibody includes two of the anti-PD-L1 portions. In some embodiments, each of the two of the anti-PD-L1 portions is fused to the C-terminal of the heavy chain of the anti-CD47 portion. In some embodiments, the bispecific antibody includes four of the anti-PD-L1 portions.

In some embodiments of the bispecific antibody, the bispecific antibody comprises: (1) a heavy component comprising an amino acid sequence of anyone selected from the group consisting of SEQ ID NO: 147, 149, 151, 153, 155, 157, 159, 161, 163, 164, 166, 167, 169, and 170, and (2) a light component comprising an amino acid sequence of anyone selected from the group consisting of SEQ ID NO: 148, 150, 152, 154, 156, 158, 160, 162, 165, and 168.

In another aspect, provided herein is a polynucleotide encoding the antibody or polypeptide of the present application, or the bispecific antibody of the present application.

In another aspect, provided herein is a vector comprising the polynucleotide of the present application.

In another aspect, provided is a cell comprising the polynucleotide or the vector of the present application.

In another aspect, provided herein is a composition comprising: (1) the antibody or polypeptide, the bispecific antibody, or the polynucleotide of the present application, and (2) a pharmaceutically acceptable carrier.

In another aspect, provided herein is a method of treating cancer in a patient in need thereof, comprising administering to the patient an effective amount of the antibody or polypeptide, the bispecific antibody, or the polynucleotide of the present application. In another aspect, provided herein is use of the antibody or polypeptide, the bispecific antibody, or the polynucleotide of the present application for the preparation of a medicament for treating cancer.

In some embodiments, the cancer is a solid tumor. In some embodiments, the cancer is selected from the group consisting of bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, oesophageal cancer, ovarian cancer, renal cancer, melanoma, prostate cancer and thyroid cancer.

### BRIEF DESCRIPTION OF THE DRAWINGS

FIG. 1A-IC: FIG. 1A illustrates the exemplary anti-PD-L1 antibodies of the present application effectively blocked the interaction between PD-1 and PD-L1, FIG. 1B illustrates specific binding of the exemplary anti-PD-L1 antibodies of the present application with human PD-L1, and FIG. 1C illustrates specific binding of the exemplary anti-PD-L1 antibodies of the present application with Raji cells overexpressing human PD-L1.

FIG. 2A-2C illustrate blocking of PD-1/PD-L1 interaction by the exemplary anti-PD-L1 antibodies of the present application could enhance NFAT-mediated luciferase activity in a dose dependent manner.

FIG. 3A-3F illustrates exemplary formats of the anti-CD47/PD-L1 bispecific antibodies of the present application.

FIGS. 4A and 4B illustrates the exemplary anti-CD47/PD-L1 bispecific antibodies of the present application blocked CD47/SIRPα interaction in a dose dependent manner.

FIGS. 5A and 5B illustrates the exemplary anti-CD47/PD-L1 bispecific antibodies of the present application effectively blocked PD-1/PD-L1 mediated NF-AT-luciferase activity.

FIG. 6A-6C illustrates ADCP efficacy of the exemplary anti-CD47/PD-L1 bispecific antibodies of the present application.

FIGS. 7A and 7B illustrates RKO binding capability of the exemplary antibodies of the present application.

FIG. 8A-8C: FIGS. 8A and 8B illustrate the exemplary anti-CD47/PD-L1 bispecific antibodies of the present application displayed minimal or no RBC binding, and FIG. 8C illustrates in vivo anti-tumor efficacy of the exemplary anti-CD47/PD-L1 bispecific antibodies of the present application.

FIG. 9 illustrates exemplary formats of anti-TIGIT/PD-L1 bispecific antibodies of the present application.

FIG. 10A-10C illustrate binding properties of the exemplary anti-TIGIT/PD-L1 bsAbs of the present application with human PD-L1 protein.

FIG. 11 illustrates blocking of on PD-1/PD-L1 interaction by the exemplary anti-TIGIT/PD-L1 bsAbs of the present application could enhance NFAT-mediated luciferase activity in a dose dependent manner.

FIGS. 12A and 12B illustrate specific binding of the exemplary anti-TIGIT/PD-L1 bsAbs of the present application with human TIGIT protein.

FIG. 13 illustrates effective blocking of TIGIT/CD155 interaction by the exemplary anti-TIGIT/PD-L1 bsAbs of the present application could enhance NFAT-mediated luciferase activity in a dose dependent manner.

FIG. 14 illustrates antagonistic activity of the exemplary anti-TIGIT/PD-L1 bsAbs of the present application in Jurkat cells based bifunctional assay.

FIG. 15 illustrates exemplary anti-TIGIT/PD-L1 bsAbs of the present application significantly enhanced IFN-γ production of human primary CD8+ T cells in a concentration-dependent manner.

### DETAILED DESCRIPTION

### Definitions

It is to be noted that the term “a” or “an” entity refers to one or more of that entity; for example, “an antibody” is understood to represent one or more antibodies. As such, the terms “a” (or “an”), “one or more”, and “at least one” can be used interchangeably herein.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology.

Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Biologically equivalent polynucleotides are those having the above-noted specified percent homology and encoding a polypeptide having the same or similar biological activity.

The term “an equivalent nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference polypeptide or polynucleotide. In some aspects, the equivalent sequence retains the activity (e.g., epitope-binding) or structure (e.g., salt-bridge) of the reference sequence.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

A “single-chain variable fragment” or “scFv” refers to a fusion protein of the variable regions of the heavy (V_{H}) and light chains (V_{L}) of immunoglobulins. In some aspects, the regions are connected with a short linker peptide of ten to about 25 amino acids. The linker can be rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker. ScFv molecules are known in the art and are described, e.g., in U.S. Pat. No. 5,892,019.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG1, IgG2, IgG3, IgG4, IgG5, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

By “specifically binds” or “has specificity to”, it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D”.

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal”, is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### Single Domain Anti-PD-L1 Antibodies

The present disclosure provides single chain anti-PD-L1 antibodies with high affinity to the human PD-L1 protein. The antibodies exhibited potent binding and inhibitory activities and are useful for therapeutic and diagnostics uses. Also importantly, when incorporated as one of the targeting units in a variety of different formats of bispecific antibodies, certain resulting bispecific antibodies exhibited outstanding properties, establishing the additional utility of these single domain anti-PD-L1 antibodies.

Accordingly, in one embodiment of the present disclosure, provided are single domain antibodies and polypeptides that include such a single domain antibody. In some embodiments, the polypeptide is a bispecific antibody, a tri-specific antibody, or a multi-specific antibody.

In some embodiments, wherein the single domain antibody has binding specificity to the human PD-L1 protein and comprises a complementarity determining region 1 (CDR1), a CDR2 and a CDR3. In some embodiments, the CDR1, CDR2 and CDR3 include the amino acid sequences of SEQ ID NO: 55, 56 and 57, respectively. In some embodiments, the CDR1, CDR2 and CDR3 include the amino acid sequences of SEQ ID NO: 113, 49 and 50, respectively. In some embodiments, the CDR1, CDR2 and CDR3 include CDR1, CDR2 and CDR3, respectively, of any one of the antibodies provided in Table 1 (e.g., SEQ ID NO: 1-36).

In one embodiment, in the antibody, the CDR1 includes the amino acid sequence of SEQ ID NO: 55, the CDR2 includes the amino acid sequence of SEQ ID NO: 56, and the CDR3 includes the amino acid sequence of SEQ ID NO: 57. SEQ ID NO: 55, 56 and 57 are the CDRs of antibody ALP-Tan-3p-93, and its humanized counterparts 93_VH-1 through 93_VH-9. In some embodiments, the CDR1, CDR2 and CDR3 include SEQ ID NO: 55, 56 and 57 but with one, two, or three amino acid additions, deletions, and/or substitutions, respectively. In some embodiments, the substitutions are conservative substitutions.

In some embodiments, the antibody is humanized. In some embodiments, the humanized antibody includes one or more back mutations tested to improve the properties of the grafted antibody. In some embodiments, the back mutations are selected from the group consisting of 37F, 47F, 49A, 78V and 94A, according to Kabat numbering. In some embodiments, the humanized antibody includes all of back mutations of 37F, 47F, 49A, 78V and 94A, according to Kabat numbering.

Example humanized antibodies include 93_VH-1, 93_VH-2, 93_VH-3, 93_VH-4, 93_VH-5, 93_VH-6, 93_VH-7, 93_VH-8, and 93_VH-9. Example sequences are SEQ ID NO: 114-122. In some embodiments, the humanized antibody has the amino acid sequence of SEQ ID NO: 119.

In some embodiments, in the antibody, the CDR1 includes the amino acid sequence of SEQ ID NO: 113, the CDR2 includes the amino acid sequence of SEQ ID NO: 49, and the CDR3 includes the amino acid sequence of SEQ ID NO: 50. SEQ ID NO: 113, 49 and 50 are the CDRs of humanized antibodies 112-VHH1-PTM, 112-VHH2-PTM, 112-VHH3-PTM, 112-VHH4-PTM, 112-VHH5-PTM, 112-VHH6-PTM, or 112-VHH7-PTM. Compared to the original antibody ALP-Tan-3p-112, these humanized antibodies included a N34Q substitution (Kabat numbering) in CDR1 (SEQ ID NO: 48), to prevent posttranslational modification. In some embodiments, the CDR1, CDR2 and CDR3 include SEQ ID NO: 113, 49 and 50 but with one, two, or three amino acid additions, deletions, and/or substitutions, respectively. In some embodiments, the substitutions are conservative substitutions.

In some embodiments, the antibody is humanized. In some embodiments, the humanized antibody includes one or more back mutations selected from the group consisting of 37Y, 44Q, 45R, 49A, 68A, 93R, and 94V, according to Kabat numbering. In some embodiments, the humanized antibody includes all of back mutations of 37Y, 44Q, 45R, 49A, 68A, 93R, and 94V, according to Kabat numbering. In some embodiments, the antibody comprises an amino acid sequence selected from the group consisting of SEQ ID NO: 123-130. In some embodiments, the antibody comprises the amino acid sequence of SEQ ID NO: 127. In some embodiments, the antibody comprises the amino acid sequence of SEQ ID NO: 130.

In some embodiments, in the antibody, the CDR1, CDR2 and CDR3 include CDR1, CDR2 and CDR3, respectively, of any one of the antibodies provided in Table 1 (e.g., SEQ ID NO: 1-36). In one embodiment, the CDR1, CDR2 and CDR3 include the amino acid sequences of SEQ ID NO: 37, 38, and 39, respectively. In one embodiment, the CDR1, CDR2 and CDR3 include the amino acid sequences of SEQ ID NO: 40, 41, and 42, respectively. In one embodiment, the CDR1, CDR2 and CDR3 include the amino acid sequences of SEQ ID NO: 43, 44, and 45, respectively. In one embodiment, the CDR1, CDR2 and CDR3 include the amino acid sequences of SEQ ID NO: 40, 46, and 47, respectively.

In one embodiment, the CDR1, CDR2 and CDR3 include the amino acid sequences of SEQ ID NO: 40, 46, and 47, respectively. In one embodiment, the CDR1, CDR2 and CDR3 include the amino acid sequences of SEQ ID NO: 48, 49, and 50, respectively. In one embodiment, the CDR1, CDR2 and CDR3 include the amino acid sequences of SEQ ID NO: 40, 51, and 52, respectively. In one embodiment, the CDR1, CDR2 and CDR3 include the amino acid sequences of SEQ ID NO: 37, 53, and 54, respectively. In one embodiment, the CDR1, CDR2 and CDR3 include the amino acid sequences of SEQ ID NO: 40, 41, and 47, respectively.

In one embodiment, the CDR1, CDR2 and CDR3 include the amino acid sequences of SEQ ID NO: 40, 46, and 47, respectively. In one embodiment, the CDR1, CDR2 and CDR3 include the amino acid sequences of SEQ ID NO: 55, 56, and 57, respectively. In one embodiment, the CDR1, CDR2 and CDR3 include the amino acid sequences of SEQ ID NO: 40, 41, and 58, respectively. In one embodiment, the CDR1, CDR2 and CDR3 include the amino acid sequences of SEQ ID NO: 37, 53, and 39, respectively. In one embodiment, the CDR1, CDR2 and CDR3 include the amino acid sequences of SEQ ID NO: 37, 59, and 60, respectively.

In one embodiment, the CDR1, CDR2 and CDR3 include the amino acid sequences of SEQ ID NO: 61, 62, and 63, respectively. In one embodiment, the CDR1, CDR2 and CDR3 include the amino acid sequences of SEQ ID NO: 64, 65, and 66, respectively. In one embodiment, the CDR1, CDR2 and CDR3 include the amino acid sequences of SEQ ID NO: 67, 68, and 69, respectively. In one embodiment, the CDR1, CDR2 and CDR3 include the amino acid sequences of SEQ ID NO: 70, 71, and 72, respectively. In one embodiment, the CDR1, CDR2 and CDR3 include the amino acid sequences of SEQ ID NO: 37, 73, and 74, respectively.

In one embodiment, the CDR1, CDR2 and CDR3 include the amino acid sequences of SEQ ID NO: 37, 75, and 76, respectively. In one embodiment, the CDR1, CDR2 and CDR3 include the amino acid sequences of SEQ ID NO: 64, 77, and 66, respectively. In one embodiment, the CDR1, CDR2 and CDR3 include the amino acid sequences of SEQ ID NO: 37, 78, and 79, respectively. In one embodiment, the CDR1, CDR2 and CDR3 include the amino acid sequences of SEQ ID NO: 80, 81, and 82, respectively. In one embodiment, the CDR1, CDR2 and CDR3 include the amino acid sequences of SEQ ID NO: 40, 83, and 47, respectively.

In one embodiment, the CDR1, CDR2 and CDR3 include the amino acid sequences of SEQ ID NO: 84, 85, and 86, respectively. In one embodiment, the CDR1, CDR2 and CDR3 include the amino acid sequences of SEQ ID NO: 64, 87, and 66, respectively. In one embodiment, the CDR1, CDR2 and CDR3 include the amino acid sequences of SEQ ID NO: 84, 88, and 89, respectively. In one embodiment, the CDR1, CDR2 and CDR3 include the amino acid sequences of SEQ ID NO: 84, 90, and 91, respectively. In one embodiment, the CDR1, CDR2 and CDR3 include the amino acid sequences of SEQ ID NO: 92, 93, and 94, respectively. In one embodiment, the CDR1, CDR2 and CDR3 include the amino acid sequences of SEQ ID NO: 95, 96, and 97, respectively. In one embodiment, the CDR1, CDR2 and CDR3 include the amino acid sequences of SEQ ID NO: 84, 98, and 99, respectively.

In one embodiment, the CDR1, CDR2 and CDR3 include the amino acid sequences of SEQ ID NO: 100, 101, and 102, respectively. In one embodiment, the CDR1, CDR2 and CDR3 include the amino acid sequences of SEQ ID NO: 103, 104, and 105, respectively. In one embodiment, the CDR1, CDR2 and CDR3 include the amino acid sequences of SEQ ID NO: 84, 85, and 106, respectively. In one embodiment, the CDR1, CDR2 and CDR3 include the amino acid sequences of SEQ ID NO: 107, 108, and 109, respectively. In one embodiment, the CDR1, CDR2 and CDR3 include the amino acid sequences of SEQ ID NO: 110, 111, and 112, respectively.

In some embodiments, the antibody includes an amino acid sequence selected from SEQ ID NO: 1-36.

Also provided, in some embodiments, are anti-PD-L1 antibodies and antigen binding fragments that compete with any of the antibodies disclosed herein in binding to human PD-L1. Also provided, in some embodiments, are anti-PD-L1 antibodies and antigen binding fragments that bind to the same epitope as any of the antibodies disclosed herein. Also provided, in some embodiments, are anti-PD-L1 antibodies and antigen binding fragments that included the VH CDR1, CDR2, and CDR3 and VL CDR1, CDR2 and CDR3 of the antibodies disclosed herein.

Also provided are compositions that include the antibody or the polypeptide and a pharmaceutically acceptable carrier.

It will also be understood by one of ordinary skill in the art that antibodies as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, 98%, or 99% identical to the starting sequence. In some embodiments, the modified antibody or fragment retains the designate CDR sequences.

Also provided are bispecific and multispecific antibodies that includes one, two, three or four units of the single domain anti-PD-L1 antibody as disclosed herein, and one or more other specificities (not PD-L1).

The present disclosure provides bi- and multi-specific antibodies that have binding specificities at least to the human PD-L1 and CD47 proteins. PD-L1 is a critical “don't find me” signal to the adaptive immune system, whereas CD47 transmits an anti-phagocytic “don't eat me” signal to the innate immune system. They are often overexpressed on human tumors. Thus, dual targeting both innate and adaptive immune checkpoints would likely maximize anti-tumor therapeutic effect and elicit more durable responses.

In some embodiments, the bi- and multi-specific antibodies include an anti-PD-L1 portion which includes at least a single domain anti-PD-L1 antibody. As demonstrated, the single chain anti-PD-L1 antibodies have high affinity to the human PD-L1 protein. The antibodies exhibited potent binding and inhibitory activities and are useful for therapeutic and diagnostics uses.

In some embodiments, wherein the single domain antibody has binding specificity to the human PD-L1 protein and comprises a complementarity determining region 1 (CDR1), a CDR2 and a CDR3. In some embodiments, the CDR1, CDR2 and CDR3 include the amino acid sequences of SEQ ID NO: 55, 56 and 57, respectively. In some embodiments, the CDR1, CDR2 and CDR3 include the amino acid sequences of SEQ ID NO: 113, 49 and 50, respectively. In some embodiments, the CDR1, CDR2 and CDR3 include CDR1, CDR2 and CDR3, respectively, of any one of the antibodies provided in Table 1 (e.g., SEQ ID NO: 1-36).

In one embodiment, in the antibody, the CDR1 includes the amino acid sequence of SEQ ID NO: 55, the CDR2 includes the amino acid sequence of SEQ ID NO: 56, and the CDR3 includes the amino acid sequence of SEQ ID NO: 57. SEQ ID NO: 55, 56 and 57 are the CDRs of antibody ALP-Tan-3p-93, and its humanized counterparts 93_VH-1 through 93_VH-9. In some embodiments, the CDR1, CDR2 and CDR3 include SEQ ID NO: 55, 56 and 57 but with one, two, or three amino acid additions, deletions, and/or substitutions, respectively. In some embodiments, the substitutions are conservative substitutions.

In some embodiments, the antibody is humanized. In some embodiments, the humanized antibody includes one or more back mutations tested to improve the properties of the grafted antibody. In some embodiments, the back mutations are selected from the group consisting of 37F, 47F, 49A, 78V and 94A, according to Kabat numbering. In some embodiments, the humanized antibody includes all of back mutations of 37F, 47F, 49A, 78V and 94A, according to Kabat numbering.

Example humanized antibodies include 93_VH-1, 93_VH-2, 93_VH-3, 93_VH-4, 93_VH-5, 93_VH-6, 93_VH-7, 93_VH-8, and 93_VH-9. Example sequences are SEQ ID NO: 114-122. In some embodiments, the humanized antibody has the amino acid sequence of SEQ ID NO: 119.

In some embodiments, in the antibody, the CDR1 includes the amino acid sequence of SEQ ID NO: 113, the CDR2 includes the amino acid sequence of SEQ ID NO: 49, and the CDR3 includes the amino acid sequence of SEQ ID NO: 50. SEQ ID NO: 113, 49 and 50 are the CDRs of humanized antibodies 112-VHH1-PTM, 112-VHH2-PTM, 112-VHH3-PTM, 112-VHH4-PTM, 112-VHH5-PTM, 112-VHH6-PTM, or 112-VHH7-PTM. Compared to the original antibody ALP-Tan-3p-112, these humanized antibodies included a N34Q substitution (Kabat numbering) in CDR1 (SEQ ID NO: 48), to prevent posttranslational modification. In some embodiments, the CDR1, CDR2 and CDR3 include SEQ ID NO: 113, 49 and 50 but with one, two, or three amino acid additions, deletions, and/or substitutions, respectively. In some embodiments, the substitutions are conservative substitutions.

In some embodiments, the antibody is humanized. In some embodiments, the humanized antibody includes one or more back mutations selected from the group consisting of 37Y, 44Q, 45R, 49A, 68A, 93R, and 94V, according to Kabat numbering. In some embodiments, the humanized antibody includes all of back mutations of 37Y, 44Q, 45R, 49A, 68A, 93R, and 94V, according to Kabat numbering. In some embodiments, the antibody comprises an amino acid sequence selected from the group consisting of SEQ ID NO: 123-130. In some embodiments, the antibody comprises the amino acid sequence of SEQ ID NO: 127. In some embodiments, the antibody comprises the amino acid sequence of SEQ ID NO: 130.

In some embodiments, in the antibody, the CDR1, CDR2 and CDR3 include CDR1, CDR2 and CDR3, respectively, of any one of the antibodies provided in Table 1 (e.g., SEQ ID NO: 1-36). In one embodiment, the CDR1, CDR2 and CDR3 include the amino acid sequences of SEQ ID NO: 37, 38, and 39, respectively. In one embodiment, the CDR1, CDR2 and CDR3 include the amino acid sequences of SEQ ID NO: 40, 41, and 42, respectively. In one embodiment, the CDR1, CDR2 and CDR3 include the amino acid sequences of SEQ ID NO: 43, 44, and 45, respectively. In one embodiment, the CDR1, CDR2 and CDR3 include the amino acid sequences of SEQ ID NO: 40, 46, and 47, respectively.

In one embodiment, the CDR1, CDR2 and CDR3 include the amino acid sequences of SEQ ID NO: 40, 46, and 47, respectively. In one embodiment, the CDR1, CDR2 and CDR3 include the amino acid sequences of SEQ ID NO: 48, 49, and 50, respectively. In one embodiment, the CDR1, CDR2 and CDR3 include the amino acid sequences of SEQ ID NO: 40, 51, and 52, respectively. In one embodiment, the CDR1, CDR2 and CDR3 include the amino acid sequences of SEQ ID NO: 37, 53, and 54, respectively. In one embodiment, the CDR1, CDR2 and CDR3 include the amino acid sequences of SEQ ID NO: 40, 41, and 47, respectively.

In one embodiment, the CDR1, CDR2 and CDR3 include the amino acid sequences of SEQ ID NO: 40, 46, and 47, respectively. In one embodiment, the CDR1, CDR2 and CDR3 include the amino acid sequences of SEQ ID NO: 55, 56, and 57, respectively. In one embodiment, the CDR1, CDR2 and CDR3 include the amino acid sequences of SEQ ID NO: 40, 41, and 58, respectively. In one embodiment, the CDR1, CDR2 and CDR3 include the amino acid sequences of SEQ ID NO: 37, 53, and 39, respectively. In one embodiment, the CDR1, CDR2 and CDR3 include the amino acid sequences of SEQ ID NO: 37, 59, and 60, respectively.

In one embodiment, the CDR1, CDR2 and CDR3 include the amino acid sequences of SEQ ID NO: 61, 62, and 63, respectively. In one embodiment, the CDR1, CDR2 and CDR3 include the amino acid sequences of SEQ ID NO: 64, 65, and 66, respectively. In one embodiment, the CDR1, CDR2 and CDR3 include the amino acid sequences of SEQ ID NO: 67, 68, and 69, respectively. In one embodiment, the CDR1, CDR2 and CDR3 include the amino acid sequences of SEQ ID NO: 70, 71, and 72, respectively. In one embodiment, the CDR1, CDR2 and CDR3 include the amino acid sequences of SEQ ID NO: 37, 73, and 74, respectively.

In one embodiment, the CDR1, CDR2 and CDR3 include the amino acid sequences of SEQ ID NO: 37, 75, and 76, respectively. In one embodiment, the CDR1, CDR2 and CDR3 include the amino acid sequences of SEQ ID NO: 64, 77, and 66, respectively. In one embodiment, the CDR1, CDR2 and CDR3 include the amino acid sequences of SEQ ID NO: 37, 78, and 79, respectively. In one embodiment, the CDR1, CDR2 and CDR3 include the amino acid sequences of SEQ ID NO: 80, 81, and 82, respectively. In one embodiment, the CDR1, CDR2 and CDR3 include the amino acid sequences of SEQ ID NO: 40, 83, and 47, respectively.

In one embodiment, the CDR1, CDR2 and CDR3 include the amino acid sequences of SEQ ID NO: 84, 85, and 86, respectively. In one embodiment, the CDR1, CDR2 and CDR3 include the amino acid sequences of SEQ ID NO: 64, 87, and 66, respectively. In one embodiment, the CDR1, CDR2 and CDR3 include the amino acid sequences of SEQ ID NO: 84, 88, and 89, respectively. In one embodiment, the CDR1, CDR2 and CDR3 include the amino acid sequences of SEQ ID NO: 84, 90, and 91, respectively. In one embodiment, the CDR1, CDR2 and CDR3 include the amino acid sequences of SEQ ID NO: 92, 93, and 94, respectively. In one embodiment, the CDR1, CDR2 and CDR3 include the amino acid sequences of SEQ ID NO: 95, 96, and 97, respectively. In one embodiment, the CDR1, CDR2 and CDR3 include the amino acid sequences of SEQ ID NO: 84, 98, and 99, respectively.

In one embodiment, the CDR1, CDR2 and CDR3 include the amino acid sequences of SEQ ID NO: 100, 101, and 102, respectively. In one embodiment, the CDR1, CDR2 and CDR3 include the amino acid sequences of SEQ ID NO: 103, 104, and 105, respectively. In one embodiment, the CDR1, CDR2 and CDR3 include the amino acid sequences of SEQ ID NO: 84, 85, and 106, respectively. In one embodiment, the CDR1, CDR2 and CDR3 include the amino acid sequences of SEQ ID NO: 107, 108, and 109, respectively. In one embodiment, the CDR1, CDR2 and CDR3 include the amino acid sequences of SEQ ID NO: 110, 111, and 112, respectively.

In some embodiments, the antibody includes an amino acid sequence selected from SEQ ID NO: 1-36.

Also provided, in some embodiments, are anti-PD-L1 antibodies and antigen binding fragments that compete with any of the antibodies disclosed herein in binding to human PD-L1. Also provided, in some embodiments, are anti-PD-L1 antibodies and antigen binding fragments that bind to the same epitope as any of the antibodies disclosed herein. Also provided, in some embodiments, are anti-PD-L1 antibodies and antigen binding fragments that included the VH CDR1, CDR2, and CDR3 and VL CDR1, CDR2 and CDR3 of the antibodies disclosed herein.

In some embodiments, the anti-CD47 portion of the bi- or multi-specific antibodies has a pair (or, in some embodiments, two pairs) of heavy chain variable region (VH) and a light chain variable region (VL). The VH can include a VH CDR1, a VH CDR and a VH CDR3. The VL can include a VL CDR1, a VL CDR2 and a VL CDR3.

In some embodiments, the VH CDR1, VH CDR, VH CDR3, VL CDR1, VL CDR2 and VL CDR3 include the amino acid sequences of SEQ ID NO: 141-146, respectively. These CDRs are the ones from the parental anti-CD47 antibody 34C5. In some embodiments, the VH includes the amino acid sequence of SEQ ID NO: 133, and the VL includes the amino acid sequence of SEQ ID NO: 134 (Table 5).

In some embodiments, the VH CDR1, VH CDR, VH CDR3, VL CDR1, VL CDR2 and VL CDR3 include the amino acid sequences of SEQ ID NO: 135-140, respectively. These CDRs are the ones from the parental anti-CD47 antibody 13H3. In some embodiments, the VH includes the amino acid sequence of SEQ ID NO: 131, and the VL includes the amino acid sequence of SEQ ID NO: 132 (Table 5).

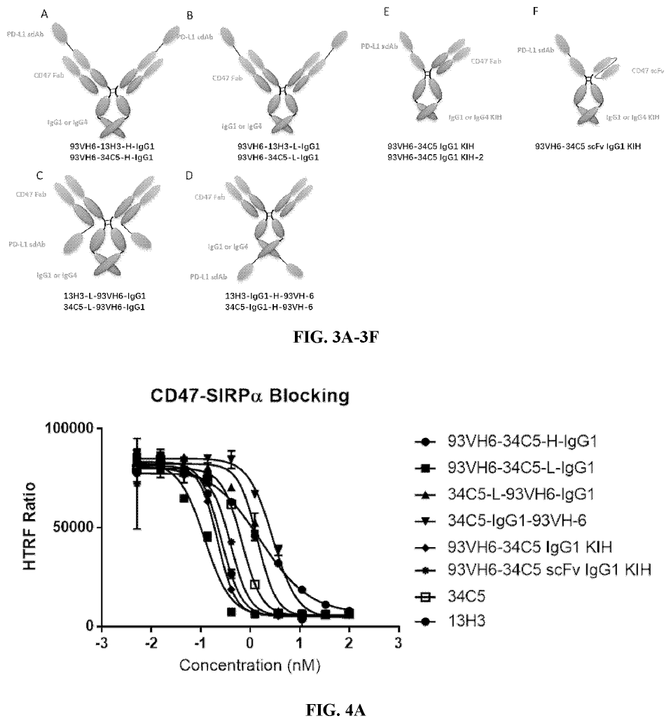

The bispecific antibody can take any format, including those illustrated in FIG. 3. In one embodiment, the bispecific antibody is symmetrical. An example is provided in FIG. 3A, in which two single domain anti-PD-L1 antibodies are fused, optionally through a linker, to the N-terminus of each of the heavy chains of the anti-CD47 antibody. In the example of FIG. 3B, the single domain anti-PD-L1 antibodies are fused, optionally through a linker, to the N-terminus of each of the light chains of the anti-CD47 antibody.

In the example of FIG. 3C, the single domain anti-PD-L1 antibodies are fused, optionally through a linker, to the C-terminus of each of the light chains (constant regions) of the anti-CD47 antibody. In the example of FIG. 3D, the single domain anti-PD-L1 antibodies are fused, optionally through a linker, to the C-terminus of the Fc portion of the heavy chains of the anti-CD47 antibody.

The bispecific antibodies can also be asymmetrical, such as those illustrated in FIG. 3E-3F. In FIG. 3E, two single domain anti-PD-L1 antibodies are connected, in series, to the N-terminus of one of the Fc chains. On the other Fc chain, an anti-CD47 Fab unit is fused to the N-terminus. Slightly differently, in FIG. 3F, the anti-CD47 portion includes a single chain fragment (scFv).

The bispecific antibodies may include constant regions from any IgG types, such as IgG1 and IgG4.

Also provided are compositions that include the antibody or the polypeptide and a pharmaceutically acceptable carrier.

It will also be understood by one of ordinary skill in the art that antibodies as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, 98%, or 99% identical to the starting sequence. In some embodiments, the modified antibody or fragment retains the designate CDR sequences.

The present disclosure provides bi- and multi-specific antibodies that have binding specificities at least to the human PD-L1 and TIGIT proteins. PD-L1 is a critical “don't find me” signal to the adaptive immune system, whereas TIGIT helps tumor and infect cells to evade from immune responses. They are often overexpressed on human tumors. Thus, dual targeting both innate and adaptive immune checkpoints would likely maximize anti-tumor therapeutic effect and elicit more durable responses.

In some embodiments, the bi- and multi-specific antibodies include an anti-PD-L1 portion which includes at least a single domain anti-PD-L1 antibody. As demonstrated, the single chain anti-PD-L1 antibodies have high affinity to the human PD-L1 protein. The antibodies exhibited potent binding and inhibitory activities and are useful for therapeutic and diagnostics uses.

In some embodiments, wherein the single domain antibody has binding specificity to the human PD-L1 protein and comprises a complementarity determining region 1 (CDR1), a CDR2 and a CDR3. In some embodiments, the CDR1, CDR2 and CDR3 include the amino acid sequences of SEQ ID NO: 55, 56 and 57, respectively. In some embodiments, the CDR1, CDR2 and CDR3 include the amino acid sequences of SEQ ID NO: 113, 49 and 50, respectively. In some embodiments, the CDR1, CDR2 and CDR3 include CDR1, CDR2 and CDR3, respectively, of any one of the antibodies provided in Table 1 (e.g., SEQ ID NO: 1-36).

In one embodiment, in the antibody, the CDR1 includes the amino acid sequence of SEQ ID NO: 55, the CDR2 includes the amino acid sequence of SEQ ID NO: 56, and the CDR3 includes the amino acid sequence of SEQ ID NO: 57. SEQ ID NO: 55, 56 and 57 are the CDRs of antibody ALP-Tan-3p-93, and its humanized counterparts 93_VH-1 through 93_VH-9. In some embodiments, the CDR1, CDR2 and CDR3 include SEQ ID NO: 55, 56 and 57 but with one, two, or three amino acid additions, deletions, and/or substitutions, respectively. In some embodiments, the substitutions are conservative substitutions.

In some embodiments, the antibody is humanized. In some embodiments, the humanized antibody includes one or more back mutations tested to improve the properties of the grafted antibody. In some embodiments, the back mutations are selected from the group consisting of 37F, 47F, 49A, 78V and 94A, according to Kabat numbering. In some embodiments, the humanized antibody includes all of back mutations of 37F, 47F, 49A, 78V and 94A, according to Kabat numbering.

Example humanized antibodies include 93_VH-1, 93_VH-2, 93_VH-3, 93_VH-4, 93_VH-5, 93_VH-6, 93_VH-7, 93_VH-8, and 93_VH-9. Example sequences are SEQ ID NO: 114-122. In some embodiments, the humanized antibody has the amino acid sequence of SEQ ID NO: 119.

In some embodiments, in the antibody, the CDR1 includes the amino acid sequence of SEQ ID NO: 113, the CDR2 includes the amino acid sequence of SEQ ID NO: 49, and the CDR3 includes the amino acid sequence of SEQ ID NO: 50. SEQ ID NO: 113, 49 and 50 are the CDRs of humanized antibodies 112-VHH1-PTM, 112-VHH2-PTM, 112-VHH3-PTM, 112-VHH4-PTM, 112-VHH5-PTM, 112-VHH6-PTM, or 112-VHH7-PTM. Compared to the original antibody ALP-Tan-3p-112, these humanized antibodies included a N34Q substitution (Kabat numbering) in CDR1 (SEQ ID NO: 48), to prevent posttranslational modification. In some embodiments, the CDR1, CDR2 and CDR3 include SEQ ID NO: 113, 49 and 50 but with one, two, or three amino acid additions, deletions, and/or substitutions, respectively. In some embodiments, the substitutions are conservative substitutions.

In some embodiments, the antibody is humanized. In some embodiments, the humanized antibody includes one or more back mutations selected from the group consisting of 37Y, 44Q, 45R, 49A, 68A, 93R, and 94V, according to Kabat numbering. In some embodiments, the humanized antibody includes all of back mutations of 37Y, 44Q, 45R, 49A, 68A, 93R, and 94V, according to Kabat numbering. In some embodiments, the antibody comprises an amino acid sequence selected from the group consisting of SEQ ID NO: 123-130. In some embodiments, the antibody comprises the amino acid sequence of SEQ ID NO: 127. In some embodiments, the antibody comprises the amino acid sequence of SEQ ID NO: 130.

In some embodiments, in the antibody, the CDR1, CDR2 and CDR3 include CDR1, CDR2 and CDR3, respectively, of any one of the antibodies provided in Table 1 (e.g., SEQ ID NO: 1-36). In one embodiment, the CDR1, CDR2 and CDR3 include the amino acid sequences of SEQ ID NO: 37, 38, and 39, respectively. In one embodiment, the CDR1, CDR2 and CDR3 include the amino acid sequences of SEQ ID NO: 40, 41, and 42, respectively. In one embodiment, the CDR1, CDR2 and CDR3 include the amino acid sequences of SEQ ID NO: 43, 44, and 45, respectively. In one embodiment, the CDR1, CDR2 and CDR3 include the amino acid sequences of SEQ ID NO: 40, 46, and 47, respectively.

In one embodiment, the CDR1, CDR2 and CDR3 include the amino acid sequences of SEQ ID NO: 40, 46, and 47, respectively. In one embodiment, the CDR1, CDR2 and CDR3 include the amino acid sequences of SEQ ID NO: 48, 49, and 50, respectively. In one embodiment, the CDR1, CDR2 and CDR3 include the amino acid sequences of SEQ ID NO: 40, 51, and 52, respectively. In one embodiment, the CDR1, CDR2 and CDR3 include the amino acid sequences of SEQ ID NO: 37, 53, and 54, respectively. In one embodiment, the CDR1, CDR2 and CDR3 include the amino acid sequences of SEQ ID NO: 40, 41, and 47, respectively.

In one embodiment, the CDR1, CDR2 and CDR3 include the amino acid sequences of SEQ ID NO: 40, 46, and 47, respectively. In one embodiment, the CDR1, CDR2 and CDR3 include the amino acid sequences of SEQ ID NO: 55, 56, and 57, respectively. In one embodiment, the CDR1, CDR2 and CDR3 include the amino acid sequences of SEQ ID NO: 40, 41, and 58, respectively. In one embodiment, the CDR1, CDR2 and CDR3 include the amino acid sequences of SEQ ID NO: 37, 53, and 39, respectively. In one embodiment, the CDR1, CDR2 and CDR3 include the amino acid sequences of SEQ ID NO: 37, 59, and 60, respectively.

In one embodiment, the CDR1, CDR2 and CDR3 include the amino acid sequences of SEQ ID NO: 61, 62, and 63, respectively. In one embodiment, the CDR1, CDR2 and CDR3 include the amino acid sequences of SEQ ID NO: 64, 65, and 66, respectively. In one embodiment, the CDR1, CDR2 and CDR3 include the amino acid sequences of SEQ ID NO: 67, 68, and 69, respectively. In one embodiment, the CDR1, CDR2 and CDR3 include the amino acid sequences of SEQ ID NO: 70, 71, and 72, respectively. In one embodiment, the CDR1, CDR2 and CDR3 include the amino acid sequences of SEQ ID NO: 37, 73, and 74, respectively.

In one embodiment, the CDR1, CDR2 and CDR3 include the amino acid sequences of SEQ ID NO: 37, 75, and 76, respectively. In one embodiment, the CDR1, CDR2 and CDR3 include the amino acid sequences of SEQ ID NO: 64, 77, and 66, respectively. In one embodiment, the CDR1, CDR2 and CDR3 include the amino acid sequences of SEQ ID NO: 37, 78, and 79, respectively. In one embodiment, the CDR1, CDR2 and CDR3 include the amino acid sequences of SEQ ID NO: 80, 81, and 82, respectively. In one embodiment, the CDR1, CDR2 and CDR3 include the amino acid sequences of SEQ ID NO: 40, 83, and 47, respectively.

In one embodiment, the CDR1, CDR2 and CDR3 include the amino acid sequences of SEQ ID NO: 84, 85, and 86, respectively. In one embodiment, the CDR1, CDR2 and CDR3 include the amino acid sequences of SEQ ID NO: 64, 87, and 66, respectively. In one embodiment, the CDR1, CDR2 and CDR3 include the amino acid sequences of SEQ ID NO: 84, 88, and 89, respectively. In one embodiment, the CDR1, CDR2 and CDR3 include the amino acid sequences of SEQ ID NO: 84, 90, and 91, respectively. In one embodiment, the CDR1, CDR2 and CDR3 include the amino acid sequences of SEQ ID NO: 92, 93, and 94, respectively. In one embodiment, the CDR1, CDR2 and CDR3 include the amino acid sequences of SEQ ID NO: 95, 96, and 97, respectively. In one embodiment, the CDR1, CDR2 and CDR3 include the amino acid sequences of SEQ ID NO: 84, 98, and 99, respectively.

In one embodiment, the CDR1, CDR2 and CDR3 include the amino acid sequences of SEQ ID NO: 100, 101, and 102, respectively. In one embodiment, the CDR1, CDR2 and CDR3 include the amino acid sequences of SEQ ID NO: 103, 104, and 105, respectively. In one embodiment, the CDR1, CDR2 and CDR3 include the amino acid sequences of SEQ ID NO: 84, 85, and 106, respectively. In one embodiment, the CDR1, CDR2 and CDR3 include the amino acid sequences of SEQ ID NO: 107, 108, and 109, respectively. In one embodiment, the CDR1, CDR2 and CDR3 include the amino acid sequences of SEQ ID NO: 110, 111, and 112, respectively.

In some embodiments, the antibody includes an amino acid sequence selected from SEQ ID NO: 1-36.

Also provided, in some embodiments, are anti-PD-L1 antibodies and antigen binding fragments that compete with any of the antibodies disclosed herein in binding to human PD-L1. Also provided, in some embodiments, are anti-PD-L1 antibodies and antigen binding fragments that bind to the same epitope as any of the antibodies disclosed herein. Also provided, in some embodiments, are anti-PD-L1 antibodies and antigen binding fragments that included the VH CDR1, CDR2, and CDR3 and VL CDR1, CDR2 and CDR3 of the antibodies disclosed herein.

In some embodiments, the anti-TIGIT portion of the bi- or multi-specific antibodies has a pair (or, in some embodiments, two pairs) of heavy chain variable region (VH) and a light chain variable region (VL). The VH can include a VH CDR1, a VH CDR and a VH CDR3. The VL can include a VL CDR1, a VL CDR2 and a VL CDR3.

In some embodiments, the VH CDR1, VH CDR, VH CDR3, VL CDR1, VL CDR2 and VL CDR3 include the amino acid sequences of SEQ ID NO: 173-178, respectively. In some embodiments, the VH includes the amino acid sequence of SEQ ID NO: 171, and the VL includes the amino acid sequence of SEQ ID NO: 172 (Table 7).

The bispecific antibody can take any format, including those illustrated in FIG. 9. In one embodiment, the bispecific antibody is preferably symmetrical. In one embodiment, the single domain anti-PD-L1 antibodies are located at the C-terminal side of the anti-TIGIT portions.

An example format is provided in FIG. 9A, in which two single domain anti-PD-L1 antibodies are fused, optionally through a linker, to the C-terminus of each of the heavy chain constant regions of the anti-TIGIT antibody. In the example of FIG. 9B, each heavy chain includes two copies of the single domain anti-PD-L1 antibodies.

In the example of FIG. 9C, the single domain anti-PD-L1 antibodies are fused, optionally through a linker, to the C-terminus of each of the light chains (constant regions) of the anti-TIGIT antibody.

The bispecific antibodies may include constant regions from any IgG types, such as IgG1 and IgG4.

Also provided are compositions that include the antibody or the polypeptide and a pharmaceutically acceptable carrier.

It will also be understood by one of ordinary skill in the art that antibodies as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, 98%, or 99% identical to the starting sequence. In some embodiments, the modified antibody or fragment retains the designate CDR sequences.

### Polynucleotides Encoding the Antibodies and Methods of Preparing the Antibodies

The present disclosure also provides isolated polynucleotides or nucleic acid molecules encoding the antibodies, variants or derivatives thereof of the disclosure. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

Methods of making antibodies are well known in the art and described herein. In certain embodiments, both the variable and constant regions of the antigen-binding polypeptides of the present disclosure are fully human. Fully human antibodies can be made using techniques described in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140 which are incorporated by reference in their entireties.

### Cancer Treatment

As described herein, the antibodies, bispecific antibodies, polypeptides, variants or derivatives of the present disclosure may be used in certain treatment and diagnostic methods.

The present disclosure is further directed to antibody-based therapies which involve administering the antibodies of the disclosure to a patient such as an animal, a mammal, and a human for treating one or more of the disorders or conditions described herein. Therapeutic compounds of the disclosure include, but are not limited to, antibodies of the disclosure (including variants and derivatives thereof as described herein) and nucleic acids or polynucleotides encoding antibodies of the disclosure (including variants and derivatives thereof as described herein).

The antibodies of the disclosure can also be used to treat or inhibit cancer. PD-L1 can be overexpressed in tumor cells. Tumor-derived PD-L1 can bind to PD-1 on immune cells thereby limiting antitumor T-cell immunity. Results with small molecule inhibitors, or monoclonal antibodies targeting PD-L1 in murine tumor models, indicate that targeted PD-L1 therapy is an important alternative and realistic approach to effective control of tumor growth. As demonstrated in the experimental examples, the anti-PD-L1 antibodies activated the adaptive immune response machinery, which can lead to improved survival in cancer patients.

Accordingly, in some embodiments, provided are methods for treating a cancer in a patient in need thereof. The method, in one embodiment, entails administering to the patient an effective amount of an antibody of the present disclosure. In some embodiments, at least one of the cancer cells (e.g., stromal cells) in the patient expresses, over-express, or is induced to express PD-L1. Induction of PD-L1 expression, for instance, can be done by administration of a tumor vaccine or radiotherapy.

Tumors that express the PD-L1 protein include those of bladder cancer, non-small cell lung cancer, renal cancer, breast cancer, urethral cancer, colorectal cancer, head and neck cancer, squamous cell cancer, Merkel cell carcinoma, gastrointestinal cancer, stomach cancer, oesophageal cancer, ovarian cancer, renal cancer, and small cell lung cancer. Accordingly, the presently disclosed antibodies can be used for treating any one or more such cancers.

### Compositions

The present disclosure also provides pharmaceutical compositions. Such compositions comprise an effective amount of an antibody, and an acceptable carrier. In some embodiments, the composition further includes a second anticancer agent (e.g., an immune checkpoint inhibitor).

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### EXAMPLES

### Example 1. Generation of Alpaca Single Domain Antibodies Against Human PD-L1

This example shows how anti-human-PD-L1 single domain antibodies were generated using immunization of alpaca followed by phage library construction and selection.

Antigen: Recombinant human PD-L1/hFc fusion proteins were used as the immunogen to raise anti-human PD-L1 antibodies. A fusion protein comprising the entire extracellular region of human PD-L1 fused to a human immunoglobulin Fc domain was used as the immunogen.

### Immunization

Alpacas were first subcutaneously (SC) immunized with a 1:1 mixture of 600 μg mouse PD-L1 and complete Freud's adjuvant on day 0 and immunized with 250 μg mouse PD-L1 with incomplete Freud's adjuvant on day 21 and 250 g human PD-L1 with incomplete Freud's adjuvant on day 42. The immune response was monitored by measuring titers for anti-PD-L1 binding.

### Library Construction and Screening

Alpaca PBMCs were collected, and an antibody phage display library was generated by RNA isolation, cDNA reverse transcription, PCR amplification and cloning into a phage display vector. The library was then subjected for one round of liquid phase panning and one round of solid phase panning. In general, the libraries were incubated in biotinylated PD-L1-coated immunotubes or beads. Unbound phages were removed by washing with PBST for 5-20 times. For each selection, three rounds of panning were performed in total.

The binder sequences were amplified from antigen-binding positive phages by PCR and confirmed by DNA sequencing. Sequences of the unique antibodies and their CDR regions are provided in the table below.

TABLE 1
Antibody Sequences

| Antibody | Sequence | SEQ ID NO: |
|---|---|---|
| ALP-Tan- | EVQLVESGGGLVQAGDSLTLSCAASGRTFSSYAMGWFRQAPGKEREFVARITWTGRST | 1 |
| 3p-100 | SYADSVKGRFTISRDNAKNRVYLRMNSLKPEDTAVYYCAADLEGAMVSRRREIEYGHW | |
| | GQGTQVTVSS | |
| ALP-Tan- | EVDLVESGGGLVQAGGSLRLSCAASGGSTFAMAWLRQAPGKEREFVAAVGRSPRSPGI | 2 |
| 3p-101 | TYYADSVKGRFTISRDNAKNTVYLQMNSLKPEDTAVYYCAAGGILGPRAHYDYWGQGT | |
| | QVTVSS | |
| ALP-Tan- | QVQLVESGGGLVQAGGSLRLSCAASGRTFSRYAMGWFRQAPGKEREFVAAISWSGGTT | 3 |
| 3p-102 | NYADSVKGRFTISRDNAKITVYLQMNSLKPEDTAFYYCAAGKRLTLRSSGYKYWGQGT | |
| | QVTVSS | |
| ALP-Tan- | AVQLVESGGGLVQAGGSLRLSCAASRGSTFAMAWIRQAPGKEREFVAAVGRSPRGPGI | 4 |
| 3p-104 | TYYADSVKGRFTISRDNANNTVYLQMNSLKPEDTAVYYCAAGGILGPRAQYDYWGQGT | |
| | QVTVSS | |
| ALP-Tan- | QVQLVESGGGLVQAGSSLRLSCAASRGSTFAMAWIRQAPGKEREFVAAVGRSPRGPGI | 5 |
| 3p-106 | TYYADSVKGRFTISRDNANNTVYLQMNSLKPEDTAVYYCAAGGILGPRAQYDYWGQGT | |
| | QVTVSS | |
| ALP-Tan- | QVQLVESGGGLVQPGGSLRLSCAASGSIFSSGTNFSDSKIDWYRQAPGKQRDWIAGIF | 6 |
| 3p-112 | STGSTIYEDSVKGRFAISRDNAKNMGYLQMNSLKPEDTAVYYCRVIGRGILAWGQGTQ | |
| | VTVSS | |
| ALP-Tan- | QVQLVESGGGLVQAGGSLRLSCAASRGSTFAMAWIRQAPRKEREFVPAVGRSPLGPVI | 7 |
| 3p-118 | TYYADSVKGRFTISRDNANNTVYLQMNSLKPEDTAVDYCASCGILGPRAHYDYWGQGT | |
| | HVTVSS | |
| ALP-Tan- | EVQLVESGGGLVQAGDSLTLSCAASGRTFSSYAMGWFRQAPGKEREFVARITWSGRST | 8 |
| 3p-124 | SYADSVKGRFTISRDNAKNRVYLRMNSLKPEDTAVYYCAADLEGAMVSRRREIEYGQW | |
| | GQGTQVTVSS | |
| ALP-Tan- | QVQLVESGGGLVQAGGSLRLSCAASGGSTFAMAWIRQAPGKEREFVAAVGRSPRSPGI | 9 |
| 3p-127 | TYYADSVKGRFTISRDNANNTVYLQMNSLKPEDTAVYYCAAGGILGPRAQYDYWGQGT | |
| | QVTVSS | |
| ALP-Tan- | QVQLVESGGGLVQAGSSLRLSCAASRGSTFAMAWIRQAPGKEREFVAAVGRSPRGPGI | 10 |
| 3p-89 | TYYADSVKGRFTISRDNANNTVYLQMNSLKPEDTAVYYCAAGGILGPRAQYDYWGQGT | |
| | QVTVSS | |
| ALP-Tan- | QVQLVESVGGLVQPGDSLRLSCLASGRTFTFRHYVMGWFRQAPGKEREFVAAISWSGS | 11 |
| 3p-93 | GSYYADSVKGRFTISRDNSKNMVFLQMNGLKPEDTAVYYCAADMTTRMSQASREYDYW | |
| | GQGTQVTVSS | |
| ALP-Tan- | EVQLVESGGGLVQAGGSLRLSCAASGGSTFAMAWLRQAPGKEREFVAAVGRSPRSPGI | 12 |
| 3p-95 | TYYADSVKGRFTISRDNAKNTVWLQMNSLKPEDTAVYYCAAGGILGPRAEYDYWGQGT | |
| | RVTVSS | |
| ALP-Tan- | QVHLVESGGGLVQAGDSLTLSCAASGRTFSSYAMGWFRQAPGKEREFVARITWSGRST | 13 |
| 3p-99 | SYADSVKGRFTISRDNAKNRVYLRMNSLKPEDTAVYYCAADLEGAMVSRRREIEYGHW | |
| | GQGTQVTVSS | |
| ASP-1P-1 | QVQLVESGGELVQAGGSLRLSCAASGRTFSSYAMGWFRQGPGKEREFVAAISASGGRT | 14 |
| | YYADSVKGRFTISRDNAKNTVYLQMNSLKPEDTAVYYCAVAGPRIRIATITLSREYDY | |
| | WGQGTQVTVSS | |
| ASP-3P-10 | AVQLVESGGGLVQAGGSLRLSCVASEIAFSVFDMGWYRQAPGKQRELAASIGHDGRIN | 15 |
| | YADSVKGRFTISRDNAKNTVHLQMNTLKSEDTAVYYCNARNSFRDLWGQGTQVTVSS | |
| ASP-3P-13 | AVQLVESGGGLVQPGGSLRLSCAASGRSFSGYAMGWFRQAPGKEREFVSAISGSGRNT | 16 |
| | YYADSVKGRFTISRDNAKNTMYLQMNSLKPEDTAVYYCAVAGPAITIATMTLRGKYDY | |
| | WGQGTQVTVSS | |
| ASP-1P-2 | QVHLVESGGGLVQAGDSLRLSCAASGRTFSSRAMGWFRQAPGKEREFVAAISASGSRT | 17 |
| | YYADSVKGRFTISRDNAKNTVYLQMNSLKPEDTAVYYCAAAGPRITIATMTLSREYDY | |
| | WGQGTQVTVSS | |
| ASP-1P-3 | EVQLVESGGGLVQPGGSLRLSCAASGRTFSSYALGWFRQAPGKEREFVAAISASGLRT | 18 |
| | YYADSVKGRFTISRDNAKNTVYLQMNSLKPEDTAVYYCAEAGPRIRIAIMTLSREYDY | |
| | WGQGTLVTVSS | |
| ASP-2P-4 | QVQLVESGGGLVQAGGSLRLSCAASGRTFSSYAMGWFRQAPGKEREFATAISASGRST | 19 |
| | YYADSVKGRFTISRDNAKNTVYLQMNSLKPEDTAVYYCAQGGPSITIRIMGSSSKYDY | |
| | WGRGTQVTVSS | |
| ASP-2P-8 | QVHLVESGGGLVQAGGSLRLSCAASGRTFSSYAMGWFRQAPGKEREFVAAVSASGGRS | 20 |
| | YYVDSVKGRFTISRDNAKNTVYLQMNSLKPEDTAVYYCAAAGRSITIATMTERYKYDY | |
| | WGQGTQVTVSS | |
| ASP-2P-14 | EVQLVESGGGLVQAGGSLRLSCAASGRSFSGYAMGWFRQAPGKERDFVAAISGTGGST | 21 |
| | YYVDSVKGRFTISRDNAKNTMYLQMNSLKPEDTAVYYCAVAGPAITIATMTLRGKYDY | |
| | WGQGTQVTVSS | |
| ASP-2P-15 | AVQLVESGGSLRLSCAASGRTFSSYAMGWFRQAPGKEREFVAAISGSGARTYYADSVK | 22 |
| | GRFTISRANTKNTVYLQMNSLKPEDTAVYYCAADATRIASVDVPKSWGYWGQGTQVTV | |
| | SS | |
| ASP-2P-17 | QLHFVESGGGLVQAGGSLRLACAASGRTFSGYARTWFRQAPGKEREFVAAISGSGASA | 23 |
| | YYADSVKGRFTISRDNAKNTVYLQMNSLKPEDTAVYYCAADQSIRIATMRTHAAYGYW | |
| | GQGTQVTVSS | |
| ASP-2P-18 | QVQLVESVGGLVQAGGSLSLSCAASGGSTFAMAWLRQAPGKEREFVAAVGRSPRGPGI | 24 |
| | TNYADSVKGRFTISRDNAKNTVYLQMNSLKPEDTAVYYCAAGGILGPRAQYDYWGQGT | |
| | RVTVSS | |
| ASP-3P-26 | EVHLVESGGGLVQPGGSLRLSCAHSGSIRSINVMNWYRQVPGKQRELVATITAGGSIN | 25 |
| | YADSVKGRFTISRDNALNTAALQMNSLRPEDTAVYYCHADKILTYNGVIYRAEYDVWG | |
| | QGTQVTVSS | |
| ASP-3P-27 | QVQLVESGGGLVQAGGSLRLSCAASGRTFSGYAMGWFRQAPGKEREFVAAISGSGGRT | 26 |
| | YYVDSAKGRFTISRDNAKNTMYLQMNSLKPEDTAVYYCAVAGPAITIATMTLRGKYDY | |
| | WGQGTQVTVSS | |
| ASP-3P-29 | QVQLVESGGGLVQPGGSLRLSCAASESIRSINVMNWYRQAPGKQRELVATITSGGTTT | 27 |
| | YADSVKGRFTSSRDNAKNTVALQMNSLRPEDTAVYYCHADKVLYYNGVIYGAEYDVWG | |
| | QGTQVTVSS | |
| ASP-3P-30 | QLQLVESGGGLVQPGGSLRLSCAPSGSIRSINVMNWYRQAPGKQRDLVATITSGGSIN | 28 |
| | YADSVKGRFIISRDNAKNTVALQMNSLRPEDTAVYYCHADKVLYYNGVLYGAEYDVWG | |
| | QGTQVTVSS | |
| ASP-3P-35 | EVHLVESGGGLVQAGGSLRLSCEVSGSIFSGTHFSFNTMGWYRQAPGKQRELVALGRG | 29 |
| | SRGINYADSVKGRFTFSSDNAKNTIFLQMNNLEPEDTGNYTCYVRRPSGSYAGQYYPD | |
| | SSEYWGQGTQVTVSS | |
| ASP-3P-36 | QPQVVESGGGLVQAGGSLRLSCVASGSNFAFEYIAWYRQAPGKEREVVALISPQSITT | 30 |
| | YADSVKGRFTISRDNAKSTVYLQMNSLKPEDTAVYYCHDREYWGQGTQVTVSS | |
| ASP-3P-38 | QLHFVESGGGLVQPGGSLRLSCAASGSIRSINVMNYYRQAPGKQRELVATISSVGSIN | 31 |
| | YADSVKGRFTISRDNAQNTVALQMNSLRPEDTAVYYCHADKVLYYNGVMYGVESDVWG | |
| | QGTQVTVSS | |
| ASP-3P-40 | QVQLVESGGGEVQPGGSLRLSCAASGPTFSRYIMGWFRQAPGKEREFVAAISRIGGIT | 32 |
| | YYTDSVKGRFTISRDNAKNTVYLQMNSLEPEDTASYYCAAKSSSSSSKYTARGADAYD | |
| | YWGQGTQVTVSS | |
| ASP-3P-43 | AVQLVESGGGLVQPGGSLRLSCAASRSVFSVLVMGWYRQAPGQQRELVATISNEGYSN | 33 |
| | YADSVKGRFAISRDNAKKTVYLQMNSLKPEDTAVYYCNAAWGNGRYTYWGQGTQVTVS | |
| | S | |
| ASP-3P-44 | EVHLVESGGGLVQPGGSLRLSCAHSGSIRSINVMNWYRQVPGKQRELVATITAGGSIN | 34 |
| | YADSVKGRFTISRDNAKNSAALQMNSLRPEDTAVYYCHADKVLSYNGVIYRAEYDVWG | |
| | QGTQVTVSS | |
| ASP-3P-45 | ELQLVESGGGLVQAGGSLRLSCAVSESIFKFPRMGWYRQGPGDQRDLVALSRSSGSTE | 35 |
| | YADFAKGRFTISRDNSKNTVYLQMNSLKPEDSGTYYCYVRRPSGSENGRWYIDPSDDW | |
| | GQGTRVTVSS | |
| ASP-3P-46 | QVQLVESGGGLVQPGGSLRLSCAASARSINGMEWYRQAPGERRELVAGITAGGSAYYT | 36 |
| | DTVKGRFTISRDNAENTGYLQMNSLSPDDTAVYYCRRQYGPNWYWGQGTQVTVSS | |

TABLE 1A
CDR sequences

| No. | Sequence | SEQ ID NO: |
|---|---|---|
| ALP-Tan-3p- | SYAMG | 37 |
| 100 | RITWTGRSTSYADSVKG | 38 |
| | DLEGAMVSRRREIEYGH | 39 |
| ALP-Tan-3p- | AMA | 40 |
| 101 | AVGRSPRSPGITYYADSVKG | 41 |
| | GGILGPRAHYDY | 42 |
| ALP-Tan-3p- | RYAMG | 43 |
| 102 | AISWSGGTTNYADSVKG | 44 |
| | GKRLTLRSSGYKY | 45 |
| ALP-Tan-3p- | AMA | 40 |
| 104 | AVGRSPRGPGITYYADSVKG | 46 |
| | GGILGPRAQYDY | 47 |
| ALP-Tan-3p- | AMA | 40 |
| 106 | AVGRSPRGPGITYYADSVKG | 46 |
| | GGILGPRAQYDY | 47 |
| ALP-Tan-3p- | SGTNFSDSKID | 48 |
| 112 | GIFSTGSTIYEDSVKG | 49 |
| | IGRGILA | 50 |
| ALP-Tan-3p- | AMA | 40 |
| 118 | AVGRSPLGPVITYYADSVKG | 51 |
| | CGILGPRAHYDY | 52 |
| ALP-Tan-3p- | SYAMG | 37 |
| 124 | RITWSGRSTSYADSVKG | 53 |
| | DLEGAMVSRRREIEYGQ | 54 |
| ALP-Tan-3p- | AMA | 40 |
| 127 | AVGRSPRSPGITYYADSVKG | 41 |
| | GGILGPRAQYDY | 47 |
| ALP-Tan-3p-89 | AMA | 40 |
| | AVGRSPRGPGITYYADSVKG | 46 |
| | GGILGPRAQYDY | 47 |
| ALP-Tan-3p-93 | RHYVMG | 55 |
| | AISWSGSGSYYADSVKG | 56 |
| | DMTTRMSQASREYDY | 57 |
| ALP-Tan-3p-95 | AMA | 40 |
| | AVGRSPRSPGITYYADSVKG | 41 |
| | GGILGPRAEYDY | 58 |
| ALP-Tan-3p-99 | SYAMG | 37 |
| | RITWSGRSTSYADSVKG | 53 |
| | DLEGAMVSRRREIEYGH | 39 |
| ASP-1P-1 | SYAMG | 37 |
| | AISASGGRTYYADSVKG | 59 |
| | AGPRIRIATITLSREYDY | 60 |
| ASP-3P-10 | VFDMG | 61 |
| | SIGHDGRINYADSVKG | 62 |
| | RNSFRDL | 63 |
| ASP-3P-13 | GYAMG | 64 |
| | AISGSGRNTYYADSVKG | 65 |
| | AGPAITIATMTLRGKYDY | 66 |
| ASP-1P-2 | SRAMG | 67 |
| | AISASGSRTYYADSVKG | 68 |
| | AGPRITIATMTLSREYDY | 69 |
| ASP-1P-3 | SYALG | 70 |
| | AISASGLRTYYADSVKG | 71 |
| | AGPRIRIATMTLSREYDY | 72 |
| ASP-2P-4 | SYAMG | 37 |
| | AISASGRSTYYADSVKG | 73 |
| | GGPSITIRTMGSSSKYDY | 74 |
| ASP-2P-8 | SYAMG | 37 |
| | AVSASGGRSYYVDSVKG | 75 |
| | AGRSITIATMTERYKYDY | 76 |
| ASP-2P-14 | GYAMG | 64 |
| | AISGTGGSTYYVDSVKG | 77 |
| | AGPAITIATMTLRGKYDY | 66 |
| ASP-2P-15 | SYAMG | 37 |
| | AISGSGARTYYADSVKG | 78 |
| | DATRIASVDVPKSWGY | 79 |
| ASP-2P-17 | GYART | 80 |
| | AISGSGASAYYADSVKG | 81 |
| | DQSIRIATMRTHAAYGY | 82 |
| ASP-2P-18 | AMA | 40 |
| | AVGRSPRGPGITNYADSVKG | 83 |
| | GGILGPRAQYDY | 47 |
| ASP-3P-26 | INVMN | 84 |
| | TITAGGSTNYADSVKG | 85 |
| | DKILTYNGVIYRAEYDV | 86 |
| ASP-3P-27 | GYAMG | 64 |
| | AISGSGGRTYYVDSAKG | 87 |
| | AGPAITIATMTLRGKYDY | 66 |
| ASP-3P-29 | INVMN | 84 |
| | TITSGGTTTYADSVKG | 88 |
| | DKVLYYNGVIYGAEYDV | 89 |
| ASP-3P-30 | INVMN | 84 |
| | TITSGGSTNYADSVKG | 90 |
| | DKVLYYNGVLYGAEYDV | 91 |
| ASP-3P-35 | GTHFSFNTMG | 92 |
| | LGRGSRGINYADSVKG | 93 |
| | RRPSGSYAGQYYPDSSEY | 94 |
| ASP-3P-36 | FEYIA | 95 |
| | LISPQSITTYADSVKG | 96 |
| | REY | 97 |
| ASP-3P-38 | INVMN | 84 |
| | TISSVGSTNYADSVKG | 98 |
| | DKVLYYNGVMYGVESDV | 99 |
| ASP-3P-40 | RYIMG | 100 |
| | AISRIGGITYYTDSVKG | 101 |
| | KSSSSSSKYTARGADAYDY | 102 |
| ASP-3P-43 | VLVMG | 103 |
| | TISNEGYSNYADSVKG | 104 |
| | AWGNGRYTY | 105 |
| ASP-3P-44 | INVMN | 84 |
| | TITAGGSTNYADSVKG | 85 |
| | DKVLSYNGVIYRAEYDV | 106 |
| ASP-3P-45 | FPRMG | 107 |
| | LSRSSGSTEYADFAKG | 108 |
| | RRPSGSFNGRWYTDPSDD | 109 |
| ASP-3P-46 | GME | 110 |
| | GITAGGSAYYTDTVKG | 111 |
| | QYGPNWY | 112 |

### Example 2. Binding and Blocking Activity of Alpaca Monoclonal Antibodies Against Human PD-L1

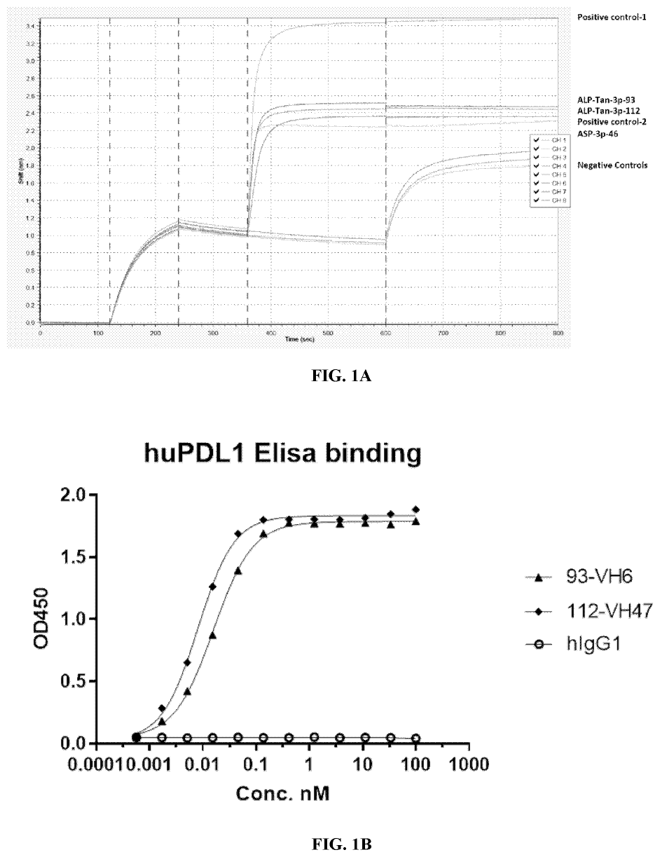

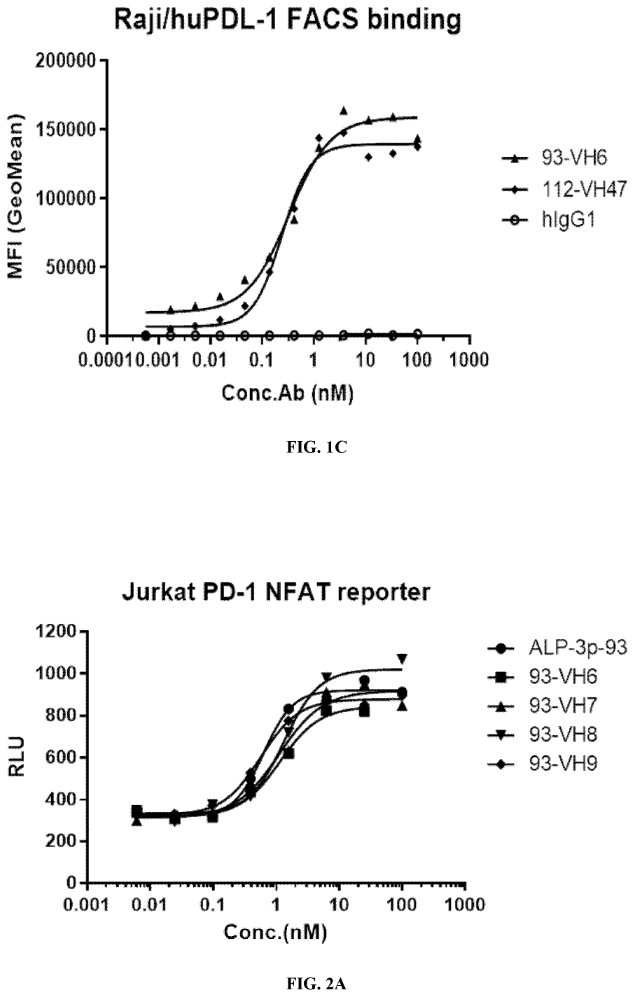

The binding and blocking property of some of the antibodies were characterized by Gator. Anti-his probe was first loaded onto the chip and followed by human PD-L1-his to capture the antigen. Then, the antibodies were injected to record the binding curve. Finally, human PD1/hFc was injected to determine whether the antibodies could block the interaction between PD-1 and PD-L1. As shown in the FIG. 1, all of ALP-Tan-3p-112, ALP-Tan-3p-93 and ASP-30-46 effectively blocked the interaction between PD-1 and PD-L1. The affinity was further confirmed by Biacore T200.

TABLE 2
Affinities

| | Kon (1/Ms) | Koff (1/s) | KD (M) |
|---|---|---|---|
| ALP-Tan-3p-112 | 4.9 × 10⁵ | 0.000235 | 4.79 × 10⁻¹⁰ |
| ALP-Tan-3p-93 | 1.54 × 10⁵ | 7.8 × 10⁻⁵ | 4.59 × 10⁻¹⁰ |
| ASP-3p-46 | 7.1 × 10⁵ | 0.00128 | 1.81 × 10⁻⁹ |

### Example 3. Humanization of Anti-PD-L1 Alpaca Monoclonal Antibodies

The mAb ALP-Tan-3p-93 and ALP-Tan-3p-112 variable region genes were employed to create a humanized mAb. In the first step of this process, the amino acid sequences of the ALP-Tan-3p-93 and ALP-Tan-3p-112 were compared against the available database of human Ig gene sequences to find the overall best-matching human germline Ig gene sequences. For ALP-Tan-3p-93, the closest human match was IGHV3H23*04 gene. Humanized variable domain sequences were then designed where the CDR1, 2 and 3 of the ALP-Tan-3p-93 were grafted onto framework sequences of the IGHV3-23*04 gene. For ALP-Tan-3p-112, the closest human match was IGHV3-48*03 gene. Humanized variable domain sequences were then designed where the CDR1, 2 and 3 of the ALP-Tan-3p-112 were grafted onto framework sequences of the IGHV3-48*03 gene. Meanwhile, one residue mutation (N34Q, Kabat numbering) was introduced into CDR1 to reduce the risk of posttranslational modification. A 3D model was then generated to determine if there were any framework positions where replacing the alpaca amino acid to the human amino acid could affect binding and/or CDR conformation.

TABLE 3
Humanized antibodies and back mutations

| Chain | Sequence | SEQ ID NO: |
|---|---|---|
| 93 VH-1 | EVQLVESGGGLVQPGGSLRLSCAASGFTFTFRHYVMGWVRQAPGKEREWVSAISWSG | 114 |
| | SGSYYADSVKGRFTISRDNSKNTLYLQMNSLRAEDTAVYYCAKDMTTRMSQASREYD | |
| | YWGQGTLVTVSS | |
| 93_VH-2 | EVQLVESGGGLVQPGGSLRLSCAASGFTFTFRHYVMGWVRQAPGKEREWVSAISWSG | 115 |
| | SGSYYADSVKGRFTISRDNSKNTVYLQMNSLRAEDTAVYYCAADMTTRMSQASREYD | |
| | YWGQGTLVTVSS | |
| 93 VH-3 | EVQLVESGGGLVQPGGSLRLSCAASGFTFTFRHYVMGWFRQAPGKGLEFVAAISWSG | 116 |
| | SGSYYADSVKGRFTISRDNSKNTLYLQMNSLRAEDTAVYYCAKDMTTRMSQASREYD | |
| | YWGQGTLVTVSS | |
| 93 VH-4 | EVQLVESGGGLVQPGGSLRLSCAASGFTFTFRHYVMGWFRQAPGKGLEFVAAISWSG | 117 |
| | SGSYYADSVKGRFTISRDNSKNTVYLQMNSLRAEDTAVYYCAADMTTRMSQASREYD | |
| | YWGQGTLVTVSS | |
| 93 VH-5 | EVQLVESGGGLVQPGGSLRLSCAASGFTFTFRHYVMGWFRQAPGKEREFVAAISWSG | 118 |
| | SGSYYADSVKGRFTISRDNSKNTLYLQMNSLRAEDTAVYYCAKDMTTRMSQASREYD | |
| | YWGQGTLVTVSS | |
| 93 VH-6 | EVQLVESGGGLVQPGGSLRLSCAASGFTFTFRHYVMGWFRQAPGKEREFVAAISWSG | 119 |
| | SGSYYADSVKGRFTISRDNSKNTVYLQMNSLRAEDTAVYYCAADMTTRMSQASREYD | |
| | YWGQGTLVTVSS | |
| 93 VH-7 | EVQLVESGGGLVQPGGSLRLSCAASGFTFTFRHYVMGWFRQAPGKGLEFVAAISWSG | 120 |
| | SGSYYADSVKGRFTISRDNSKNTLYLQMNSLRAEDTAVYYCAADMTTRMSQASREYD | |
| | YWGQGTLVTVSS | |
| 93 VH-8 | EVQLVESGGGLVQPGGSLRLSCAASGRTFTFRHYVMGWFRQAPGKGLEFVAAISWSG | 121 |
| | SGSYYADSVKGRFTISRDNSKNTVYLQMNSLRAEDTAVYYCAADMTTRMSQASREYD | |
| | YWGQGTLVTVSS | |
| 93 VH-9 | EVQLVESGGGLVQPGGSLRLSCAASGRTFTFRHYVMGWFRQAPGKGLEFVAAISWSG | 122 |
| | SGSYYADSVKGRFTISRDNSKNTLYLQMNSLRAEDTAVYYCAADMTTRMSQASREYD | |
| | YWGQGTLVTVSS | |
| 112-VHH1- | EVQLVESGGGLVQPGGSLRLSCAASGSIFSSGTQFSDSKIDWVRQAPGKGLEWVSGI | 123 |
| PTM | FSTGSTIYEDSVKGRFTISRDNAKNSLYLQMNSLRAEDTAVYYCARIGRGILAWGQG | |
| | TLVTVSS | |
| 112-VHH2- | EVQLVESGGGLVQPGGSLRLSCAASGSIFSSGTQFSDSKIDWYRQAPGKQREWVSGI | 124 |
| PTM | FSTGSTIYEDSVKGRFTISRDNAKNSLYLQMNSLRAEDTAVYYCARIGRGILAWGQG | |
| | TLVTVSS | |
| 112-VHH3- | EVQLVESGGGLVQPGGSLRLSCAASGSIFSSGTQFSDSKIDWYRQAPGKQREWVSGI | 125 |
| PTM | FSTGSTIYEDSVKGRFTISRDNAKNSLYLQMNSLRAEDTAVYYCRVIGRGILAWGQG | |
| | TLVTVSS | |
| 112-VHH4- | EVQLVESGGGLVQPGGSLRLSCAASGSIFSSGTQFSDSKIDWYRQAPGKQREWVAGI | 126 |
| PTM | FSTGSTIYEDSVKGRFTISRDNAKNSLYLQMNSLRAEDTAVYYCRVIGRGILAWGQG | |
| | TLVTVSS | |
| 112-VHH5- | EVQLVESGGGLVQPGGSLRLSCAASGSIFSSGTQFSDSKIDWYRQAPGKQRDWVAGI | 127 |
| PTM | FSTGSTIYEDSVKGRFAISRDNAKNSLYLQMNSLRAEDTAVYYCRVIGRGILAWGQG | |
| | TLVTVSS | |
| 112-VHH6- | EVQLVESGGGLVQPGGSLRLSCAASGSIFSSGTQFSDSKIDWYRQAPGKQREWVAGI | 128 |
| PTM | FSTGSTIYEDSVKGRFTISRDNAKNMLYLQMNSLRAEDTAVYYCRVIGRGILAWGQG | |
| | TLVTVSS | |
| 112-VHH7- | EVQLVESGGGLVQPGGSLRLSCAASGSIFSSGTQFSDSKIDWYRQAPGKQREWIAGI | 129 |
| PTM | FSTGSTIYEDSVKGRFAISRDNAKNMLYLQMNSLRAEDTAVYYCRVIGRGILAWGQG | |
| | TLVTVSS | |
| 112-VH47 | EVQLVESGGGLVQPGGSLRLSCAASGFTFSSGTQFSDSKIDWYRQAPGKGLVWVAGI | 130 |
| | FSTGSTIYEDSVKGRFTISRDNAKNTGYLQMNSLRAEDTAVYYCRVIGRGILAWGQG | |
| | TLVTVSS | |

TABLE 3A
CDR sequences

| Antibody series | CDRs | SEQ ID NO: |
|---|---|---|
| 93 | RHYVMG | 55 |
| | AISWSGSGSYYADSVKG | 56 |
| | DMTTRMSQASREYDY | 57 |
| 112 | SGTQFSDSKID | 113 |
| | GIFSTGSTIYEDSVKG | 49 |
| | IGRGILA | 50 |

### Example 4. Full Kinetics of Humanized Anti-PD-L1 Monoclonal Antibodies

To explore the binding kinetics of the humanized antibody, this example further performed the full kinetic affinity testing by monitoring association and dissociation of various dose of antigen (100 nM, 50 nM, 25 nM, 12.5 nM, 6.15 nM, 3.125 nM, 1.5625 nM) against different monoclonal antibodies by Biacore. As shown Table 4, 112-VHH15-PTM affinity was comparable with ALP-Tan-3p-112 chimeric antibody. 93VH-4, 93VH-6 and 93VH-8 affinity were comparable with ALP-Tan-3p-93 chimeric antibody.

TABLE 4
Affinities

| | Kon (1/Ms) | Koff (1/s) | KD (M) |
|---|---|---|---|
| ALP-Tan-3p-112 (chimeric) | 2.14 × 10⁶ | 5.66 × 10⁻⁴ | 2.64 × 10⁻¹⁰ |
| 112-VHH3-PTM | 2.07 × 10⁶ | 4.98 × 10⁻³ | 2.40 × 10⁻⁹ |
| 112-VHH5-PTM | 1.62 × 10⁶ | 1.49 × 10⁻³ | 9.20 × 10⁻¹⁰ |
| 112-VHH7-PTM | 1.69 × 10⁶ | 3.93 × 10⁻³ | 2.32 × 10⁻⁹ |
| ALP-Tan-3p-93 (chimeric) | 1.92 × 10⁵ | 1.07 × 10⁻⁴ | 5.59 × 10⁻¹⁰ |
| 93-VH-2 | 6.15 × 10⁴ | 1.62 × 10⁻³ | 2.63 × 10⁻⁸ |
| 93-VH-3 | 4.86 × 10⁵ | 1.15 × 10⁻³ | 2.36 × 10⁻⁹ |
| 93-VH-4 | 8.05 × 10⁴ | 1.47 × 10⁻⁴ | 1.82 × 10⁻⁹ |
| 93-VH-5 | 4.52 × 10⁵ | 1.25 × 10⁻³ | 2.77 × 10⁻⁹ |
| 93-VH-6 | 9.59 × 10⁴ | 1.97 × 10⁻⁴ | 2.06 × 10⁻⁹ |
| 93-VH-7 | 8.76 × 10⁴ | 3.39 × 10⁻⁴ | 3.87 × 10⁻⁹ |
| 93-VH-8 | 1.33 × 10⁵ | 2.39 × 10⁻⁴ | 1.80 × 10⁻⁹ |
| 93-VH-9 | 1.40 × 10⁵ | 3.10 × 10⁻⁴ | 2.20 × 10⁻⁹ |

### Example 5. Binding Properties of the Humanized Anti-PD-L1 Antibody

Binding properties of the humanized anti-PD-L1 antibody of the present application were first evaluated by ELISA assay. Briefly, 100 μl anti-PD-L1 antibody 93-VH6 or 112-VH47 at different concentrations as shown in FIG. 1B was incubated in each well of 96 well plate pre-coated with human His-PD-L1, and then goat anti-human IgG Fc HRP was added and analyzed by coloring reaction of HRP with its substrate. As shown in FIG. 1B, the exemplary anti-PD-L1 antibody 93-VH6 and 112-VH47 both displayed specific binding with human PD-L1 in a dose dependent manner.

Binding capability of the anti-PD-L1 antibody of the present application was further evaluated by using Raji cells overexpressing human PD-L1. Briefly, 50 μl Raji cells overexpressing human PD-L1 were seeded into 96 well plate at a concentration of 2*10⁵ cells/well. 50 μl anti-PD-L1 antibody 93-VH6 or 112-VH47 at different concentrations as shown in FIG. 1C was added into each well and incubated with the cells on ice for 1 hour. Then the cells were washed twice by FACS buffer and supplemented with 100 μl PE-anti-hu IgG, followed by incubation on ice for 1 hour. After incubation, the cells in each well were collected and resuspended with 65 μl FACS buffer for analysis by flow cytometry. As shown in FIG. 1C, the exemplary anti-PD-L1 antibody 93-VH6 and 112-VH47 both displayed specific binding with Raji cells overexpressing human PD-L1 in a dose dependent manner.

### Example 6. T Cell Activation Bioassay (NFAT)

To test the ability of the anti-PD-L1 antibodies to stimulate T cell response, hPD-1-expressed Jurkat cells were used. Jurkat is a human T cell leukemia cell line that can activate NFAT-mediated luciferase expression upon TCR stimulation. In this assay, Jurkat cells transfected with human PD-1 gene by lentivirus were used as the responder cells. The Raji-PD-L1 cells was used as the antigen presenting cells (APC). Staphylococcal Enterotoxin E (SEE) is used to stimulate TCR signal. In this system, ectopically expressed huPD-L1 can suppress SEE stimulated NF-AT-luciferase activity in Jurkat cells, while anti-PD-L1 antibodies can reverse NFAT-luciferase activity. In short, APCs (2.5×10⁴) were co-cultured with PD-1 expressing Jurkat T cells (1×10⁵) in the presence of SEE stimulation. Anti-PD-L1 antibodies were added at the beginning of the culture. Six hours later, the resulting cells were evaluated for its luciferase activity.

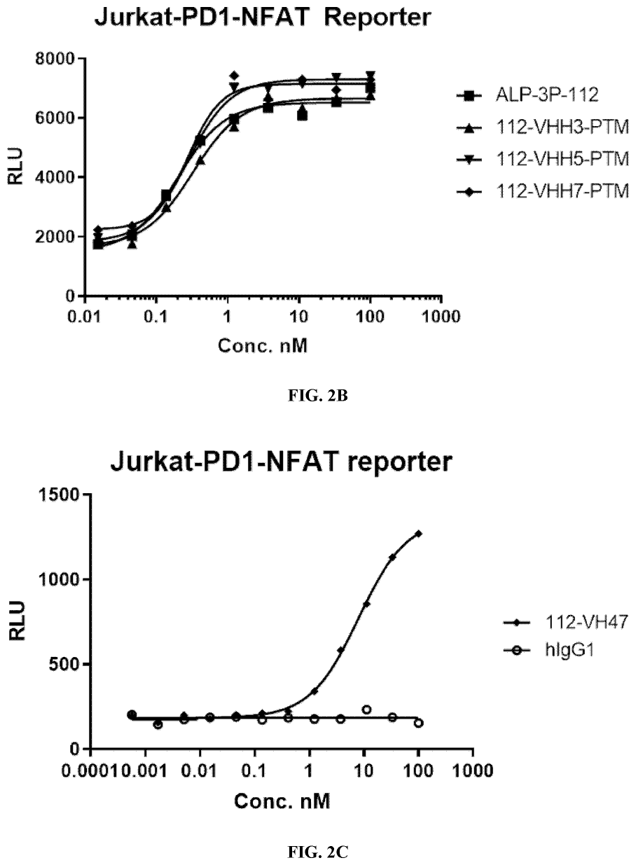

As shown in FIG. 2A-2C, all anti-PD-L1 antibodies tested blocked PD-1/PD-L1 interaction thus enhanced NFAT-mediated luciferase activity.

### Example 7. Generation of PD-L1/CD47 Bispecific Antibodies

Two previously identified anti-CD47 antibodies, 13H3 and 34C5, and anti-PD-L1 antibody, 93-VH-6 were selected to generate anti-CD47/PD-L1 bispecific antibodies in “two to two” and “one to two” formats (structures illustrated in FIG. 3).

FIG. 3A illustrates a bispecific antibody molecule of a “two to two” symmetric format. Such a bispecific antibody can include two anti-PD-L1 single domain antibodies each connected, through a GS linker, to the heavy chain of an anti-CD47 Fab which is connected to an IgG1 or IgG4 Fc.

FIG. 3B illustrates a bispecific antibody molecule of another “two to two” symmetric format. Such a bispecific antibody can include two anti-PD-L1 single domain antibodies each connected, through a GS linker, to the light chain of an anti-CD47 Fab which is connected to an IgG1 or IgG4 Fc.

FIG. 3C illustrates a bispecific antibody molecule of another “two to two” symmetric format. Such a bispecific antibody can include two anti-PD-L1 single domain antibodies each connected, through a GS linker, to CL of an IgG1 or IgG4 Fc connected to an anti-CD47 Fab.

FIG. 3D illustrates a bispecific antibody molecule of another “two to two” symmetric format. This bispecific antibody includes anti-CD47 Fab and one IgG1 Fc or one IgG4 Fc and anti-PD-L1 single domain antibody linked to CH3 via GS linker.

FIG. 3E shows a bispecific antibody molecule of a “two to one” asymmetric format. This bispecific antibody includes two tandem anti PD-L1 single domain antibodies linked by one GS linker to an IgG1 or IgG4 Fc, to which an anti-CD47 Fab is also connected. The Fc portion includes knob in hole mutations in the CH3 to reduce mispairing.

FIG. 3F shows a bispecific antibody molecule of another “two to one” asymmetric format. This bispecific antibody includes two tandem anti PD-L1 single domain antibodies linked by one GS linker to an IgG1 or IgG4 Fc, to which an anti-CD47 scFv is also connected. The Fc portion includes knob in hole mutations in the CH3 to reduce mispairing.

These bispecific antibodies were purified from 100 mL transiently transfected supernatant of the BTEK293F cells by Protein A affinity column. The purity of each of bispecific antibodies was tested with HPLC and SDS-PAGE.

TABLE 5
Parental Antibody Sequences

| Name | Sequence | SEQ ID NO: |
|---|---|---|
| Anti-CD47 | EVQLVESGGGLVKPGGSLRLSCAASGLTFERAWMNWVRQAPGKGLEWVGRI | 131 |
| 13H3-VH | KRKTDGETTDYAAPVKGRFSISRDDSKNTLYLQMNSLKTEDTAVYYCAGSN | |
| | RAFDIWGQGTMVTVSS | |
| | VH CDRs: | 135 |
| | RAWMN | |
| | RIKRKTDGETTDYAAPVKG | 136 |
| | SNRAFDI | 137 |
| Anti-CD47 | DIVMTQSPDSLAVSLGERATINCKSSQSVLYAGNNRNYLAWYQQKPGQPPK | 132 |
| 13H3-VL | LLINQASTRASGVPDRESGSGSGTEFTLIISSLQAEDVAIYYCQQYYTPPL | |
| | AFGGGTKLEIK | |
| | VL CDRs: | 138 |
| | KSSQSVLYAGNNRNYLA | 139 |
| | QASTRAS | 140 |
| | QQYYTPPLA | |
| Anti-CD47 | QVQLVQSGAEVKKPGSSVKVSCKASGYTFSSYYMHWVRQAPGQGLEWMGEI | 133 |
| 34C5-VH | NPNNARINFNEKFKTRVTLTVDKSTSTAYMELSSLRSEDTAVYYCTRGYYR | |
| | YGAWFGYWGQGTLVTVSS | |
| | VH CDRs: | 141 |
| | SYYMH | 142 |
| | EINPNNARINENEKFKT | 143 |
| | GYYRYGAWFGY | |
| Anti-CD47 | DIQMTQSPSSLSASVGDRVTITCRASQDISDYLNWYQQKPGKAPKLLIYYI | 134 |
| 34C5-VL | SRLHSGVPSRFSGSGSGTDYTLTISSLQPEDFATYYCQQGHTLPWTFGGGT | |
| | KVEIK | |
| | VL CDRs: | 144 |
| | RASQDISDYLN | 145 |
| | YISRLHS | 146 |
| | QQGHTLPWT | |
| Anti-PD- | EVQLVESGGGLVQPGGSLRLSCAASGFTFTFRHYVMGWFRQAPGKEREFVA | 119 |
| L1 93-VH- | AISWSGSGSYYADSVKGRFTISRDNSKNTVYLQMNSLRAEDTAVYYCAADM | |
| 6 | TTRMSQASREYDYWGQGTLVTVSS | |

TABLE 6
Bispecific antibodies

| | |
|---|---|
| 93VH6-13H3-H- | Heavy Chain (SEQ ID NO: 147) |
| IgG1 | EVQLVESGGGLVQPGGSLRLSCAASGFTFTFRHYVMGWFRQAPGKEREFVAAISWSGSG |
| | SYYADSVKGRFTISRDNSKNTVYLQMNSLRAEDTAVYYCAADMTTRMSQASREYDYWGQ |
| | GTLVTVSS |
| | GGGGSGGGGSGGGGSGGGGS |
| | EVQLVESGGGLVKPGGSLRLSCAASGLTFERAWMNWVRQAPGKGLEWVGRIKRKTDGET |
| | TDYAAPVKGRFSISRDDSKNTLYLQMNSLKTEDTAVYYCAGSNRAFDIWGQGTMVTVSS |
| | ASTKGPSVFPLAPSSKSTSGGTAALGCLVKDYFPEPVTVSWNSGALTSGVHTFPAVLQS |
| | SGLYSLSSVVTVPSSSLGTQTYICNVNHKPSNTKVDKKVEPKSCDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| | Light Chain (SEQ ID NO: 148) |
| | DIVMTQSPDSLAVSLGERATINCKSSQSVLYAGNNRNYLAWYQQKPGQPPKLLINQAST |
| | RASGVPDRFSGSGSGTEFTLIISSLQAEDVAIYYCQQYYTPPLAFGGGTKLEIK |
| | RTVAAPSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |
| 93VH6-34C5-H- | Heavy Chain (SEQ ID NO: 149) |
| IgG1 | EVQLVESGGGLVQPGGSLRLSCAASGFTFTFRHYVMGWFRQAPGKEREFVAAISWSGSG |
| | SYYADSVKGRFTISRDNSKNTVYLQMNSLRAEDTAVYYCAADMTTRMSQASREYDYWGQ |
| | GTLVTVSS |
| | GGGGSGGGGSGGGGSGGGGS |
| | QVQLVQSGAEVKKPGSSVKVSCKASGYTFSSYYMHWVRQAPGQGLEWMGEINPNNARIN |
| | FNEKFKTRVTLTVDKSTSTAYMELSSLRSEDTAVYYCTRGYYRYGAWFGYWGQGTLVTV |
| | SS |
| | ASTKGPSVFPLAPSSKSTSGGTAALGCLVKDYFPEPVTVSWNSGALTSGVHTFPAVLQS |
| | SGLYSLSSVVTVPSSSLGTQTYICNVNHKPSNTKVDKKVEPKSCDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| | Light Chain (SEQ ID NO: 150) |
| | DIQMTQSPSSLSASVGDRVTITCRASQDISDYLNWYQQKPGKAPKLLIYYISRLHSGVP |
| | SRFSGSGSGTDYTLTISSLQPEDFATYYCQQGHTLPWTFGGGTKVEIK |
| | RTVAAPSVFI FPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |
| 93VH6-13H3-L- | Heavy Chain (SEQ ID NO: 151) |
| IgG1 | EVQLVESGGGLVKPGGSLRLSCAASGLTFERAWMNWVRQAPGKGLEWVGRIKRKTDGET |
| | TDYAAPVKGRFSISRDDSKNTLYLQMNSLKTEDTAVYYCAGSNRAFDIWGQGTMVTVSS |
| | ASTKGPSVFPLAPSSKSTSGGTAALGCLVKDYFPEPVTVSWNSGALTSGVHTFPAVLQS |
| | SGLYSLSSVVTVPSSSLGTQTYICNVNHKPSNTKVDKKVEPKSCDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| | Light Chain (SEQ ID NO: 152) |
| | EVQLVESGGGLVQPGGSLRLSCAASGFTFTFRHYVMGWFRQAPGKEREFVAAISWSGSG |
| | SYYADSVKGRFTISRDNSKNTVYLQMNSLRAEDTAVYYCAADMTTRMSQASREYDYWGQ |
| | GTLVTVSS |
| | GGGGSGGGGSGGGGSGGGGS |
| | DIVMTQSPDSLAVSLGERATINCKSSQSVLYAGNNRNYLAWYQQKPGQPPKLLINQAST |
| | RASGVPDRFSGSGSGTEFTLIISSLQAEDVAIYYCQQYYTPPLAFGGGTKLEIK |
| | RTVAAPSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |
| 93VH6-34C5-L- | Heavy Chain (SEQ ID NO: 153) |
| IgG1 | QVQLVQSGAEVKKPGSSVKVSCKASGYTFSSYYMHWVRQAPGQGLEWMGEINPNNARIN |
| | FNEKFKTRVTLTVDKSTSTAYMELSSLRSEDTAVYYCTRGYYRYGAWFGYWGQGTLVTV |
| | SS |
| | ASTKGPSVFPLAPSSKSTSGGTAALGCLVKDYFPEPVTVSWNSGALTSGVHTFPAVLQS |
| | SGLYSLSSVVTVPSSSLGTQTYICNVNHKPSNTKVDKKVEPKSCDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| | Light Chain (SEQ ID NO: 154) |
| | EVQLVESGGGLVQPGGSLRLSCAASGFTFTFRHYVMGWFRQAPGKEREFVAAISWSGSG |
| | SYYADSVKGRFTISRDNSKNTVYLQMNSLRAEDTAVYYCAADMTTRMSQASREYDYWGQ |
| | GTLVTVSS |
| | GGGGSGGGGSGGGGSGGGGS |
| | DIQMTQSPSSLSASVGDRVTITCRASQDISDYLNWYQQKPGKAPKLLIYYISRLHSGVP |
| | SRFSGSGSGTDYTLTISSLQPEDFATYYCQQGHTLPWTFGGGTKVEIK |
| | RTVAAPSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |
| 13H3-L-93VH6- | Heavy Chain (SEQ ID NO: 155) |
| IgG1 | EVQLVESGGGLVKPGGSLRLSCAASGLTFERAWMNWVRQAPGKGLEWVGRIKRKTDGET |
| | TDYAAPVKGRFSISRDDSKNTLYLQMNSLKTEDTAVYYCAGSNRAFDIWGQGTMVTVSS |
| | ASTKGPSVFPLAPSSKSTSGGTAALGCLVKDYFPEPVTVSWNSGALTSGVHTFPAVLQS |
| | SGLYSLSSVVTVPSSSLGTQTYICNVNHKPSNTKVDKKVEPKSCDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| | Light Chain (SEQ ID NO: 156) |
| | DIVMTQSPDSLAVSLGERATINCKSSQSVLYAGNNRNYLAWYQQKPGQPPKLLINQAST |
| | RASGVPDRESGSGSGTEFTLIISSLQAEDVAIYYCQQYYTPPLAFGGGTKLEIK |
| | GGGGSGGGGSGGGGSGGGGS |
| | EVQLVESGGGLVQPGGSLRLSCAASGFTFTFRHYVMGWFRQAPGKEREFVAAISWSGSG |
| | SYYADSVKGRFTISRDNSKNTVYLQMNSLRAEDTAVYYCAADMTTRMSQASREYDYWGQ |
| | GTLVTVSS |
| | RTVAAPSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |
| 34C5-L-93VH6- | Heavy Chain (SEQ ID NO: 157) |
| IgG1 | QVQLVQSGAEVKKPGSSVKVSCKASGYTFSSYYMHWVRQAPGQGLEWMGEINPNNARIN |
| | FNEKFKTRVTLTVDKSTSTAYMELSSLRSEDTAVYYCTRGYYRYGAWFGYWGQGTLVTV |
| | SS |
| | ASTKGPSVFPLAPSSKSTSGGTAALGCLVKDYFPEPVTVSWNSGALTSGVHTFPAVLQS |
| | SGLYSLSSVVTVPSSSLGTQTYICNVNHKPSNTKVDKKVEPKSCDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| | Light Chain (SEQ ID NO: 158) |
| | DIQMTQSPSSLSASVGDRVTITCRASQDISDYLNWYQQKPGKAPKLLIYYISRLHSGVP |
| | SRFSGSGSGTDYTLTISSLQPEDFATYYCQQGHTLPWTFGGGTKVEIK |
| | GGGGSGGGGSGGGGSGGGGS |
| | EVQLVESGGGLVQPGGSLRLSCAASGFTFTFRHYVMGWFRQAPGKEREFVAAISWSGSG |
| | SYYADSVKGRFTISRDNSKNTVYLQMNSLRAEDTAVYYCAADMTTRMSQASREYDYWGQ |
| | GTLVTVSS |
| | RTVAAPSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |
| 13H3-IgG1-93VH- | Heavy Chain (SEQ ID NO: 159) |
| 6 | EVQLVESGGGLVKPGGSLRLSCAASGLTFERAWMNWVRQAPGKGLEWVGRIKRKTDGET |
| | TDYAAPVKGRFSISRDDSKNTLYLQMNSLKTEDTAVYYCAGSNRAFDIWGQGTMVTVSS |
| | ASTKGPSVFPLAPSSKSTSGGTAALGCLVKDYFPEPVTVSWNSGALTSGVHTFPAVLQS |
| | SGLYSLSSVVTVPSSSLGTQTYICNVNHKPSNTKVDKKVEPKSCDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| | GGGGSGGGGSGGGGSGGGGS |
| | EVQLVESGGGLVQPGGSLRLSCAASGFTFTFRHYVMGWFRQAPGKEREFVAAISWSGSG |
| | SYYADSVKGRFTISRDNSKNTVYLQMNSLRAEDTAVYYCAADMTTRMSQASREYDYWGQ |
| | GTLVTVSS |
| | Light Chain (SEQ ID NO: 160) |
| | DIVMTQSPDSLAVSLGERATINCKSSQSVLYAGNNRNYLAWYQQKPGQPPKLLINQAST |
| | RASGVPDRFSGSGSGTEFTLIISSLQAEDVAIYYCQQYYTPPLAFGGGTKLEIK |
| | RTVAAPSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |
| 34C5-IgG1-93VH- | Heavy Chain (SEQ ID NO: 161) |
| 6 | QVQLVQSGAEVKKPGSSVKVSCKASGYTESSYYMHWVRQAPGQGLEWMGEINPNNARIN |
| | FNEKFKTRVTLTVDKSTSTAYMELSSLRSEDTAVYYCTRGYYRYGAWFGYWGQGTLVTV |
| | SS |
| | ASTKGPSVFPLAPSSKSTSGGTAALGCLVKDYFPEPVTVSWNSGALTSGVHTFPAVLQS |
| | SGLYSLSSVVTVPSSSLGTQTYICNVNHKPSNTKVDKKVEPKSCDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| | GGGGSGGGGSGGGGSGGGGS |
| | EVQLVESGGGLVQPGGSLRLSCAASGFTFTFRHYVMGWFRQAPGKEREFVAAISWSGSG |
| | SYYADSVKGRFTISRDNSKNTVYLQMNSLRAEDTAVYYCAADMTTRMSQASREYDYWGQ |
| | GTLVTVSS |
| | Light Chain (SEQ ID NO: 162) |
| | DIQMTQSPSSLSASVGDRVTITCRASQDISDYLNWYQQKPGKAPKLLIYYISRLHSGVP |
| | SRESGSGSGTDYTLTISSLQPEDFATYYCQQGHTLPWTFGGGTKVEIK |
| | RTVAAPSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |
| 93VH6-34C5 IgG1 | Heavy Chain-1 (SEQ ID NO: 163) |
| KIH | EVQLVESGGGLVQPGGSLRLSCAASGFTFTFRHYVMGWFRQAPGKEREFVAAISWSGSG |
| | SYYADSVKGRFTISRDNSKNTVYLQMNSLRAEDTAVYYCAADMTTRMSQASREYDYWGQ |
| | GTLVTVSS |
| | GGGGSGGGGSGGGGSGGGGS |
| | EVQLVESGGGLVQPGGSLRLSCAASGFTFTFRHYVMGWFRQAPGKEREFVAAISWSGSG |
| | SYYADSVKGRFTISRDNSKNTVYLQMNSLRAEDTAVYYCAADMTTRMSQASREYDYWGQ |
| | GTLVTVSS |
| | DKTHTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKTISK |
| | AKGQPREPQVYTLPPSREEMTKNQVSLWCLVKGFYPSDIAVEWESNGQPENNYKTTPPV |
| | LDSDGSFFLYSKLTVDKSRWQQGNVFSCSVMHEALHNHYTQKSLSLSPGK |
| | Heavy Chain-2 (SEQ ID NO: 164) |
| | QVQLVQSGAEVKKPGSSVKVSCKASGYTFSSYYMHWVRQAPGQGLEWMGEINPNNARIN |
| | FNEKFKTRVTLTVDKSTSTAYMELSSLRSEDTAVYYCTRGYYRYGAWFGYWGQGTLVTV |
| | SS |
| | ASTKGPSVFPLAPSSKSTSGGTAALGCLVKDYFPEPVTVSWNSGALTSGVHTFPAVLQS |
| | SGLYSLSSVVTVPSSSLGTQTYICNVNHKPSNTKVDKKVEPKSCDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLSCAVKGFYPSDIAVEWESNGQPENNYKTTPPVLDSDGSFFLVSKLTV |
| | DKSRWQQGNVFSCSVMHEALHNHYTQKSLSLSPGK |
| | Light Chain (SEQ ID NO: 165) |
| | DIQMTQSPSSLSASVGDRVTITCRASQDISDYLNWYQQKPGKAPKLLIYYISRLHSGVP |
| | SRFSGSGSGTDYTLTISSLQPEDFATYYCQQGHTLPWTFGGGTKVEIK |
| | RTVAAPSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |
| 93VH6-34C5 IgG1 | Heavy Chain-1 (SEQ ID NO: 166) |
| KIH-2 | EVQLVESGGGLVQPGGSLRLSCAASGFTFTFRHYVMGWFRQAPGKEREFVAAISWSGSG |
| | SYYADSVKGRFTISRDNSKNTVYLQMNSLRAEDTAVYYCAADMTTRMSQASREYDYWGQ |
| | GTLVTVSS |
| | GGGGSGGGGGGGGSGGGGS |
| | EVQLVESGGGLVQPGGSLRLSCAASGFTFTFRHYVMGWFRQAPGKEREFVAAISWSGSG |
| | SYYADSVKGRFTISRDNSKNTVYLQMNSLRAEDTAVYYCAADMTTRMSQASREYDYWGQ |
| | GTLVTVSS |
| | DKTHTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKTISK |
| | AKGQPREPQVYTLPPSREEMTKNQVSLSCAVKGFYPSDIAVEWESNGQPENNYKTTPPV |
| | LDSDGSFFLVSKLTVDKSRWQQGNVFSCSVMHEALHNHYTQKSLSLSPGK |
| | Heavy Chain-2 (SEQ ID NO: 167) |
| | QVQLVQSGAEVKKPGSSVKVSCKASGYTFSSYYMHWVRQAPGQGLEWMGEINPNNARIN |
| | FNEKFKTRVTLTVDKSTSTAYMELSSLRSEDTAVYYCTRGYYRYGAWFGYWGQGTLVTV |
| | SS |
| | ASTKGPSVFPLAPSSKSTSGGTAALGCLVKDYFPEPVTVSWNSGALTSGVHTFPAVLQS |
| | SGLYSLSSVVTVPSSSLGTQTYICNVNHKPSNTKVDKKVEPKSC |
| | DKTHTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKTISK |
| | AKGQPREPQVYTLPPSREEMTKNQVSLWCLVKGFYPSDIAVEWESNGQPENNYKTTPPV |
| | LDSDGSFFLYSKLTVDKSRWQQGNVFSCSVMHEALHNHYTQKSLSLSPGK |
| | Light Chain (SEQ ID NO: 168) |
| | DIQMTQSPSSLSASVGDRVTITCRASQDISDYLNWYQQKPGKAPKLLIYYISRLHSGVP |
| | SRFSGSGSGTDYTLTISSLQPEDFATYYCQQGHTLPWTFGGGTKVEIK |
| | RTVAAPSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |
| 93VH6-34C5 scFv | Heavy Chain-1 (SEQ ID NO: 169) |
| IgG1 KIH | EVQLVESGGGLVQPGGSLRLSCAASGFTFTFRHYVMGWFRQAPGKEREFVAAISWSGSG |
| | SYYADSVKGRFTISRDNSKNTVYLQMNSLRAEDTAVYYCAADMTTRMSQASREYDYWGQ |
| | GTLVTVSS |
| | GGGGSGGGGSGGGGSGGGGS |
| | EVQLVESGGGLVQPGGSLRLSCAASGFTFTFRHYVMGWFRQAPGKEREFVAAISWSGSG |
| | SYYADSVKGRFTISRDNSKNTVYLQMNSLRAEDTAVYYCAADMTTRMSQASREYDYWGQ |
| | GTLVTVSS |
| | DKTHTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKTISK |
| | AKGQPREPQVYTLPPSREEMTKNQVSLWCLVKGFYPSDIAVEWESNGQPENNYKTTPPV |
| | LDSDGSFFLYSKLTVDKSRWQQGNVFSCSVMHEALHNHYTQKSLSLSPGK |
| | Heavy Chain-2 (SEQ ID NO: 170) |
| | QVQLVQSGAEVKKPGSSVKVSCKASGYTFSSYYMHWVRQAPGQGLEWMGEINPNNARIN |
| | FNEKFKTRVTLTVDKSTSTAYMELSSLRSEDTAVYYCTRGYYRYGAWFGYWGQGTLVTV |
| | SS |
| | GGGGSGGGGSGGGGS |
| | DIQMTQSPSSLSASVGDRVTITCRASQDISDYLNWYQQKPGKAPKLLIYYISRLHSGVP |
| | SRFSGSGSGTDYTLTISSLQPEDFATYYCQQGHTLPWTFGGGTKVEIK |
| | DKTHTCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKTISK |
| | AKGQPREPQVYTLPPSREEMTKNQVSLSCAVKGFYPSDIAVEWESNGQPENNYKTTPPV |
| | LDSDGSFFLVSKLTVDKSRWQQGNVFSCSVMHEALHNHYTQKSLSLSPGK |

### Example 8. Anti-CD47/PD-L1 Bispecific Antibodies Blocking the Binding of CD47 to SIRPα

The assay was conducted according to the description of CD47/SIRPα Binding Assay Kit (Cisbio). In brief, serially diluted antibodies, Tag1-CD47 and Tag2-SIRPa were premixed and incubated for 15 min at room temp, then the premixed anti-Tag1-Tb3 and anti-Tag2-XL665 was added and incubated for 1 hour at RT. Fluorescence data were read on a PerkinElmer Envision plate reader using laser as light source. The anti-CD47 antibody (13H3 or 34C5) was used as positive control in this study.

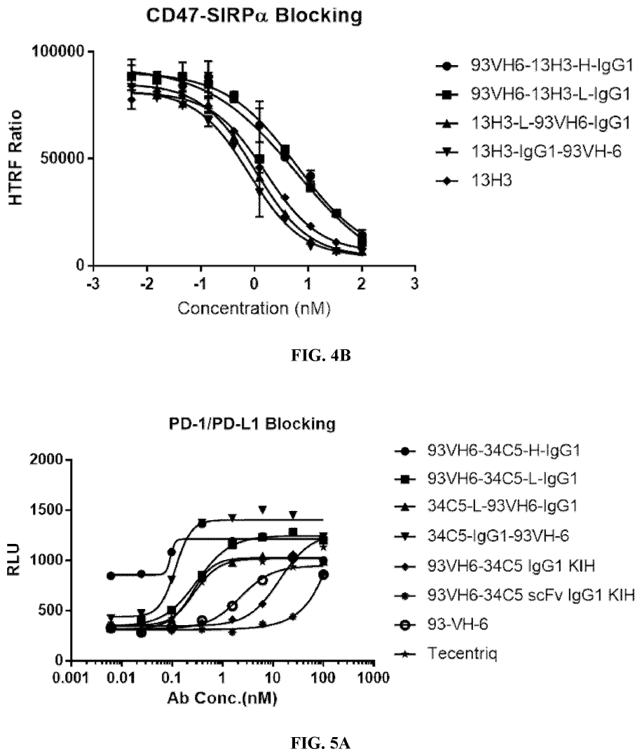

Results are presented in FIGS. 4A and 4B, which show that 34C5 had stronger blocking activity than 13H3. Moreover, 34C5-IgG1-93VH-6, 93VH6-13H3-H-IgG1 and 93VH6-13H3-L-IgG1 had some activity losses, compared to the parental anti-CD47 monoclonal antibodies. The remaining PD-L1/CD47 bispecific antibodies, however, had comparable or even stronger SIRPα blocking activity to their parental anti-CD47 antibodies.

### Example 9. T Cell Activation Bioassay (NFAT)

To test the ability of the anti-CD47/PD-L1 bispecific antibodies to stimulate T cell response, hPD-1-expressed Jurkat cells were used. Jurkat is a human T cell leukemia cell line that can activate NF-AT activated luciferase expression upon TCR stimulation. In this assay, Jurkat cells transfected with human PD-1 gene by lentivirus were used as the responder cells. The Raji-PD-L1 cells was used as the antigen presenting cells (APC). Staphylococcal Enterotoxin E (SEE) is used to stimulate TCR signal. In this system, ectopically expressed huPD-L1 can suppress SEE stimulated NF-AT-luciferase activity in Jurkat cells, while anti-PD-L1 antibodies can reverse NF-AT-luciferase activity. In short, APCs (2.5×10⁴) were co-cultured with PD-1 expressing Jurkat T cells (1×10⁵) in the presence of SEE stimulation. Anti-PD-L1 antibodies were added at the beginning of the culture. Six hours later, the resulting cells were evaluated for its luciferase activity.

As shown in FIG. 5, all bispecific antibodies which are in 2 to 2 format had comparable or stronger efficacy in blocking PD-1/PD-L1 mediated NF-AT-luciferase activity, compared with parental PD-L1 monoclonal antibody 93-VH-6.

### Example 10. Anti-CD47/PD-L1 Bispecific Antibodies Showed Increased Phagocytosis of Tumor Cells by Human Macrophage (MΦ)

Monocytes were isolated from human blood, and the monocytes were differentiated into macrophages in the presence of hGCSF for 6 days. The monocyte derived macrophages (MDMs) were scraped and re-plated in 24-well dishes and allowed to adhere for 24 hours. The human tumor cell line RKO was chosen as target cells and labeled with 1 mM CellTrace-Far red for 20 minutes, and MDMs were labeled with 1 mM Cell Trace-Violet for 20 minutes, then mixed at a ratio of 3:1 tumor cells per phagocyte and anti-CD47/PD-L1 bispecific antibodies and corresponding control mAb and combination were added at various doses. After incubation for 3 hours, phagocytosis of the target cell was analyzed by flow cytometry. Phagocytosis was measured by gating on macrophage and then assessing the percent of double positive cells.

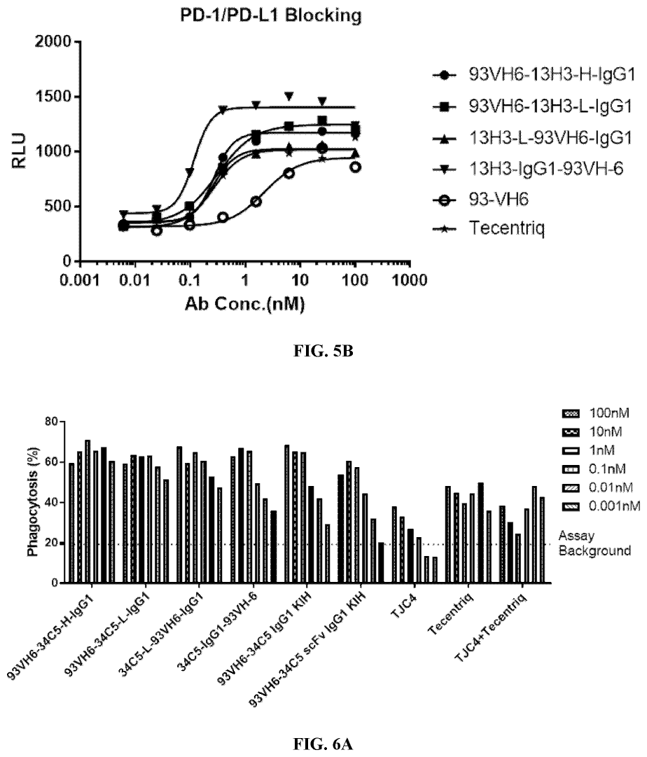

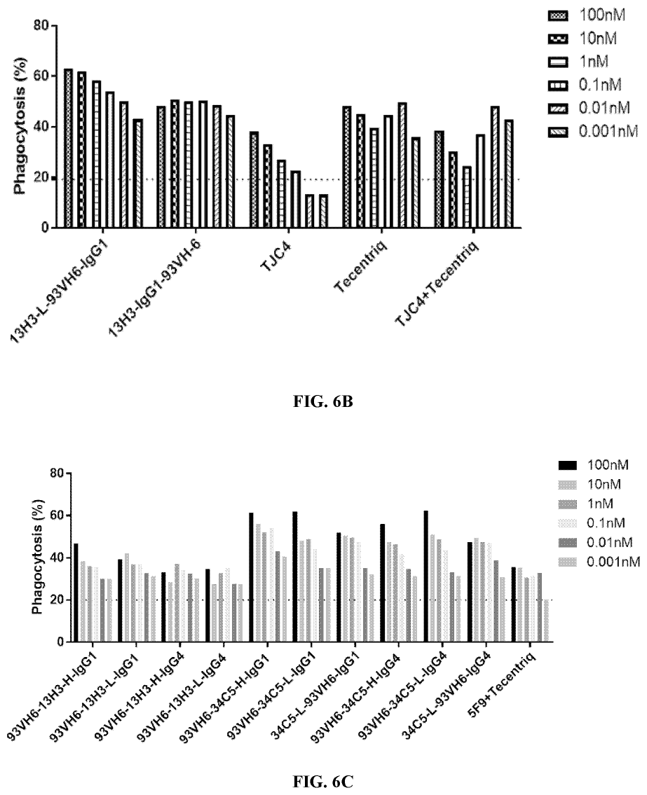

As shown in FIGS. 6A and 6B, anti-CD47/PD-L1 bispecific antibodies exhibited higher ADCP efficacy than the combination treatment of parental monoclonal antibodies and clinical benchmark antibodies.

Another study was conducted to further test whether the isotype effects the ADCP efficacy. This study compared the differences between bispecific antibodies with hIgG1 Fc and hIgG4 Fc respectively in ADCP assay. Results are shown in FIG. 6C. ADCP efficacy of anti-CD47/PD-L1 bispecific antibody were overall comparable when the isotype changed from hIgG1 to hIgG4.

### Example 11. RKO Cell-Based Binding of Anti-CD47/PD-L1 Bispecific Antibodies

RKO cells are human colon carcinoma cell lines that express endogenous level of human CD47 and human PD-L1 on the surface. RKO cells were incubated with serial diluted anti-CD47/PD-L1 bispecific antibodies, parental CD47 or PD-L1 monospecific antibodies at 4° C. for 30 minutes. Then cells were washed with FACS buffer three times, followed by incubation with APC-labeled secondary antibody at 4° C. for 30 minutes. Then cells were washed with FACS buffer for three times. Binding was measured by flow cytometry.

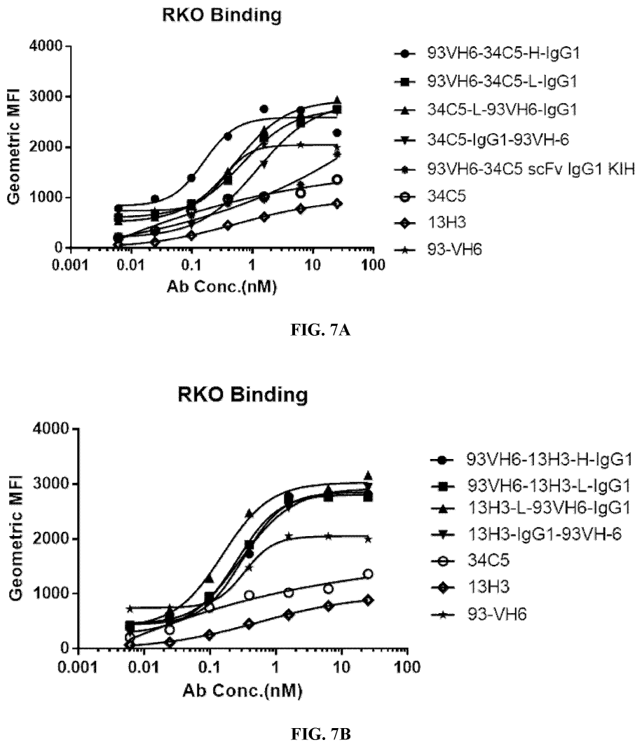

As shown in FIGS. 7A and 7B, the anti-CD47/PD-L1 bispecific antibodies in a symmetric format showed either stronger or comparable binding capability than parental PD-L1 monospecific antibodies.

### Example 12. RBC Binding and RBC Agglutination Test of Anti-CD47/PD-L1 Bispecific Antibodies

### 10.1 RBC Binding Assay

Human RBCs were diluted to 1% in PBS and incubated with anti-CD47/PD-L1 bispecific antibodies (antibody titration started from 200 nM and 3-fold titrated down) at 4° C. for 1 hour, followed by the addition of PE-conjugated secondary antibody at 4° C. for 30 minutes. Binding of anti-CD47/PD-L1 antibodies against human RBCs was examined by flow cytometry.

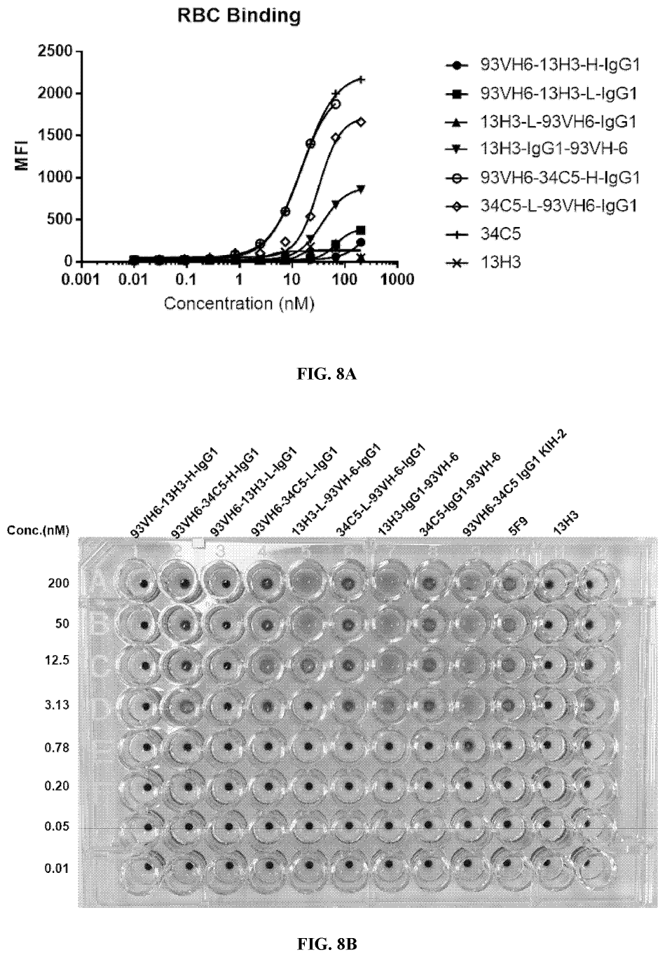

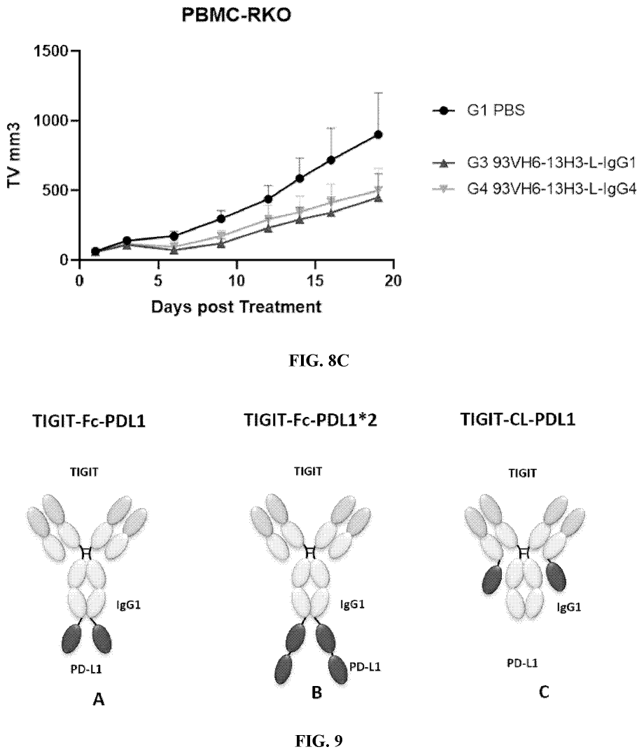

As shown in FIG. 8A, 93VH6-13H3-H-IgG1, 93VH6-13H3-L-IgG1 and 13H3-L-93VH6-IgG1 showed minimal or no RBC binding, comparable to 13H3 antibody. Among all antibodies tested, parental CD47 antibody 34C5 showed strongest RBC binding.

### 10.2 RBC Agglutination Assay

Human RBCs were diluted to 1% in PBS and incubated at room temperature for 2 hours with a titration of anti-CD47/PD-L1 antibody (antibody titration start form 200 nM and 3-4-fold titrated down) in a round bottom 96-well plate. Evidence of hemagglutination is demonstrated by the presence of non-settled RBCs, appearing as a haze compared to a punctuate red dot of non-hemagglutinated RBCs.

As shown in FIG. 8B, 93VH6-13H3-H-IgG1 and 93VH6-13H3-L-IgG1 showed no appreciably RBC agglutination, which is similar to parental CD47 antibody 13H3. Reference antibody 5F9 showed RBC agglutination at 4 tested concentrations.

### Example 13. In Vivo Anti-Tumor Efficacy of Anti-CD47/PD-L1 Bispecific Antibodies

12 NOG mice were individually injected with human PBMC in 0.2 mL DPBS (i.v., 5×10⁶/mouse). After 8 days, 1×10⁶ RKO cells were inoculated subcutaneously at the right flank of the mice. When the mean tumor size reached 57 mm³, tumor-bearing mice were randomly divided into three groups, 4 mice per group, and intraperitoneally administrated with PBS, 93VH6-13H3-L-IgG1 (12 mg/kg), 93VH6-13H3-L-IgG4 (12 mg/kg), respectively. The tumor volume and body weight were measured and recorded twice per week. On day 19, animals were euthanized. As shown in FIG. 8C, 93VH6-13H3-L-IgG1 and 93VH6-13H3-L-IgG4 treatment showed significantly inhibited tumor growth as compared with PBS, suggesting potent anti-tumor efficacy of the anti-CD47/PD-L1 bsAbs of the present application.

### Example 14. Generation of PD-L1/TIGIT Bispecific Antibodies

The exemplary anti-PD-L1 single domain antibodies (sdAb) 93-VH6 and 112-VH47 were selected to generate anti-PD-L1/TIGIT bispecific antibody in different formats (structure illustrated in FIG. 9).

In one format, two PD-L1 sdAb were fused to the C-terminus of the heavy chains of the anti-TIGIT portion (referred as TIGIT-Fc-PD-L1) or the C-terminus of the light chains of the anti-TIGIT portion (referred as TIGIT-CL-PD-L1) through a G4S linker. Alternatively, four PD-L1 sdAb, two in each tandem group, were linked through a G4S linker to the C-terminus of the heavy chains of the TIGIT portion (referred as TIGIT-Fc-PD-L1*2).

These bispecific antibodies were purified from 100 mL supernatant of transiently transfected HEK293F cells culture by Protein A affinity column. The purity of each of bispecific antibodies was confirmed with HPLC and SDS-PAGE.

TABLE 7
Parental Antibody Sequences

| Name | Sequences | SEQ ID NO: |
|---|---|---|
| TIGIT Ab | EVKLVESGGGLVQPGGSLRLSCAASGFTFSDYYMYWVRQAPGKRLEWVASIT | 171 |
| VH | KGGGSTYYPDTLKGRFTISRDNAKNSLYLQMNRLRAEDTAVYYCARQSSYDF | |
| | VMDYWGQGTTVTVSS | |
| | VH CDRs: | 173 |
| | DYYMY | 174 |
| | SITKGGGSTYYPDTLKG | 175 |
| | QSSYDFVMDY | |
| TIGIT Ab | DIVMTQSPSSLSASVGDRVTITCKASQDVDTAVAWYQQKPGKAPKLLIYWAS | 172 |
| VL | ARHTGVPSRFSGSGSGTDFTFTISSLQPEDIATYYCQQYSNYPLTFGQGTKL | |
| | EIK | |
| | VL CDRs: | 176 |
| | KASQDVDTAVA | 177 |
| | WASARHT | 178 |
| | QQYSNYPLT | |
| PD-L1 Ab | EVQLVESGGGLVQPGGSLRLSCAASGFTFTFRHYVMGWFRQAPGKEREFVAA | 119 |
| (93-VH6) | ISWSGSGSYYADSVKGRFTISRDNSKNTVYLQMNSLRAEDTAVYYCAADMTT | |
| | RMSQASREYDYWGQGTLVTVSS | |
| PD-L1 Ab | EVQLVESGGGLVQPGGSLRLSCAASGFTFSSGTQFSDSKIDWYRQAPGKGLV | 130 |
| (112-VH47) | WVAGIFSTGSTIYEDSVKGRFTISRDNAKNTGYLQMNSLRAEDTAVYYCRVI | |
| | GRGILAWGQGTLVTVSS | |

TABLE 8
PD-L1/TIGIT bsAb

| | |
|---|---|
| TIGIT-Fc-93-VH6 | Heavy Chain (SEQ ID NO: 179) |
| | EVKLVESGGGLVQPGGSLRLSCAASGFTFSDYYMYWVRQAPGKRLEWVASITKGGGSTY |
| | YPDTLKGRFTISRDNAKNSLYLQMNRLRAEDTAVYYCARQSSYDFVMDYWGQGTTVTVS |
| | S |
| | ASTKGPSVFPLAPSSKSTSGGTAALGCLVKDYFPEPVTVSWNSGALTSGVHTFPAVLQS |
| | SGLYSLSSVVTVPSSSLGTQTYICNVNHKPSNTKVDKKVEPKSCDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| | GGGGSGGGGSGGGGSGGGGS |
| | EVQLVESGGGLVQPGGSLRLSCAASGFTFTFRHYVMGWFRQAPGKEREFVAAISWSGSG |
| | SYYADSVKGRFTISRDNSKNTVYLQMNSLRAEDTAVYYCAADMTTRMSQASREYDYWGQ |
| | GTLVTVSS |
| | Light Chain (SEQ ID NO: 180) |
| | DIVMTQSPSSLSASVGDRVTITCKASQDVDTAVAWYQQKPGKAPKLLIYWASARHTGVP |
| | SRFSGSGSGTDFTFTISSLQPEDIATYYCQQYSNYPLTFGQGTKLEIK |
| | RTVAAPSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |
| TIGIT-Fc-93- | Heavy Chain (SEQ ID NO: 181) |
| VH6*2 | EVKLVESGGGLVQPGGSLRLSCAASGFTFSDYYMYWVRQAPGKRLEWVASITKGGGSTY |
| | YPDTLKGRFTISRDNAKNSLYLQMNRLRAEDTAVYYCARQSSYDFVMDYWGQGTTVTVS |
| | S |
| | ASTKGPSVFPLAPSSKSTSGGTAALGCLVKDYFPEPVTVSWNSGALTSGVHTFPAVLQS |
| | SGLYSLSSVVTVPSSSLGTQTYICNVNHKPSNTKVDKKVEPKSCDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| | GGGGSGGGGSGGGGSGGGGS |
| | EVQLVESGGGLVQPGGSLRLSCAASGFTFTFRHYVMGWFRQAPGKEREFVAAISWSGSG |
| | SYYADSVKGRFTISRDNSKNTVYLQMNSLRAEDTAVYYCAADMTTRMSQASREYDYWGQ |
| | GTLVTVSS |
| | GGGGSGGGGSGGGGSGGGGS |
| | EVQLVESGGGLVQPGGSLRLSCAASGFTFTFRHYVMGWFRQAPGKEREFVAAISWSGSG |
| | SYYADSVKGRFTISRDNSKNTVYLQMNSLRAEDTAVYYCAADMTTRMSQASREYDYWGQ |
| | GTLVTVSS |
| | Light Chain (SEQ ID NO: 180) |
| | DIVMTQSPSSLSASVGDRVTITCKASQDVDTAVAWYQQKPGKAPKLLIYWASARHTGVP |
| | SRFSGSGSGTDFTFTISSLQPEDIATYYCQQYSNYPLTFGQGTKLEIK |
| | RTVAAPSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |
| TIGIT-CL-93-VH6 | Heavy Chain (SEQ ID NO: 182) |
| | EVKLVESGGGLVQPGGSLRLSCAASGFTFSDYYMYWVRQAPGKRLEWVASITKGGGSTY |
| | YPDTLKGRFTISRDNAKNSLYLQMNRLRAEDTAVYYCARQSSYDFVMDYWGQGTTVTVS |
| | S |
| | ASTKGPSVFPLAPSSKSTSGGTAALGCLVKDYFPEPVTVSWNSGALTSGVHTFPAVLQS |
| | SGLYSLSSVVTVPSSSLGTQTYICNVNHKPSNTKVDKKVEPKSCDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| | Light Chain (SEQ ID NO: 183) |
| | DIVMTQSPSSLSASVGDRVTITCKASQDVDTAVAWYQQKPGKAPKLLIYWASARHTGVP |
| | SRFSGSGSGTDFTFTISSLQPEDIATYYCQQYSNYPLTFGQGTKLEIK |
| | RTVAAPSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |
| | GGGGSGGGGSGGGGSGGGGS |
| | EVQLVESGGGLVQPGGSLRLSCAASGFTFTFRHYVMGWFRQAPGKEREFVAAISWSGSG |
| | SYYADSVKGRFTISRDNSKNTVYLQMNSLRAEDTAVYYCAADMTTRMSQASREYDYWGQ |
| | GTLVTVSS |
| TIGIT-Fc-112- | Heavy Chain (SEQ ID NO: 184) |
| VH47 | EVKLVESGGGLVQPGGSLRLSCAASGFTFSDYYMYWVRQAPGKRLEWVASITKGGGSTY |
| | YPDTLKGRFTISRDNAKNSLYLQMNRLRAEDTAVYYCARQSSYDFVMDYWGQGTTVTVS |
| | S |
| | ASTKGPSVFPLAPSSKSTSGGTAALGCLVKDYFPEPVTVSWNSGALTSGVHTFPAVLQS |
| | SGLYSLSSVVTVPSSSLGTQTYICNVNHKPSNTKVDKKVEPKSCDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| | GGGGSGGGGSGGGGSGGGGS |
| | EVQLVESGGGLVQPGGSLRLSCAASGFTFSSGTQFSDSKIDWYRQAPGKGLVWVAGIFS |
| | TGSTIYEDSVKGRFTISRDNAKNTGYLQMNSLRAEDTAVYYCRVIGRGILAWGQGTLVT |
| | VSS |
| | Light Chain (SEQ ID NO: 180) |
| | DIVMTQSPSSLSASVGDRVTITCKASQDVDTAVAWYQQKPGKAPKLLIYWASARHTGVP |
| | SRFSGSGSGTDFTFTISSLQPEDIATYYCQQYSNYPLTFGQGTKLEIK |
| | RTVAAPSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |

### Example 15. Binding Properties of PD-L1/TIGIT bsAb to PD-L1

The binding affinity of the PD-L1/TIGIT bsAb to recombinant human his-tagged PD-L1 protein was tested by BIACORE®. The PD-L1/TIGIT bsAb molecules or the parental anti-PD-L1 sdAb were captured by protein A chip. A series of dilutions of human PD-L1 protein (6.25 nM-100 nM) were injected over captured antibody at a flow rate of 10 μL/min. The antigen was allowed to associate for 180 s and dissociate for 1200 s. All the experiments were carried out on a Biacore T200. Data analysis was carried out using Biacore T200 evaluation software.

The data show that the PD-L1 binding affinity was not compromised in the PD-L1/TIGIT bsAb molecules when compared with its parental anti-PD-L1 antibody (Table 9).

TABLE 9
Affinity of PD-L1/TIGIT bsAb molecules to human PD-L1

| | Analyte | | |
|---|---|---|---|
| | hPD-L1-his | | |
| Ligand | ka (1/Ms) | kd (1/s) | KD (M) |
| PD-L1 sdAb | 3.526E+4 | 1.283E−4 | 3.638E−9 |
| TIGIT-Fc-93-VH6 | 3.708E+4 | 4.247E−5 | 1.145E−9 |
| TIGIT-Fc-93-VH6*2 | 2.189E+4 | 1.183E−4 | 5.403E−9 |
| TIGIT-CL-93-VH6 | 3.592E+4 | 1.426E−4 | 3.970E−9 |

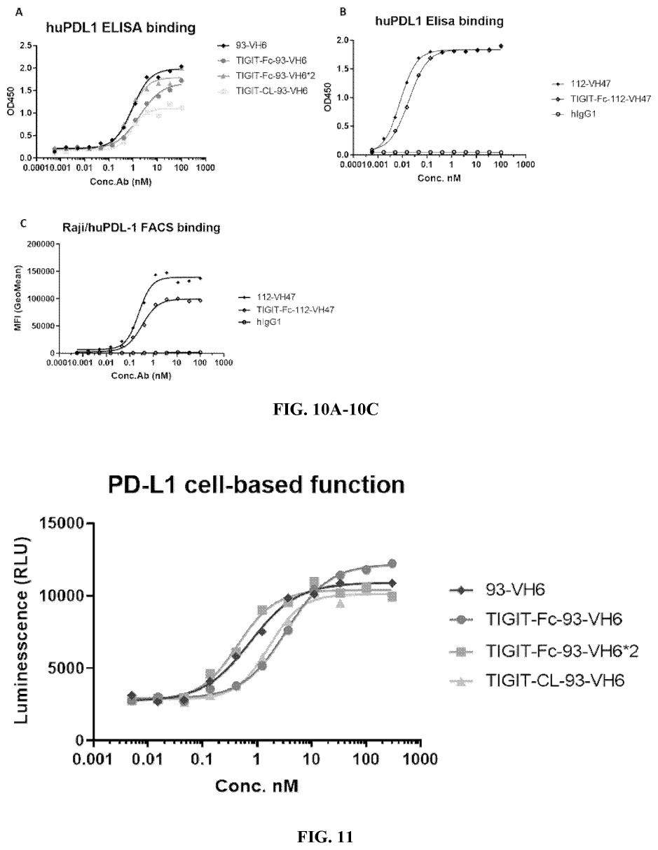

Binding of the anti-PD-L1/TIGIT bsAb molecules with human PD-L1 was further analyzed by ELISA. Briefly, 100 μl anti-PD-L1/TIGIT bsAb TIGIT-Fc-93-VH6, TIGIT-Fc-93-VH6*2, TIGIT-CL-93-VH6 and TIGIT-Fc-112-VH47 at different concentrations as shown in FIG. 10A and FIG. 10B were incubated in each well of 96 well plate pre-coated with human His-PD-L1, and then the binding between the anti-PD-L1/TIGIT bsAbs and the human His-PD-L1 was analyzed via goat anti-human IgG Fc HRP. As shown in FIG. 10A and FIG. 10B, the tested PD-L1/TIGIT bsAbs including TIGIT-Fc-93-VH6, TIGIT-Fc-93-VH6*2, TIGIT-CL-93-VH6 and TIGIT-Fc-112-VH47 all displayed specific binding with human PD-L1 in a dose dependent manner.

Furthermore, binding capability of the anti-PD-L1/TIGIT bsAbs of the present application with cells expressing PD-L1 was analyzed by using Raji-PD-L1 cells. Briefly, 50 μl Raji cells overexpressing human PD-L1 were seeded into 96 well plate with 2*10⁵ cells/well. 50 μl anti-PD-L1 antibody 112-VH47 or TIGIT-Fc-112-VH47 at different concentrations as shown in FIG. 10C was added into each well and incubated with the cells on ice for 1 hour. Then the cells were washed twice by FACS buffer and supplemented with 100 μl PE-anti-hu IgG, followed by incubation on ice for 1 hour After incubation, the cells in each well were collected and resuspended in 65 μl FACS buffer for analysis by flow cytometry As shown in FIG. 10C, TIGIT-Fc-112-VH47 displayed specific binding with Raji cells expressing human PD-L1 in a dose dependent manner.

### Example 16. PD-L1 Antagonist Activity of PD-L1/TIGIT bsAb Molecules

To evaluate the PD-L1 antagonistic activity of the PD-L1/TIGIT bsAb molecules, PD-L1 cell-based functional assay was performed as described in Example 6.

As shown in FIG. 11, TIGIT-Fc-93-VH6*2 bsAb molecule showed comparable antagonistic activity with the parental 93-VH6 sdAb. TIGIT-Fc-93-VH6 showed enhanced maximum effect but reduced EC50 in PD-L1 antagonist activity when compared with anti-93-VH6 sdAb. TIGIT-CL-93-VH6 bsAb showed comparable maximum effect but reduced EC50 in PD-L1 antagonist activity when compared with 93-VH6 sdAb.

### Example 17. Binding Properties of PD-L1/TIGIT bsAb to TIGIT

The binding of PD-L1/TIGIT bsAbs and the parental TIGIT antibody to recombinant His-tagged human TIGIT-ECD protein was examined by Biacore T200. The antibodies were captured by Protein A chip. Serial concentrations of His-tagged human TIGIT-ECD protein (0.78 nM-12.5 nM) were injected over capture antibodies at the flow rate of 10 μl/min. The association phase was 180 s and the dissociation phase was 1200 s.

The results are shown in Table 10 below. The Biacore results for the PD-L1/TIGIT antibodies have shown that these bispecific antibodies are high-affinity binders to human TIGIT. As shown in the table, the PD-L1/TIGIT antibodies had comparable affinity to their parental TIGIT antibodies.

TABLE 10
Affinity of anti-PD-L1/TIGIT bsAb molecules to human TIGIT

Analyte
hTIGIT-his

| Ligand | ka (1/Ms) | kd (1/s) | KD (M) |
|---|---|---|---|
| TIGIT antibody | 1.190E+6 | 1.716E−4 | 1.442E−10 |
| TIGIT-Fc-93-VH6 | 1.243E+6 | 1.392E−4 | 1.119E−10 |
| TIGIT-Fc-93-VH6*2 | 1.433E+6 | 1.307E−4 | 9.117E−11 |
| TIGIT-CL-93-VH6 | 1.715E+6 | 1.759E−4 | 1.026E−10 |

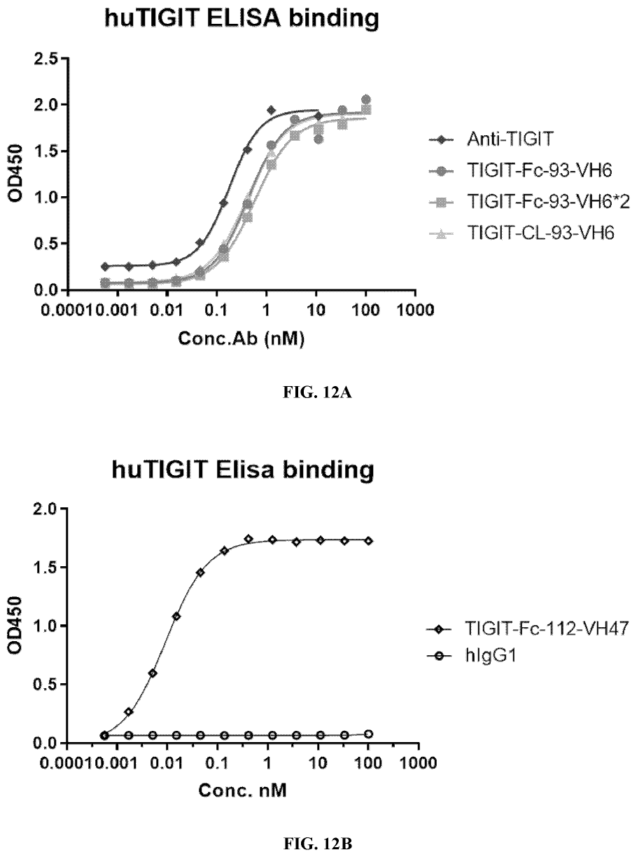

To evaluate the binding capability to TIGIT protein, the PD-L1/TIGIT bsAbs were subjected to ELISA binding test for His-tagged human TIGIT. As shown in FIG. 12, all the tested PD-L1/TIGIT bsAbs displayed specific binding with TIGIT in a dose dependent manner.

### Example 18. TIGIT Antagonist Activity of Properties of PD-L1/TIGIT bsAbs

To evaluate the TIGIT-blocking function of PD-L1/TIGIT bsAb antibodies, in vitro Jurkat cell-based functional assay was used. In brief, human TIGIT and it counter-receptor CD226 were simultaneously overexpressed on Jurkat T cells, while their co-ligand human CD155 was overexpressed on Raji cells. When these two cell types were cocultured in the presence of super antigen, the negative signaling delivered on Jurkat cells by TIGIT-CD155 ligation inhibit Jurkat cell activation. A similar luciferase report system as PD-L1 blocking assay was used to assess the activation status of Jurkat cells. When serial diluted PD-L1/TIGIT bsAbs or anti-TIGIT antibodies were added to the culture systems, antibodies can dose-dependently enhance luciferase expression of Jurkat-TIGIT-CD226 cells.

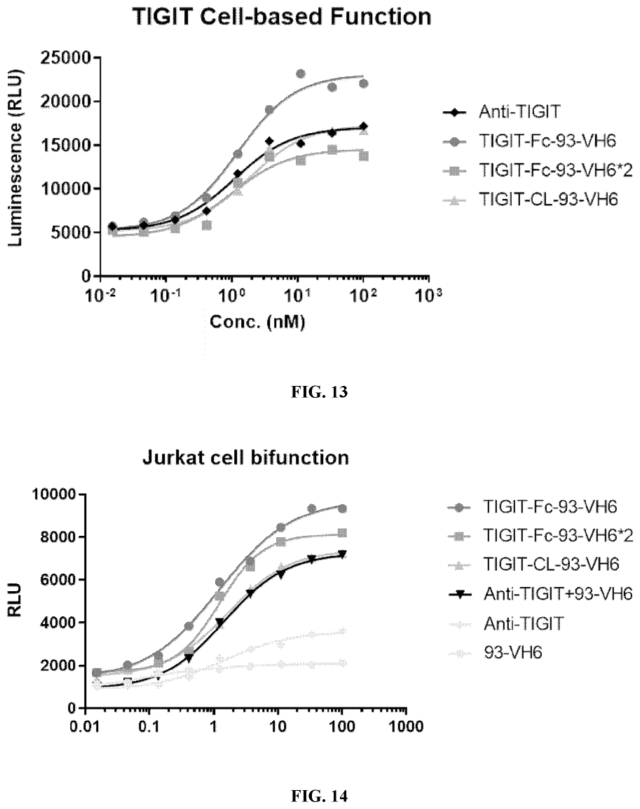

With this assay, the TIGIT-Fc-93-VH6 bsAb molecule showed superior efficacy in blocking TIGIT/CD155 signaling to enhance Jurkat cell activation when compared with their parental TIGIT antibody (FIG. 13). The other two formats, TIGIT-Fc-93-VH6*2 and TIGIT-CL-93-VH6 bsAbs showed comparable TIGIT blocking activity as the parental TIGIT antibody.

### Example 19. Synergistic Effect of PD-L1/TIGIT bsAbs In Vitro

To evaluate the synergistic effect of PD-L1/TIGIT bsAbs in boosting T cells activation, we established a robust in vitro cell-based bifunctional assay. In brief, human TIGIT, CD226 and PD1 were simultaneously overexpressed on Jurkat T cells, while their individual ligands CD155 and PD-L1 were overexpressed on Raji cells. When these two cell types were cocultured in the presence of super antigen, the negative signaling delivered on Jurkat cells by both TIGIT-CD155 and PD-1-PD-L1 interaction synergistically inhibited Jurkat cell activation, which is indicated by luciferase reporter gene expression.

As shown in FIG. 14, when serially diluted TIGIT antibody or PD-L1 antibody were added to the culture systems, antibodies could dose-dependently enhance luciferase expression of Jurkat-TIGIT-CD226-PD-1 cells. However, combination of anti-TIGIT and anti-PD-L1 antibodies significantly enhanced luciferase production, showing a strong synergistic effect of these two antibodies. Of note, PD-L1/TIGIT bsAb Formats TIGIT-Fc-93-VH6 and TIGIT-Fc-PD-93-VH6*2 showed even significantly enhanced T cell activation than the combo treatment, whereas TIGIT-CL-93-VH6 bsAb showed comparable T cell activation as the combo treatment.

To further confirm the observation in Jurkat cell line-based bifunctional assay, the synergistic effect of PD-L1/TIGIT bsAbs on human PBMC derived primary CD8+ T cells activation was further studied. In brief, CHO-K1 cells constitutively expressing an engineered T cell receptor (TCR) activator, human CD155 and PD-L1 (CHO-TCR-CD155-PD-L1 cells) were seeded at a density of 35,000 cells per well and incubated overnight. Purified CD8+ T cells isolated from two healthy donors were incubated with CHO-TCR-CD155-PD-L1 cells at a density of 50,000 cells per well. Serially diluted PD-L1/TIGIT bsAbs, anti-TIGIT, anti-PD-L1 or the combination of these two antibodies were then added to the co-culture system for 3 days and the culture medium was collected for IFN-γ measurement using a standard ELISA kit.

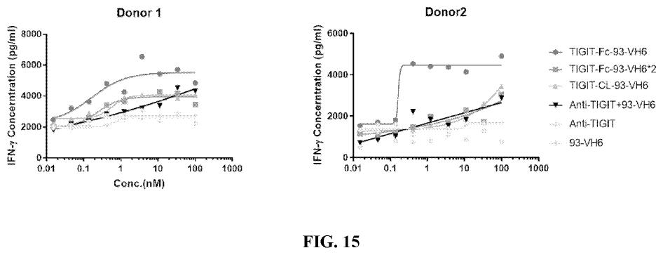

As shown in FIG. 15, while anti-TIGIT or anti-PD-L1 antibodies could barely stimulate IFN-γ production by primary CD8+ T cells, the combination of these two antibodies significantly enhanced IFN-γ production in a concentration-dependent manner. Most importantly, TIGIT-Fc-93-VH6 bsAb showed significantly superior efficacy than the combo treatment in T cell activation induced IFN-γ production, demonstrating a strong synergistic effect of this PD-L1/TIGIT bsAb format on primary CD8+ T cell activation in vitro.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A single domain antibody or a polypeptide comprising the single domain antibody, wherein the single domain antibody has binding specificity to the human PD-L1 protein and comprises a complementarity determining region 1 (CDR1), a CDR2 and a CDR3 of anyone selected from the group consisting of SEQ ID NO: 1-36, 114-122 and 123-130.

2. The antibody or polypeptide of claim 1, wherein the single domain antibody has binding specificity to the human PD-L1 protein and comprises a complementarity determining region 1 (CDR1), a CDR2 and a CDR3 of anyone selected from the group consisting of SEQ ID NO: 1-36, 114-122 and 123-130, and the CDR1, CDR2, and CDR3 are according to Kabat numbering scheme.

3. (canceled)

4. The antibody or polypeptide of claim 1, wherein the CDR1 comprises the amino acid sequence of SEQ ID NO:55, the CDR2 comprises the amino acid sequence of SEQ ID NO:56, and the CDR3 comprises the amino acid sequence of SEQ ID NO:57.

5. The antibody or polypeptide of claim 4, wherein the antibody is humanized.

6. The antibody or polypeptide of claim 5, wherein the humanized antibody comprises one or more back mutations selected from the group consisting of 37F, 47F, 49A, 78V and 94A, according to Kabat numbering.

7. The antibody or polypeptide of claim 5, wherein the humanized antibody comprises back mutations of 37F, 47F, 49A, 78V and 94A, according to Kabat numbering.

8. The antibody or polypeptide of claim 5, wherein the antibody comprises an amino acid sequence of anyone selected from the group consisting of SEQ ID NO: 114-122.

9. The antibody or polypeptide of claim 8, wherein the antibody comprises the amino acid sequence of SEQ ID NO: 119.

10. The antibody or polypeptide of claim 1, wherein the CDR1 comprises the amino acid sequence of SEQ ID NO:113, the CDR2 comprises the amino acid sequence of SEQ ID NO:49, and the CDR3 comprises the amino acid sequence of SEQ ID NO:50.

11. The antibody or polypeptide of claim 10, wherein the antibody is humanized.

12. The antibody or polypeptide of claim 11, wherein the humanized antibody comprises one or more back mutations selected from the group consisting of 37Y, 44Q, 45R, 49A, 68A, 93R, and 94V, according to Kabat numbering.

13. The antibody or polypeptide of claim 11, wherein the humanized antibody comprises back mutations of 37Y, 44Q, 45R, 49A, 68A, 93R, and 94V, according to Kabat numbering.

14. The antibody or polypeptide of claim 11, wherein the antibody comprises an amino acid sequence of anyone selected from the group consisting of SEQ ID NO: 123-130.

15. The antibody or polypeptide of claim 14, wherein the antibody comprises the amino acid sequence of SEQ ID NO: 127 or 130.

16. The antibody or polypeptide of claim 1, wherein the polypeptide is a bispecific antibody having a binding specificity to an antigen different from PD-L1.

17. A bispecific antibody comprising the antibody of claim 1 and a second antibody or antigen-binding fragment having binding specificity to a target antigen that is not PD-L1.

18. A polynucleotide encoding the antibody or polypeptide of claim 1.

19. A vector comprising the polynucleotide of claim 18.

20. A cell comprising the vector of claim 19.

21. A composition comprising
(1) the antibody or polypeptide of claim 1, and
(2) a pharmaceutically acceptable carrier.

22. A method of treating cancer in a patient in need thereof, comprising administering to the patient an effective amount of the antibody or polypeptide of claim 1.

23. (canceled)

24. The method of claim 22, wherein the cancer is a solid tumor.

25. The method of claim 22, wherein the cancer is selected from the group consisting of bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, oesophageal cancer, ovarian cancer, renal cancer, melanoma, prostate cancer and thyroid cancer.


SEQUENCE LISTING (184 sequences)

SEQ ID NO: 1 - protein, 126 aa, Artificial Sequence
  Synthetic
EVQLVESGGGLVQAGDSLTLSCAASGRTFSSYAMGWFRQAPGKEREFVARITWTGRSTSYADSVKGRFTISRDNAKNRVYLRMNSLKPEDTAVYYCAADLEGAMVSRRREIEYGHWGQGTQVTVSS

SEQ ID NO: 2 - protein, 122 aa, Artificial Sequence
  Synthetic
EVDLVESGGGLVQAGGSLRLSCAASGGSTFAMAWLRQAPGKEREFVAAVGRSPRSPGITYYADSVKGRFTISRDNAKNTVYLQMNSLKPEDTAVYYCAAGGILGPRAHYDYWGQGTQVTVSS

SEQ ID NO: 3 - protein, 122 aa, Artificial Sequence
  Synthetic
QVQLVESGGGLVQAGGSLRLSCAASGRTFSRYAMGWFRQAPGKEREFVAAISWSGGTTNYADSVKGRFTISRDNAKITVYLQMNSLKPEDTAFYYCAAGKRLTLRSSGYKYWGQGTQVTVSS

SEQ ID NO: 4 - protein, 122 aa, Artificial Sequence
  Synthetic
AVQLVESGGGLVQAGGSLRLSCAASRGSTFAMAWIRQAPGKEREFVAAVGRSPRGPGITYYADSVKGRFTISRDNANNTVYLQMNSLKPEDTAVYYCAAGGILGPRAQYDYWGQGTQVTVSS

SEQ ID NO: 5 - protein, 122 aa, Artificial Sequence
  Synthetic
QVQLVESGGGLVQAGSSLRLSCAASRGSTFAMAWIRQAPGKEREFVAAVGRSPRGPGITYYADSVKGRFTISRDNANNTVYLQMNSLKPEDTAVYYCAAGGILGPRAQYDYWGQGTQVTVSS

SEQ ID NO: 6 - protein, 121 aa, Artificial Sequence
  Synthetic
QVQLVESGGGLVQPGGSLRLSCAASGSIFSSGTNFSDSKIDWYRQAPGKQRDWIAGIFSTGSTIYEDSVKGRFAISRDNAKNMGYLQMNSLKPEDTAVYYCRVIGRGILAWGQGTQVTVSS

SEQ ID NO: 7 - protein, 122 aa, Artificial Sequence
  Synthetic
QVQLVESGGGLVQAGGSLRLSCAASRGSTFAMAWIRQAPRKEREFVPAVGRSPLGPVITYYADSVKGRFTISRDNANNTVYLQMNSLKPEDTAVDYCASCGILGPRAHYDYWGQGTHVTVSS

SEQ ID NO: 8 - protein, 126 aa, Artificial Sequence
  Synthetic
EVQLVESGGGLVQAGDSLTLSCAASGRTFSSYAMGWFRQAPGKEREFVARITWSGRSTSYADSVKGRFTISRDNAKNRVYLRMNSLKPEDTAVYYCAADLEGAMVSRRREIEYGQWGQGTQVTVSS

SEQ ID NO: 9 - protein, 122 aa, Artificial Sequence
  Synthetic
QVQLVESGGGLVQAGGSLRLSCAASGGSTFAMAWIRQAPGKEREFVAAVGRSPRSPGITYYADSVKGRFTISRDNANNTVYLQMNSLKPEDTAVYYCAAGGILGPRAQYDYWGQGTQVTVSS

SEQ ID NO: 10 - protein, 122 aa, Artificial Sequence; identical to SEQ ID NO: 5
  Synthetic

SEQ ID NO: 11 - protein, 126 aa, Artificial Sequence
  Synthetic
QVQLVESVGGLVQPGDSLRLSCLASGRTFTFRHYVMGWFRQAPGKEREFVAAISWSGSGSYYADSVKGRFTISRDNSKNMVFLQMNGLKPEDTAVYYCAADMTTRMSQASREYDYWGQGTQVTVSS

SEQ ID NO: 12 - protein, 122 aa, Artificial Sequence
  Synthetic
EVQLVESGGGLVQAGGSLRLSCAASGGSTFAMAWLRQAPGKEREFVAAVGRSPRSPGITYYADSVKGRFTISRDNAKNTVWLQMNSLKPEDTAVYYCAAGGILGPRAEYDYWGQGTRVTVSS

SEQ ID NO: 13 - protein, 126 aa, Artificial Sequence
  Synthetic
QVHLVESGGGLVQAGDSLTLSCAASGRTFSSYAMGWFRQAPGKEREFVARITWSGRSTSYADSVKGRFTISRDNAKNRVYLRMNSLKPEDTAVYYCAADLEGAMVSRRREIEYGHWGQGTQVTVSS

SEQ ID NO: 14 - protein, 127 aa, Artificial Sequence
  Synthetic
QVQLVESGGELVQAGGSLRLSCAASGRTFSSYAMGWFRQGPGKEREFVAAISASGGRTYYADSVKGRFTISRDNAKNTVYLQMNSLKPEDTAVYYCAVAGPRIRIATITLSREYDYWGQGTQVTVSS

SEQ ID NO: 15 - protein, 115 aa, Artificial Sequence
  Synthetic
AVQLVESGGGLVQAGGSLRLSCVASEIAFSVFDMGWYRQAPGKQRELAASIGHDGRINYADSVKGRFTISRDNAKNTVHLQMNTLKSEDTAVYYCNARNSFRDLWGQGTQVTVSS

SEQ ID NO: 16 - protein, 127 aa, Artificial Sequence
  Synthetic
AVQLVESGGGLVQPGGSLRLSCAASGRSFSGYAMGWFRQAPGKEREFVSAISGSGRNTYYADSVKGRFTISRDNAKNTMYLQMNSLKPEDTAVYYCAVAGPAITIATMTLRGKYDYWGQGTQVTVSS

SEQ ID NO: 17 - protein, 127 aa, Artificial Sequence
  Synthetic
QVHLVESGGGLVQAGDSLRLSCAASGRTFSSRAMGWFRQAPGKEREFVAAISASGSRTYYADSVKGRFTISRDNAKNTVYLQMNSLKPEDTAVYYCAAAGPRITIATMTLSREYDYWGQGTQVTVSS

SEQ ID NO: 18 - protein, 127 aa, Artificial Sequence
  Synthetic
EVQLVESGGGLVQPGGSLRLSCAASGRTFSSYALGWFRQAPGKEREFVAAISASGLRTYYADSVKGRFTISRDNAKNTVYLQMNSLKPEDTAVYYCAEAGPRIRIATMTLSREYDYWGQGTLVTVSS

SEQ ID NO: 19 - protein, 127 aa, Artificial Sequence
  Synthetic
QVQLVESGGGLVQAGGSLRLSCAASGRTFSSYAMGWFRQAPGKEREFATAISASGRSTYYADSVKGRFTISRDNAKNTVYLQMNSLKPEDTAVYYCAQGGPSITIRTMGSSSKYDYWGRGTQVTVSS

SEQ ID NO: 20 - protein, 127 aa, Artificial Sequence
  Synthetic
QVHLVESGGGLVQAGGSLRLSCAASGRTFSSYAMGWFRQAPGKEREFVAAVSASGGRSYYVDSVKGRFTISRDNAKNTVYLQMNSLKPEDTAVYYCAAAGRSITIATMTERYKYDYWGQGTQVTVSS

SEQ ID NO: 21 - protein, 127 aa, Artificial Sequence
  Synthetic
EVQLVESGGGLVQAGGSLRLSCAASGRSFSGYAMGWFRQAPGKERDFVAAISGTGGSTYYVDSVKGRFTISRDNAKNTMYLQMNSLKPEDTAVYYCAVAGPAITIATMTLRGKYDYWGQGTQVTVSS

SEQ ID NO: 22 - protein, 118 aa, Artificial Sequence
  Synthetic
AVQLVESGGSLRLSCAASGRTFSSYAMGWFRQAPGKEREFVAAISGSGARTYYADSVKGRFTISRANTKNTVYLQMNSLKPEDTAVYYCAADATRIASVDVPKSWGYWGQGTQVTVSS

SEQ ID NO: 23 - protein, 126 aa, Artificial Sequence
  Synthetic
QLHFVESGGGLVQAGGSLRLACAASGRTFSGYARTWFRQAPGKEREFVAAISGSGASAYYADSVKGRFTISRDNAKNTVYLQMNSLKPEDTAVYYCAADQSIRIATMRTHAAYGYWGQGTQVTVSS

SEQ ID NO: 24 - protein, 122 aa, Artificial Sequence
  Synthetic
QVQLVESVGGLVQAGGSLSLSCAASGGSTFAMAWLRQAPGKEREFVAAVGRSPRGPGITNYADSVKGRFTISRDNAKNTVYLQMNSLKPEDTAVYYCAAGGILGPRAQYDYWGQGTRVTVSS

SEQ ID NO: 25 - protein, 125 aa, Artificial Sequence
  Synthetic
EVHLVESGGGLVQPGGSLRLSCAHSGSIRSINVMNWYRQVPGKQRELVATITAGGSTNYADSVKGRFTISRDNALNTAALQMNSLRPEDTAVYYCHADKILTYNGVIYRAEYDVWGQGTQVTVSS

SEQ ID NO: 26 - protein, 127 aa, Artificial Sequence
  Synthetic
QVQLVESGGGLVQAGGSLRLSCAASGRTFSGYAMGWFRQAPGKEREFVAAISGSGGRTYYVDSAKGRFTISRDNAKNTMYLQMNSLKPEDTAVYYCAVAGPAITIATMTLRGKYDYWGQGTQVTVSS

SEQ ID NO: 27 - protein, 125 aa, Artificial Sequence
  Synthetic
QVQLVESGGGLVQPGGSLRLSCAASESIRSINVMNWYRQAPGKQRELVATITSGGTTTYADSVKGRFTSSRDNAKNTVALQMNSLRPEDTAVYYCHADKVLYYNGVIYGAEYDVWGQGTQVTVSS

SEQ ID NO: 28 - protein, 125 aa, Artificial Sequence
  Synthetic
QLQLVESGGGLVQPGGSLRLSCAPSGSIRSINVMNWYRQAPGKQRDLVATITSGGSTNYADSVKGRFIISRDNAKNTVALQMNSLRPEDTAVYYCHADKVLYYNGVLYGAEYDVWGQGTQVTVSS

SEQ ID NO: 29 - protein, 131 aa, Artificial Sequence
  Synthetic
EVHLVESGGGLVQAGGSLRLSCEVSGSIFSGTHFSFNTMGWYRQAPGKQRELVALGRGSRGINYADSVKGRFTFSSDNAKNTIFLQMNNLEPEDTGNYTCYVRRPSGSYAGQYYPDSSEYWGQGTQVTVSS

SEQ ID NO: 30 - protein, 111 aa, Artificial Sequence
  Synthetic
QPQVVESGGGLVQAGGSLRLSCVASGSNFAFEYIAWYRQAPGKEREVVALISPQSITTYADSVKGRFTISRDNAKSTVYLQMNSLKPEDTAVYYCHDREYWGQGTQVTVSS

SEQ ID NO: 31 - protein, 125 aa, Artificial Sequence
  Synthetic
QLHFVESGGGLVQPGGSLRLSCAASGSIRSINVMNYYRQAPGKQRELVATISSVGSTNYADSVKGRFTISRDNAQNTVALQMNSLRPEDTAVYYCHADKVLYYNGVMYGVESDVWGQGTQVTVSS

SEQ ID NO: 32 - protein, 128 aa, Artificial Sequence
  Synthetic
QVQLVESGGGEVQPGGSLRLSCAASGPTFSRYIMGWFRQAPGKEREFVAAISRIGGITYYTDSVKGRFTISRDNAKNTVYLQMNSLEPEDTASYYCAAKSSSSSSKYTARGADAYDYWGQGTQVTVSS

SEQ ID NO: 33 - protein, 117 aa, Artificial Sequence
  Synthetic
AVQLVESGGGLVQPGGSLRLSCAASRSVFSVLVMGWYRQAPGQQRELVATISNEGYSNYADSVKGRFAISRDNAKKTVYLQMNSLKPEDTAVYYCNAAWGNGRYTYWGQGTQVTVSS

SEQ ID NO: 34 - protein, 125 aa, Artificial Sequence
  Synthetic
EVHLVESGGGLVQPGGSLRLSCAHSGSIRSINVMNWYRQVPGKQRELVATITAGGSTNYADSVKGRFTISRDNAKNSAALQMNSLRPEDTAVYYCHADKVLSYNGVIYRAEYDVWGQGTQVTVSS

SEQ ID NO: 35 - protein, 126 aa, Artificial Sequence
  Synthetic
ELQLVESGGGLVQAGGSLRLSCAVSESIFKFPRMGWYRQGPGDQRDLVALSRSSGSTEYADFAKGRFTISRDNSKNTVYLQMNSLKPEDSGTYYCYVRRPSGSFNGRWYTDPSDDWGQGTRVTVSS

SEQ ID NO: 36 - protein, 113 aa, Artificial Sequence
  Synthetic
QVQLVESGGGLVQPGGSLRLSCAASARSINGMEWYRQAPGERRELVAGITAGGSAYYTDTVKGRFTISRDNAENTGYLQMNSLSPDDTAVYYCRRQYGPNWYWGQGTQVTVSS

SEQ ID NO: 37 - protein, 5 aa, Artificial Sequence
  Synthetic
SYAMG

SEQ ID NO: 38 - protein, 17 aa, Artificial Sequence
  Synthetic
RITWTGRSTSYADSVKG

SEQ ID NO: 39 - protein, 17 aa, Artificial Sequence
  Synthetic
DLEGAMVSRRREIEYGH

SEQ ID NO: 40 - protein, 4 aa, Artificial Sequence
  Synthetic
  MISC_FEATURE (4)..(4): Xaa is any amino acid or empty
AMAX

SEQ ID NO: 41 - protein, 20 aa, Artificial Sequence
  Synthetic
AVGRSPRSPGITYYADSVKG

SEQ ID NO: 42 - protein, 12 aa, Artificial Sequence
  Synthetic
GGILGPRAHYDY

SEQ ID NO: 43 - protein, 5 aa, Artificial Sequence
  Synthetic
RYAMG

SEQ ID NO: 44 - protein, 17 aa, Artificial Sequence
  Synthetic
AISWSGGTTNYADSVKG

SEQ ID NO: 45 - protein, 13 aa, Artificial Sequence
  Synthetic
GKRLTLRSSGYKY

SEQ ID NO: 46 - protein, 20 aa, Artificial Sequence
  Synthetic
AVGRSPRGPGITYYADSVKG

SEQ ID NO: 47 - protein, 12 aa, Artificial Sequence
  Synthetic
GGILGPRAQYDY

SEQ ID NO: 48 - protein, 11 aa, Artificial Sequence
  Synthetic
SGTNFSDSKID

SEQ ID NO: 49 - protein, 16 aa, Artificial Sequence
  Synthetic
GIFSTGSTIYEDSVKG

SEQ ID NO: 50 - protein, 7 aa, Artificial Sequence
  Synthetic
IGRGILA

SEQ ID NO: 51 - protein, 20 aa, Artificial Sequence
  Synthetic
AVGRSPLGPVITYYADSVKG

SEQ ID NO: 52 - protein, 12 aa, Artificial Sequence
  Synthetic
CGILGPRAHYDY

SEQ ID NO: 53 - protein, 17 aa, Artificial Sequence
  Synthetic
RITWSGRSTSYADSVKG

SEQ ID NO: 54 - protein, 17 aa, Artificial Sequence
  Synthetic
DLEGAMVSRRREIEYGQ

SEQ ID NO: 55 - protein, 6 aa, Artificial Sequence
  Synthetic
RHYVMG

SEQ ID NO: 56 - protein, 17 aa, Artificial Sequence
  Synthetic
AISWSGSGSYYADSVKG

SEQ ID NO: 57 - protein, 15 aa, Artificial Sequence
  Synthetic
DMTTRMSQASREYDY

SEQ ID NO: 58 - protein, 12 aa, Artificial Sequence
  Synthetic
GGILGPRAEYDY

SEQ ID NO: 59 - protein, 17 aa, Artificial Sequence
  Synthetic
AISASGGRTYYADSVKG

SEQ ID NO: 60 - protein, 18 aa, Artificial Sequence
  Synthetic
AGPRIRIATITLSREYDY

SEQ ID NO: 61 - protein, 5 aa, Artificial Sequence
  Synthetic
VFDMG

SEQ ID NO: 62 - protein, 16 aa, Artificial Sequence
  Synthetic
SIGHDGRINYADSVKG

SEQ ID NO: 63 - protein, 7 aa, Artificial Sequence
  Synthetic
RNSFRDL

SEQ ID NO: 64 - protein, 5 aa, Artificial Sequence
  Synthetic
GYAMG

SEQ ID NO: 65 - protein, 17 aa, Artificial Sequence
  Synthetic
AISGSGRNTYYADSVKG

SEQ ID NO: 66 - protein, 18 aa, Artificial Sequence
  Synthetic
AGPAITIATMTLRGKYDY

SEQ ID NO: 67 - protein, 5 aa, Artificial Sequence
  Synthetic
SRAMG

SEQ ID NO: 68 - protein, 17 aa, Artificial Sequence
  Synthetic
AISASGSRTYYADSVKG

SEQ ID NO: 69 - protein, 18 aa, Artificial Sequence
  Synthetic
AGPRITIATMTLSREYDY

SEQ ID NO: 70 - protein, 5 aa, Artificial Sequence
  Synthetic
SYALG

SEQ ID NO: 71 - protein, 17 aa, Artificial Sequence
  Synthetic
AISASGLRTYYADSVKG

SEQ ID NO: 72 - protein, 18 aa, Artificial Sequence
  Synthetic
AGPRIRIATMTLSREYDY

SEQ ID NO: 73 - protein, 17 aa, Artificial Sequence
  Synthetic
AISASGRSTYYADSVKG

SEQ ID NO: 74 - protein, 18 aa, Artificial Sequence
  Synthetic
GGPSITIRTMGSSSKYDY

SEQ ID NO: 75 - protein, 17 aa, Artificial Sequence
  Synthetic
AVSASGGRSYYVDSVKG

SEQ ID NO: 76 - protein, 18 aa, Artificial Sequence
  Synthetic
AGRSITIATMTERYKYDY

SEQ ID NO: 77 - protein, 17 aa, Artificial Sequence
  Synthetic
AISGTGGSTYYVDSVKG

SEQ ID NO: 78 - protein, 17 aa, Artificial Sequence
  Synthetic
AISGSGARTYYADSVKG

SEQ ID NO: 79 - protein, 16 aa, Artificial Sequence
  Synthetic
DATRIASVDVPKSWGY

SEQ ID NO: 80 - protein, 5 aa, Artificial Sequence
  Synthetic
GYART

SEQ ID NO: 81 - protein, 17 aa, Artificial Sequence
  Synthetic
AISGSGASAYYADSVKG

SEQ ID NO: 82 - protein, 17 aa, Artificial Sequence
  Synthetic
DQSIRIATMRTHAAYGY

SEQ ID NO: 83 - protein, 20 aa, Artificial Sequence
  Synthetic
AVGRSPRGPGITNYADSVKG

SEQ ID NO: 84 - protein, 5 aa, Artificial Sequence
  Synthetic
INVMN

SEQ ID NO: 85 - protein, 16 aa, Artificial Sequence
  Synthetic
TITAGGSTNYADSVKG

SEQ ID NO: 86 - protein, 17 aa, Artificial Sequence
  Synthetic
DKILTYNGVIYRAEYDV

SEQ ID NO: 87 - protein, 17 aa, Artificial Sequence
  Synthetic
AISGSGGRTYYVDSAKG

SEQ ID NO: 88 - protein, 16 aa, Artificial Sequence
  Synthetic
TITSGGTTTYADSVKG

SEQ ID NO: 89 - protein, 17 aa, Artificial Sequence
  Synthetic
DKVLYYNGVIYGAEYDV

SEQ ID NO: 90 - protein, 16 aa, Artificial Sequence
  Synthetic
TITSGGSTNYADSVKG

SEQ ID NO: 91 - protein, 17 aa, Artificial Sequence
  Synthetic
DKVLYYNGVLYGAEYDV

SEQ ID NO: 92 - protein, 10 aa, Artificial Sequence
  Synthetic
GTHFSFNTMG

SEQ ID NO: 93 - protein, 16 aa, Artificial Sequence
  Synthetic
LGRGSRGINYADSVKG

SEQ ID NO: 94 - protein, 18 aa, Artificial Sequence
  Synthetic
RRPSGSYAGQYYPDSSEY

SEQ ID NO: 95 - protein, 5 aa, Artificial Sequence
  Synthetic
FEYIA

SEQ ID NO: 96 - protein, 16 aa, Artificial Sequence
  Synthetic
LISPQSITTYADSVKG

SEQ ID NO: 97 - protein, 4 aa, Artificial Sequence
  Synthetic
  MISC_FEATURE (4)..(4): Xaa is any amino acid or empty
REYX

SEQ ID NO: 98 - protein, 16 aa, Artificial Sequence
  Synthetic
TISSVGSTNYADSVKG

SEQ ID NO: 99 - protein, 17 aa, Artificial Sequence
  Synthetic
DKVLYYNGVMYGVESDV

SEQ ID NO: 100 - protein, 5 aa, Artificial Sequence
  Synthetic
RYIMG

SEQ ID NO: 101 - protein, 17 aa, Artificial Sequence
  Synthetic
AISRIGGITYYTDSVKG

SEQ ID NO: 102 - protein, 19 aa, Artificial Sequence
  Synthetic
KSSSSSSKYTARGADAYDY

SEQ ID NO: 103 - protein, 5 aa, Artificial Sequence
  Synthetic
VLVMG

SEQ ID NO: 104 - protein, 16 aa, Artificial Sequence
  Synthetic
TISNEGYSNYADSVKG

SEQ ID NO: 105 - protein, 9 aa, Artificial Sequence
  Synthetic
AWGNGRYTY

SEQ ID NO: 106 - protein, 17 aa, Artificial Sequence
  Synthetic
DKVLSYNGVIYRAEYDV

SEQ ID NO: 107 - protein, 5 aa, Artificial Sequence
  Synthetic
FPRMG

SEQ ID NO: 108 - protein, 16 aa, Artificial Sequence
  Synthetic
LSRSSGSTEYADFAKG

SEQ ID NO: 109 - protein, 18 aa, Artificial Sequence
  Synthetic
RRPSGSFNGRWYTDPSDD

SEQ ID NO: 110 - protein, 4 aa, Artificial Sequence
  Synthetic
  MISC_FEATURE (4)..(4): Xaa is any amino acid or empty
GMEX

SEQ ID NO: 111 - protein, 16 aa, Artificial Sequence
  Synthetic
GITAGGSAYYTDTVKG

SEQ ID NO: 112 - protein, 7 aa, Artificial Sequence
  Synthetic
QYGPNWY

SEQ ID NO: 113 - protein, 11 aa, Artificial Sequence
  Synthetic
SGTQFSDSKID

SEQ ID NO: 114 - protein, 126 aa, Artificial Sequence
  Synthetic
EVQLVESGGGLVQPGGSLRLSCAASGFTFTFRHYVMGWVRQAPGKEREWVSAISWSGSGSYYADSVKGRFTISRDNSKNTLYLQMNSLRAEDTAVYYCAKDMTTRMSQASREYDYWGQGTLVTVSS

SEQ ID NO: 115 - protein, 126 aa, Artificial Sequence
  Synthetic
EVQLVESGGGLVQPGGSLRLSCAASGFTFTFRHYVMGWVRQAPGKEREWVSAISWSGSGSYYADSVKGRFTISRDNSKNTVYLQMNSLRAEDTAVYYCAADMTTRMSQASREYDYWGQGTLVTVSS

SEQ ID NO: 116 - protein, 126 aa, Artificial Sequence
  Synthetic
EVQLVESGGGLVQPGGSLRLSCAASGFTFTFRHYVMGWFRQAPGKGLEFVAAISWSGSGSYYADSVKGRFTISRDNSKNTLYLQMNSLRAEDTAVYYCAKDMTTRMSQASREYDYWGQGTLVTVSS

SEQ ID NO: 117 - protein, 126 aa, Artificial Sequence
  Synthetic
EVQLVESGGGLVQPGGSLRLSCAASGFTFTFRHYVMGWFRQAPGKGLEFVAAISWSGSGSYYADSVKGRFTISRDNSKNTVYLQMNSLRAEDTAVYYCAADMTTRMSQASREYDYWGQGTLVTVSS

SEQ ID NO: 118 - protein, 126 aa, Artificial Sequence
  Synthetic
EVQLVESGGGLVQPGGSLRLSCAASGFTFTFRHYVMGWFRQAPGKEREFVAAISWSGSGSYYADSVKGRFTISRDNSKNTLYLQMNSLRAEDTAVYYCAKDMTTRMSQASREYDYWGQGTLVTVSS

SEQ ID NO: 119 - protein, 126 aa, Artificial Sequence
  Synthetic
EVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQASREYDYWGQGTLVTVSS

SEQ ID NO: 120 - protein, 126 aa, Artificial Sequence
  Synthetic
EVQLVESGGGLVQPGGSLRLSCAASGFTFTFRHYVMGWFRQAPGKGLEFVAAISWSGSGSYYADSVKGRFTISRDNSKNTLYLQMNSLRAEDTAVYYCAADMTTRMSQASREYDYWGQGTLVTVSS

SEQ ID NO: 121 - protein, 126 aa, Artificial Sequence
  Synthetic
EVQLVESGGGLVQPGGSLRLSCAASGRTFTFRHYVMGWFRQAPGKGLEFVAAISWSGSGSYYADSVKGRFTISRDNSKNTVYLQMNSLRAEDTAVYYCAADMTTRMSQASREYDYWGQGTLVTVSS

SEQ ID NO: 122 - protein, 126 aa, Artificial Sequence
  Synthetic
EVQLVESGGGLVQPGGSLRLSCAASGRTFTFRHYVMGWFRQAPGKGLEFVAAISWSGSGSYYADSVKGRFTISRDNSKNTLYLQMNSLRAEDTAVYYCAADMTTRMSQASREYDYWGQGTLVTVSS

SEQ ID NO: 123 - protein, 121 aa, Artificial Sequence
  Synthetic
EVQLVESGGGLVQPGGSLRLSCAASGSIFSSGTQFSDSKIDWVRQAPGKGLEWVSGIFSTGSTIYEDSVKGRFTISRDNAKNSLYLQMNSLRAEDTAVYYCARIGRGILAWGQGTLVTVSS

SEQ ID NO: 124 - protein, 121 aa, Artificial Sequence
  Synthetic
EVQLVESGGGLVQPGGSLRLSCAASGSIFSSGTQFSDSKIDWYRQAPGKQREWVSGIFSTGSTIYEDSVKGRFTISRDNAKNSLYLQMNSLRAEDTAVYYCARIGRGILAWGQGTLVTVSS

SEQ ID NO: 125 - protein, 121 aa, Artificial Sequence
  Synthetic
EVQLVESGGGLVQPGGSLRLSCAASGSIFSSGTQFSDSKIDWYRQAPGKQREWVSGIFSTGSTIYEDSVKGRFTISRDNAKNSLYLQMNSLRAEDTAVYYCRVIGRGILAWGQGTLVTVSS

SEQ ID NO: 126 - protein, 121 aa, Artificial Sequence
  Synthetic
EVQLVESGGGLVQPGGSLRLSCAASGSIFSSGTQFSDSKIDWYRQAPGKQREWVAGIFSTGSTIYEDSVKGRFTISRDNAKNSLYLQMNSLRAEDTAVYYCRVIGRGILAWGQGTLVTVSS

SEQ ID NO: 127 - protein, 121 aa, Artificial Sequence
  Synthetic
EVQLVESGGGLVQPGGSLRLSCAASGSIFSSGTQFSDSKIDWYRQAPGKQRDWVAGIFSTGSTIYEDSVKGRFAISRDNAKNSLYLQMNSLRAEDTAVYYCRVIGRGILAWGQGTLVTVSS

SEQ ID NO: 128 - protein, 121 aa, Artificial Sequence
  Synthetic
EVQLVESGGGLVQPGGSLRLSCAASGSIFSSGTQFSDSKIDWYRQAPGKQREWVAGIFSTGSTIYEDSVKGRFTISRDNAKNMLYLQMNSLRAEDTAVYYCRVIGRGILAWGQGTLVTVSS

SEQ ID NO: 129 - protein, 121 aa, Artificial Sequence
  Synthetic
EVQLVESGGGLVQPGGSLRLSCAASGSIFSSGTQFSDSKIDWYRQAPGKQREWIAGIFSTGSTIYEDSVKGRFAISRDNAKNMLYLQMNSLRAEDTAVYYCRVIGRGILAWGQGTLVTVSS

SEQ ID NO: 130 - protein, 121 aa, Artificial Sequence
  Synthetic
EVQLVESGGGLVQPGGSLRLSCAASGFTFSSGTQFSDSKIDWYRQAPGKGLVWVAGIFSTGSTIYEDSVKGRFTISRDNAKNTGYLQMNSLRAEDTAVYYCRVIGRGILAWGQGTLVTVSS

SEQ ID NO: 131 - protein, 118 aa, Artificial Sequence
  Synthetic
EVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSS

SEQ ID NO: 132 - protein, 113 aa, Artificial Sequence
  Synthetic
DIVMTQSPDSLAVSLGERATINCKSSQSVLYAGNNRNYLAWYQQKPGQPPKLLINQASTRASGVPDRFSGSGSGTEFTLIISSLQAEDVAIYYCQQYYTPPLAFGGGTKLEIK

SEQ ID NO: 133 - protein, 120 aa, Artificial Sequence
  Synthetic
QVQLVQSGAEVKKPGSSVKVSCKASGYTFSSYYMHWVRQAPGQGLEWMGEINPNNARINFNEKFKTRVTLTVDKSTSTAYMELSSLRSEDTAVYYCTRGYYRYGAWFGYWGQGTLVTVSS

SEQ ID NO: 134 - protein, 107 aa, Artificial Sequence
  Synthetic
DIQMTQSPSSLSASVGDRVTITCRASQDISDYLNWYQQKPGKAPKLLIYYISRLHSGVPSRFSGSGSGTDYTLTISSLQPEDFATYYCQQGHTLPWTFGGGTKVEIK

SEQ ID NO: 135 - protein, 5 aa, Artificial Sequence
  Synthetic
RAWMN

SEQ ID NO: 136 - protein, 19 aa, Artificial Sequence
  Synthetic
RIKRKTDGETTDYAAPVKG

SEQ ID NO: 137 - protein, 7 aa, Artificial Sequence
  Synthetic
SNRAFDI

SEQ ID NO: 138 - protein, 17 aa, Artificial Sequence
  Synthetic
KSSQSVLYAGNNRNYLA

SEQ ID NO: 139 - protein, 7 aa, Artificial Sequence
  Synthetic
QASTRAS

SEQ ID NO: 140 - protein, 9 aa, Artificial Sequence
  Synthetic
QQYYTPPLA

SEQ ID NO: 141 - protein, 5 aa, Artificial Sequence
  Synthetic
SYYMH

SEQ ID NO: 142 - protein, 17 aa, Artificial Sequence
  Synthetic
EINPNNARINFNEKFKT

SEQ ID NO: 143 - protein, 11 aa, Artificial Sequence
  Synthetic
GYYRYGAWFGY

SEQ ID NO: 144 - protein, 11 aa, Artificial Sequence
  Synthetic
RASQDISDYLN

SEQ ID NO: 145 - protein, 7 aa, Artificial Sequence
  Synthetic
YISRLHS

SEQ ID NO: 146 - protein, 9 aa, Artificial Sequence
  Synthetic
QQGHTLPWT

SEQ ID NO: 147 - protein, 594 aa, Artificial Sequence
  Synthetic
EVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQASREYDYWGQGTLVTVSSGGGGSGGGGSGGGGSGGGGSEVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 220 aa, Artificial Sequence
  Synthetic
DIVMTQSPDSLAVSLGERATINCKSSQSVLYAGNNRNYLAWYQQKPGQPPKLLINQASTRASGVPDRFSGSGSGTEFTLIISSLQAEDVAIYYCQQYYTPPLAFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 596 aa, Artificial Sequence
  Synthetic
EVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQASREYDYWGQGTLVTVSSGGGGSGGGGSGGGGSGGGGSQVQLVQSGAEVKKPGSSVKVSCKASGYTFSSYYMHWVRQAPGQGLEWMGEINPNNARINFNEKFKTRVTLTVDKSTSTAYMELSSLRSEDTAVYYCTRGYYRYGAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 214 aa, Artificial Sequence
  Synthetic
DIQMTQSPSSLSASVGDRVTITCRASQDISDYLNWYQQKPGKAPKLLIYYISRLHSGVPSRFSGSGSGTDYTLTISSLQPEDFATYYCQQGHTLPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 448 aa, Artificial Sequence
  Synthetic
EVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 366 aa, Artificial Sequence
  Synthetic
EVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQASREYDYWGQGTLVTVSSGGGGSGGGGSGGGGSGGGGSDIVMTQSPDSLAVSLGERATINCKSSQSVLYAGNNRNYLAWYQQKPGQPPKLLINQASTRASGVPDRFSGSGSGTEFTLIISSLQAEDVAIYYCQQYYTPPLAFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 153 - protein, 450 aa, Artificial Sequence
  Synthetic
QVQLVQSGAEVKKPGSSVKVSCKASGYTFSSYYMHWVRQAPGQGLEWMGEINPNNARINFNEKFKTRVTLTVDKSTSTAYMELSSLRSEDTAVYYCTRGYYRYGAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 360 aa, Artificial Sequence
  Synthetic
EVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQASREYDYWGQGTLVTVSSGGGGSGGGGSGGGGSGGGGSDIQMTQSPSSLSASVGDRVTITCRASQDISDYLNWYQQKPGKAPKLLIYYISRLHSGVPSRFSGSGSGTDYTLTISSLQPEDFATYYCQQGHTLPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 448 aa, Artificial Sequence; identical to SEQ ID NO: 151
  Synthetic

SEQ ID NO: 156 - protein, 366 aa, Artificial Sequence
  Synthetic
DIVMTQSPDSLAVSLGERATINCKSSQSVLYAGNNRNYLAWYQQKPGQPPKLLINQASTRASGVPDRFSGSGSGTEFTLIISSLQAEDVAIYYCQQYYTPPLAFGGGTKLEIKGGGGSGGGGSGGGGSGGGGSEVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQASREYDYWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 450 aa, Artificial Sequence; identical to SEQ ID NO: 153
  Synthetic

SEQ ID NO: 158 - protein, 360 aa, Artificial Sequence
  Synthetic
DIQMTQSPSSLSASVGDRVTITCRASQDISDYLNWYQQKPGKAPKLLIYYISRLHSGVPSRFSGSGSGTDYTLTISSLQPEDFATYYCQQGHTLPWTFGGGTKVEIKGGGGSGGGGSGGGGSGGGGSEVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQASREYDYWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - protein, 594 aa, Artificial Sequence
  Synthetic
EVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQASREYDYWGQGTLVTVSS

SEQ ID NO: 160 - protein, 220 aa, Artificial Sequence; identical to SEQ ID NO: 148
  Synthetic

SEQ ID NO: 161 - protein, 596 aa, Artificial Sequence
  Synthetic
QVQLVQSGAEVKKPGSSVKVSCKASGYTFSSYYMHWVRQAPGQGLEWMGEINPNNARINFNEKFKTRVTLTVDKSTSTAYMELSSLRSEDTAVYYCTRGYYRYGAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQASREYDYWGQGTLVTVSS

SEQ ID NO: 162 - protein, 214 aa, Artificial Sequence; identical to SEQ ID NO: 150
  Synthetic

SEQ ID NO: 163 - protein, 499 aa, Artificial Sequence
  Synthetic
EVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQASREYDYWGQGTLVTVSSGGGGSGGGGSGGGGSGGGGSEVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQASREYDYWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 450 aa, Artificial Sequence
  Synthetic
QVQLVQSGAEVKKPGSSVKVSCKASGYTFSSYYMHWVRQAPGQGLEWMGEINPNNARINFNEKFKTRVTLTVDKSTSTAYMELSSLRSEDTAVYYCTRGYYRYGAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 165 - protein, 214 aa, Artificial Sequence; identical to SEQ ID NO: 150
  Synthetic

SEQ ID NO: 166 - protein, 499 aa, Artificial Sequence
  Synthetic
EVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQASREYDYWGQGTLVTVSSGGGGSGGGGSGGGGSGGGGSEVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQASREYDYWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 167 - protein, 450 aa, Artificial Sequence
  Synthetic
QVQLVQSGAEVKKPGSSVKVSCKASGYTFSSYYMHWVRQAPGQGLEWMGEINPNNARINFNEKFKTRVTLTVDKSTSTAYMELSSLRSEDTAVYYCTRGYYRYGAWFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 214 aa, Artificial Sequence; identical to SEQ ID NO: 150
  Synthetic

SEQ ID NO: 169 - protein, 499 aa, Artificial Sequence; identical to SEQ ID NO: 163
  Synthetic

SEQ ID NO: 170 - protein, 469 aa, Artificial Sequence
  Synthetic
QVQLVQSGAEVKKPGSSVKVSCKASGYTFSSYYMHWVRQAPGQGLEWMGEINPNNARINFNEKFKTRVTLTVDKSTSTAYMELSSLRSEDTAVYYCTRGYYRYGAWFGYWGQGTLVTVSSGGGGSGGGGSGGGGSDIQMTQSPSSLSASVGDRVTITCRASQDISDYLNWYQQKPGKAPKLLIYYISRLHSGVPSRFSGSGSGTDYTLTISSLQPEDFATYYCQQGHTLPWTFGGGTKVEIKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 171 - protein, 119 aa, Artificial Sequence
  Synthetic
EVKLVESGGGLVQPGGSLRLSCAASGFTFSDYYMYWVRQAPGKRLEWVASITKGGGSTYYPDTLKGRFTISRDNAKNSLYLQMNRLRAEDTAVYYCARQSSYDFVMDYWGQGTTVTVSS

SEQ ID NO: 172 - protein, 107 aa, Artificial Sequence
  Synthetic
DIVMTQSPSSLSASVGDRVTITCKASQDVDTAVAWYQQKPGKAPKLLIYWASARHTGVPSRFSGSGSGTDFTFTISSLQPEDIATYYCQQYSNYPLTFGQGTKLEIK

SEQ ID NO: 173 - protein, 5 aa, Artificial Sequence
  Synthetic
DYYMY

SEQ ID NO: 174 - protein, 17 aa, Artificial Sequence
  Synthetic
SITKGGGSTYYPDTLKG

SEQ ID NO: 175 - protein, 10 aa, Artificial Sequence
  Synthetic
QSSYDFVMDY

SEQ ID NO: 176 - protein, 11 aa, Artificial Sequence
  Synthetic
KASQDVDTAVA

SEQ ID NO: 177 - protein, 7 aa, Artificial Sequence
  Synthetic
WASARHT

SEQ ID NO: 178 - protein, 9 aa, Artificial Sequence
  Synthetic
QQYSNYPLT

SEQ ID NO: 179 - protein, 595 aa, Artificial Sequence
  Synthetic
EVKLVESGGGLVQPGGSLRLSCAASGFTFSDYYMYWVRQAPGKRLEWVASITKGGGSTYYPDTLKGRFTISRDNAKNSLYLQMNRLRAEDTAVYYCARQSSYDFVMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQASREYDYWGQGTLVTVSS

SEQ ID NO: 180 - protein, 214 aa, Artificial Sequence
  Synthetic
DIVMTQSPSSLSASVGDRVTITCKASQDVDTAVAWYQQKPGKAPKLLIYWASARHTGVPSRFSGSGSGTDFTFTISSLQPEDIATYYCQQYSNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 181 - protein, 741 aa, Artificial Sequence
  Synthetic
EVKLVESGGGLVQPGGSLRLSCAASGFTFSDYYMYWVRQAPGKRLEWVASITKGGGSTYYPDTLKGRFTISRDNAKNSLYLQMNRLRAEDTAVYYCARQSSYDFVMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQASREYDYWGQGTLVTVSSGGGGSGGGGSGGGGSGGGGSEVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQASREYDYWGQGTLVTVSS

SEQ ID NO: 182 - protein, 449 aa, Artificial Sequence
  Synthetic
EVKLVESGGGLVQPGGSLRLSCAASGFTFSDYYMYWVRQAPGKRLEWVASITKGGGSTYYPDTLKGRFTISRDNAKNSLYLQMNRLRAEDTAVYYCARQSSYDFVMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 360 aa, Artificial Sequence
  Synthetic
DIVMTQSPSSLSASVGDRVTITCKASQDVDTAVAWYQQKPGKAPKLLIYWASARHTGVPSRFSGSGSGTDFTFTISSLQPEDIATYYCQQYSNYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEVQLVESGGGLVQPGGSLRLSCAASGFTFTFRHYVMGWFRQAPGKEREFVAAISWSGSGSYYADSVKGRFTISRDNSKNTVYLQMNSLRAEDTAVYYCAADMTTRMSQASREYDYWGQGTLVTVSS

SEQ ID NO: 184 - protein, 590 aa, Artificial Sequence
  Synthetic
EVKLVESGGGLVQPGGSLRLSCAASGFTFSDYYMYWVRQAPGKRLEWVASITKGGGSTYYPDTLKGRFTISRDNAKNSLYLQMNRLRAEDTAVYYCARQSSYDFVMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEVQLVESGGGLVQPGGSLRLSCAASGFTFSSGTQFSDSKIDWYRQAPGKGLVWVAGIFSTGSTIYEDSVKGRFTISRDNAKNTGYLQMNSLRAEDTAVYYCRVIGRGILAWGQGTLVTVSS